SEQUENCE LISTING (152 sequences)

SEQ ID NO: 1 - protein, 529 aa, Anopheles gambiae
DNDPLVVNTDKGRIRGITVDAPSGKKVDVWLGIPYAQPPVGPLRFRHPRPAEKWTGVLNTTTPPNSCVQIVDTVFGDFPGATMWNPNTPLSEDCLYINVVAPRPRPKNAAVMLWIFGGGFYSGTATLDVYDHRALASEENVIVVSLQYRVASLGFLFLGTPEAPGNAGLFDQNLALRWVRDNIHRFGGDPSRVTLFGESAGAVSVSLHLLSALSRDLFQRAILQSGSPTAPWALVSREEATLRALRLAEAVGCPHEPSKLSDAVECLRGKDPHVLVNNEWGTLGICEFPFVPVVDGAFLDETPQRSLASGRFKKTEILTGSNTEEGYYFIIYYLTELLRKEEGVTVTREEFLQAVRELNPYVNGAARQAIVFEYTDWTEPDNPNSNRDALDKMVGDYHFTCNVNEFAQRYAEEGNNVYMYLYTHRSKGNPWPRWTGVMHGDEINYVFGEPLNPTLGYTEDEKDFSRKIMRYWSNFAKTGNPNPNTASSEFPEWPKHTAHGRHYLELGLNTSFVGRGPRLRQCAFWKKYL

SEQ ID NO: 2 - protein, 527 aa, Mus musculus
EDPQLLVRVRGGQLRGIRLKAPGGPVSAFLGIPFAEPPVGSRRFMPPEPKRPWSGVLDATTFQNVCYQYVDTLYPGFEGTEMWNPNRELSEDCLYLNVWTPYPRPASPTPVLIWIYGGGFYSGAASLDVYDGRFLAQVEGAVLVSMNYRVGTFGFLALPGSREAPGNVGLLDQRLALQWVQENIAAFGGDPMSVTLFGESAGAASVGMHILSLPSRSLFHRAVLQSGTPNGPWATVSAGEARRRATLLARLVGCPNDTELIACLRTRPAQDLVDHEWHVLPQESIFRFSFVPVVDGDFLSDTPEALINTGDFQDLQVLVGVVKDEGSYFLVYGVPGFSKDNESLISRAQFLAGVRIGVPQASDLAAEAVVLHYTDWLHPEDPTHLRDAMSAVVGDHNVVCPVAQLAGRLAAQGARVYAYIFEHRASTLTWPLWMGVPHGYEIEFIFGLPLDPSLNYTTEERIFAQRLMKYWTNFARTGDPNDPRDSKSPQWPPYTTAAQQYVSLNLKPLEVRRGLRAQTCAFWNRFL

SEQ ID NO: 3 - protein, 527 aa, Mus musculus; identical to SEQ ID NO: 2

SEQ ID NO: 4 - protein, 532 aa, Electrophorus electricus
DPQLLVRVRGGQLRGIRLKAPGGPVSAFLGIPFAEPPVGSRRFMPPEPKRPWSGVLDATTFQNVCYQYVDTLYPGFEGTEMWNPNRELSEDCLYLNVWTPYPRPASPTPVLIWIYGGGFYSGAASLDVYDGRFLAQVEGAVLVSMNYRVGTFGFLALPGSREAPGNVGLLDQRLALQWVQENIAAFGGDPMSVTLFGESAGAASVGMHILSLPSRSLFHRAVLQSGTPNGPWATVSAGEARRRATLLARLVGCPPGGAGGNDTELIACLRTRPAQDLVDHEWHVLPQESIFRFSFVPVVDGDFLSDTPEALINTGDFQDLQVLVGVVKDEGSYFLVYGVPGFSKDNESLISRAQFLAGVRIGVPQASDLAAEAVVLHYTDWLHPEDPTHLRDAMSAVVGDHNVVCPVAQLAGRLAAQGARVYAYIFEHRASTLTWPLWMGVPHGYEIEFIFGLPLDPSLNYTTEERIFAQRLMKYWTNFARTGDPNDPRDSKSPQWPPYTTAAQQYVSLNLKPLEVRRGLRAQTCAFWNRFL

SEQ ID NO: 5 - protein, 63 aa, Aedes aegypti
ISVQQWNSYSGILGFPSAPTIDGVFMTADPMTMLRVEILVGSNRDEGTYFLLYDFIDYFEKDA

SEQ ID NO: 6 - protein, 61 aa, Anopheles gambiae
LVNNEWGTLGICEFPFVPVVDGAFLDETPQRSLATEILTGSNTEEGYYFIIYYLTELLRKE

SEQ ID NO: 7 - protein, 61 aa, Anopheles gambiae; identical to SEQ ID NO: 6

SEQ ID NO: 8 - protein, 63 aa, Anopheles stephensi
ISVQQWNSYSGILGFPSAPTIDGVFMTADPMTMLRIDILVGSNRDEGTYFLLYDFIDYFEKDA

SEQ ID NO: 9 - protein, 61 aa, Aphis gossypii
MVEKEWDHVAMCFFPFVPVVDGAFLDDHPQKSLSTNILMGSNSEEGYYFIFYYLTELFKKE

SEQ ID NO: 10 - protein, 62 aa, Aphis mellifera
LVKNEWGTLGICEFPFVPVIDGAFLDETPQRSLAANIMMGSNTEEGFYFIIYYLTELFHIDG

SEQ ID NO: 11 - protein, 63 aa, Bactrocera dorsalis
ISVQQWNSYSGILSFPSAPTIDGAFLPDHPMKMMEYDILMGNVRDEGTYFLLYDFIDYFDKDE

SEQ ID NO: 12 - protein, 63 aa, Bactrocera oleae; identical to SEQ ID NO: 11

SEQ ID NO: 13 - protein, 63 aa, Bemisia tabaci
VSSQQWSSYFGILGFPSAPTIDGEFLPKHPLELMKIELLIGSNRDEGTYFLLYDFLEFFEKDG

SEQ ID NO: 14 - protein, 61 aa, Blattella germanica
LVNNEWGTLGICEFPFVPIIDGTILDGPPQRSLATNILMGSNTEEGYYFIIYYLTELFRKE

SEQ ID NO: 15 - protein, 61 aa, Bombyx mori
LVNNEWGTLGICEFPFVPIIDGSFLDEMPVRSLATNILMGSNTEEGYYFILYYLTELFPKE

SEQ ID NO: 16 - protein, 61 aa, Culex pipiens
LVDNEWGTLGICEFPFVPVVDGAFLDETPQRSLATDILTGSNTEEGYYFIIYYLTELLRKE

SEQ ID NO: 17 - protein, 61 aa, Culex tritaeniorhynchus
LVDNEWGTLGICEFPFVPVVDGAFLDETPQRSLATEILTGSNTEEGYYFIIYYLTELLRKE

SEQ ID NO: 18 - protein, 61 aa, Cydia pomonella
LVNNEWGTLGICEFPFVPIIDGSFLDEMPIRSLATNLLLGSNTEEGYYFILYYLTELFPKE

SEQ ID NO: 19 - protein, 63 aa, Drosophila melanogaster
ISVQQWNSYSGILSFPSAPTIDGAFLPADPMTLMKYDILMGNVRDEGTYFLLYDFIDYFDKDD

SEQ ID NO: 20 - protein, 63 aa, Haematobia irritans
ISVQQWNSYSGILSFPSAPTIDGAFLPADPMTLLKYDILMGNVKDEGTYFLLYDFIDYFDKDD

SEQ ID NO: 21 - protein, 61 aa, Helicoverpa armigera
LVNNEWGTLGICEFPFVPIIDGSFLDELPVRSLVTNILMGSNTEEGYYFILYYLTELFPKE

SEQ ID NO: 22 - protein, 63 aa, Helicoverpa assulta
ISVQQWNSYTGILGFPSAPTVDGVFLPKDPDTMMKTEVLLGSNQDEGTYFLLYDFLDYFEKDG

SEQ ID NO: 23 - protein, 63 aa, Leptinotarsa decemlineata
ISLQQWNSYSGILGFPSTPTIEGVLLPKHPMDMLAMEILLGSNHDEGTYFLLYDFIDFFEKDG

SEQ ID NO: 24 - protein, 63 aa, Lucilia cuprina
ISVQQWNSYSGILSFPSAPTIDGAFLPADPMTLMKYDIMIGNVKDEGTYFLLYDFIDYFDKDE

SEQ ID NO: 25 - protein, 63 aa, Musca domestica
ISVQQWNSYSGILSFPSAPTIDGAFLPADPMTLLKYDILIGNVKDEGTYFLLYDFIDYFDKDD

SEQ ID NO: 26 - protein, 61 aa, Myzus persicae
MVEKEWDHVAICFFPFVPVVDGAFLDDYPQKSLSTNILMGSNSEEGYYSIFYYLTELFKKE

SEQ ID NO: 27 - protein, 63 aa, Nephotetix cincticeps
ISVQQWNSYFGILGFPSAPTIDGVFLPKHPLDLLKTEILIGSNQDEGTYFILYDFIDYFEKDG

SEQ ID NO: 28 - protein, 63 aa, Nilaparvata lugens
ISVQQWNSYSGILGLPSAPTIDGIFLPKHPLDLLKTEILIGSNQDEGTYFILYDFIDFFQKDG

SEQ ID NO: 29 - protein, 61 aa, Oulema oryzae
LVNNEAGTLGICDFPFVPVIDGAFLDEHPVRALATNILLGSNTEEGNYFIFYYLTELYKLE

SEQ ID NO: 30 - protein, 61 aa, Plutella xylostella
LVNNEWGTLGICEFPFVPIIDGSFLDEMPIRSLATNILMGSNTEEGYYFILYYLTELFPKE

SEQ ID NO: 31 - protein, 61 aa, Rhopalosiphum padi
MVEKEWDHVAICFFPFVPVVDGAFLDDHPQKSLSTNILMGSNSEEGYYSIFYYLTELFKKE

SEQ ID NO: 32 - protein, 61 aa, Schizaphis graminum; identical to SEQ ID NO: 31

SEQ ID NO: 33 - protein, 61 aa, Sitobion avenae; identical to SEQ ID NO: 26

SEQ ID NO: 34 - protein, 61 aa, Spodoptera exigua
LVNNEWGTLGICEFPFVPIIDGSFLDELPARSLATNLLMGSNTEEGYYYILYYLTELFPKE

SEQ ID NO: 35 - protein, 63 aa, Trialeurodes vaporariorum
VSSQQWSSYFGILGFPSAPTIDGVFLPKHPLELMKIEILIGSNRDEGTYFLLYDFLEFFEKDG

SEQ ID NO: 36 - protein, 64 aa, Bos taurus
LVDHEWRVLPQEHVFRFSFVPVVDGDFLSDTPEALINLQVLVGVVKDEGSYFLVYGAPGFSKDN

SEQ ID NO: 37 - protein, 64 aa, Canis familiaris
LVDHEWHVLPQESVFRFSFVPVVDGDFLSDTPEALISLQGLVGVVKDEGSYFLVYGAPGFSKDN

SEQ ID NO: 38 - protein, 64 aa, Felis catus
LVDHEWHVLPQESVFRFSFVPVVDGDFLSDTPEALINLQVLVGVVKDEGSYFLVYGAPGFSKDN

SEQ ID NO: 39 - protein, 64 aa, Homo sapiens
LVNHEWHVLPQESVFRFSFVPVVDGDFLSDTPEALINLQVLVGVVKDEGSYFLVYGAPGFSKDN

SEQ ID NO: 40 - protein, 64 aa, Macaca mulatta
LVNNEWHVLPQESVFRFSFVPVVDGDFLSDTPEALINLQVLVGVVKDEGSYFLVYGAPGFSKDN

SEQ ID NO: 41 - protein, 64 aa, Mus musculus
LVDHEWHVLPQESIFRFSFVPVVDGDFLSDTPEALINLQVLVGVVKDEGSYFLVYGVPGFSKDN

SEQ ID NO: 42 - protein, 64 aa, Oryctolagus cuniculus
LVDHEWRVLPQESIFRFSFVPVVDGDFLSDTPEALINLQVLVGVVKDEGTYFLVYGAPGFSKDN

SEQ ID NO: 43 - protein, 64 aa, Rattus norvegicus
LVDHEWHVLPQESIFRFSFVPVVDGDFLSDTPDALINLQVLVGVVKDEGSYFLVYGVPGFSKDN

SEQ ID NO: 44 - protein, 61 aa, Anopheles gambiae; identical to SEQ ID NO: 6

SEQ ID NO: 45 - protein, 61 aa, Anopheles gambiae; identical to SEQ ID NO: 6

SEQ ID NO: 46 - protein, 61 aa, Aphis gossypii; identical to SEQ ID NO: 9

SEQ ID NO: 47 - protein, 62 aa, Aphis mellifera; identical to SEQ ID NO: 10

SEQ ID NO: 48 - protein, 61 aa, Blattella germanica; identical to SEQ ID NO: 14

SEQ ID NO: 49 - protein, 61 aa, Bombyx mori; identical to SEQ ID NO: 15

SEQ ID NO: 50 - protein, 61 aa, Branchiostoma floridae
ISDNEWVVWGLCQFPFAPIVDGNFIREHPTQSLQTDVLVGFNNDEGVYFLLYGAPGFSKDT

SEQ ID NO: 51 - protein, 61 aa, Branchiostoma lanceolatum
ISENEWVVWGLCQFPFAPVVDGNFIREHPTVSLQTDVMVGFNNDEGVYFLLYGAPGFSKDT

SEQ ID NO: 52 - protein, 61 aa, Culex pipiens; identical to SEQ ID NO: 16

SEQ ID NO: 53 - protein, 61 aa, Culex tritaeniorhynchus; identical to SEQ ID NO: 17

SEQ ID NO: 54 - protein, 61 aa, Cydia pomonella; identical to SEQ ID NO: 18

SEQ ID NO: 55 - protein, 61 aa, Helicoverpa armigera; identical to SEQ ID NO: 21

SEQ ID NO: 56 - protein, 61 aa, Myzus persicae; identical to SEQ ID NO: 26

SEQ ID NO: 57 - protein, 61 aa, Oulema oryzae; identical to SEQ ID NO: 29

SEQ ID NO: 58 - protein, 61 aa, Plutella xylostella; identical to SEQ ID NO: 30

SEQ ID NO: 59 - protein, 61 aa, Rhopalosiphum padi; identical to SEQ ID NO: 31

SEQ ID NO: 60 - protein, 61 aa, Schizaphis graminum; identical to SEQ ID NO: 31

SEQ ID NO: 61 - protein, 61 aa, Sitobion avenae; identical to SEQ ID NO: 26

SEQ ID NO: 62 - protein, 61 aa, Spodoptera exigua; identical to SEQ ID NO: 34

SEQ ID NO: 63 - protein, 63 aa, Aedes aegypti; identical to SEQ ID NO: 5

SEQ ID NO: 64 - protein, 63 aa, Anopheles gambiae; identical to SEQ ID NO: 8

SEQ ID NO: 65 - protein, 63 aa, Anopheles stephensi; identical to SEQ ID NO: 8

SEQ ID NO: 66 - protein, 63 aa, Aphis gossypii
ISKKQWNSYSGILGFPSAPTVDGILLPEHPLDMLAIDILIGSNLNEGTYFLLYDFVDFFDRTS

SEQ ID NO: 67 - protein, 63 aa, Apis mellifera
ISVQQWNSYWGILGFPSAPTIDGIFLPKHPLDLLRTEILIGNNENEGTYFILYDFNDIFEKDQ

SEQ ID NO: 68 - protein, 63 aa, Bactrocera dorsalis; identical to SEQ ID NO: 11

SEQ ID NO: 69 - protein, 63 aa, Bactrocera oleae; identical to SEQ ID NO: 11

SEQ ID NO: 70 - protein, 63 aa, Bemisia tabaci; identical to SEQ ID NO: 13

SEQ ID NO: 71 - protein, 63 aa, Blattella germanica
ISVKQWNSYWGILGFPSAPTIDGVFLPKHPMDLIKTEILIGSNQDEGTYFILYDFMDYFEKDS

SEQ ID NO: 72 - protein, 63 aa, Bombyx mori
ISVQQWNSYTGILGFPSAPTVDGIFLPKDPDTMMKSEVLLGSNQDEGTYFLLYDFLDYFEKDG

SEQ ID NO: 73 - protein, 61 aa, Boophilus decoloratus
IVNNETNSGGVVDFPFVPVVDGVFLPDTPQTLMDISVMLGSNANEGSWFLQYFFGFPVSDE

SEQ ID NO: 74 - protein, 61 aa, Boophilus microplus
IVNNETNSGGVVDFPFVPVADGVFLPDTPQALTDISVMLGSNANEGSWFLQYFFGFPVTDE

SEQ ID NO: 75 - protein, 63 aa, Boophilus microplus
IVAVEATFFGSGSGKFEPIYGDEFLPIEPRMADFPKDVMIGQTAYEGSNILYTTFRDTFSEAL

SEQ ID NO: 76 - protein, 62 aa, Boophilus microplus
LLIMSNTIFGVHALTFFPVFGDDIIPDDPYLMMEADLLIGNNLDEGSYFVFYLFGRALDLEQ

SEQ ID NO: 77 - protein, 64 aa, Bos taurus; identical to SEQ ID NO: 36

SEQ ID NO: 78 - protein, 67 aa, Branchiostoma floridae
ILDHEWNVVDLTGAHFLADIPFPPIKDGSFLTEDPTEVYEIDILVGFVKNEGNFWLVYGVPGFSKDT

SEQ ID NO: 79 - protein, 67 aa, Branchiostoma lanceolatum
ILDHEWNVVDLSDAHFLADIPFPPVKDGRFITEDPAEMYAIDILVGFVKDEGNFWLVYGVPGFDKDT

SEQ ID NO: 80 - protein, 64 aa, Bungarus fasciatus
LIDEEWSVLPYKSIFRFPFVPVIDGDFFPDTPEAMLSTQVLLGVVKDEGSYFLIYGLPGFSKDN

SEQ ID NO: 81 - protein, 63 aa, Caenorhabditis briggsae
LRENEWAPVREFGDFPWVPVVDGDFLLENAQTSLKTQLLAGSNRDESIYFLTYQLPDIFPVAD

SEQ ID NO: 82 - protein, 65 aa, Caenorhabditis briggsae
VQAEADNISGDIGPPMTFAYVPVSSDANFFQGDVIQKVNIIFGSVKDEGTYWLPYYMSLPKYGFA

SEQ ID NO: 83 - protein, 58 aa, Caenorhabditis briggsae
LIDNIWNVGLNFLEFPFAIVSKDRNFFKHLDGFIAVNLMFGINHDEGNFWNIYNLAKF

SEQ ID NO: 84 - protein, 58 aa, Caenorhabditis briggsae
LSLSTWNISLTYLEFPFVIVSRDKHFFGHLDARAAVNLMIGMNKDEGNYWNIYQLPQF

SEQ ID NO: 85 - protein, 63 aa, Caenorhabditis elegans; identical to SEQ ID NO: 81

SEQ ID NO: 86 - protein, 65 aa, Caenorhabditis elegans
VQAEADNISGDIGPPMTFAYVPVSSDANFFQGDVFQKVNIIFGSVKDEGTYWLPYYMSLPKYGFA

SEQ ID NO: 87 - protein, 58 aa, Caenorhabditis elegans
LIDNIWNVGLNFLEFPFAIVSKDQNFFKHLDGFIAVNLMFGINHDEGNFWNIYNLAKF

SEQ ID NO: 88 - protein, 58 aa, Caenorhabditis elegans
LSLSTWNISLTYLEFPFVIVSRDKHFFGHLDAHAAVNLMIGMNKDEGNYWNIYQLPQF

SEQ ID NO: 89 - protein, 64 aa, Canis familiaris; identical to SEQ ID NO: 37

SEQ ID NO: 90 - protein, 64 aa, Carassius auratus
LIDQEWQVLPYSSLFRFSFVPVIDGVFLPDTPEAMINTQILLGVNQDEGSYFLLYGAPGFSKDN

SEQ ID NO: 91 - protein, 57 aa, Ciona intestinalis
VFSNSWITQEIFDFPFVPVHGTSFLPEHPHEVTRVDVMAGHNTNEGSYFTLYTVPGF

SEQ ID NO: 92 - protein, 57 aa, Ciona savignyi
VFAGSWITKEIFDFPFVPVHGTTFLPEHPHEVTRVDVLAGYNTNEGSYFTIYTVPGY

SEQ ID NO: 93 - protein, 47 aa, Culex pipiens
ISVQQWNSYSGILGFPSAPTIDGVFMTADPMTMLRIDILVGSNRDEG

SEQ ID NO: 94 - protein, 63 aa, Culex tritaeniorhynchus; identical to SEQ ID NO: 8

SEQ ID NO: 95 - protein, 63 aa, Cydia pomonella; identical to SEQ ID NO: 22

SEQ ID NO: 96 - protein, 64 aa, Danio rerio
LIDQEWQVLPWSSLFRFSFVPVVDGVFFPDTPDAMISTQILLGVNQDEGSYFLLYGAPGFSKDN

SEQ ID NO: 97 - protein, 61 aa, Dermacentor variabilis
IVKSEPCSGGVVDFPFAPVEDGAFLPGTPQALMDISVMLGSNVNEGSWFLQYFFGLSVTEE

SEQ ID NO: 98 - protein, 62 aa, Dictyocaulus viviparus
ILTAGSAMFDLEPPMSFPFVPVDEDKVFFNGNVRDIVPAVMGTVNDEGTFWLPYYLKETGFT

SEQ ID NO: 99 - protein, 62 aa, Dictyocaulus viviparus
ILNAGGAMFYLEPPMSFPFVPVDEDKVFFNGNVRDIVPAVMGTVNDEGTFWLPYYLKETGFT

SEQ ID NO: 100 - protein, 62 aa, Dictyocaulus viviparus
VQSAGYAMFGLEPPMSFPFVPVDEDKVFFNGNVRDIVPAVMGTVNDEGTFWLPYYLGDTGFA

SEQ ID NO: 101 - protein, 63 aa, Dictyocaulus viviparus
IQAEADYVSNDIGLPMTFAFVPVSADKNFFKGNVFERVAVIFGTVKDEGTYWLPYYMSRYGFW

SEQ ID NO: 102 - protein, 63 aa, Drosophila melanogaster; identical to SEQ ID NO: 19

SEQ ID NO: 103 - protein, 64 aa, Electrophorus electricus
LIDQEWLVLPFSGLFRFSFVPVIDGVVFPDTPEAMLNTQILLGVNQNEGSYFLIYGAPGFSKDN

SEQ ID NO: 104 - protein, 64 aa, Felis catus; identical to SEQ ID NO: 38

SEQ ID NO: 105 - protein, 64 aa, Fugu rubripes
LLNQEFKVLPWASIFRFPFVPVVDGDVLPDSPQAMISTQLLLGFNQDEGTYFLLYGAPGFSKDN

SEQ ID NO: 106 - protein, 63 aa, Fugu rubripes
LIIAQYGVLTEDGLGGYPFVPVVDGVFLTDHPQIMLDKDVLLGLNKDEGSYFMAYGLPEFELG

SEQ ID NO: 107 - protein, 61 aa, Fugu rubripes
LLTAQFGVLTGLGTSPFLPVVDGVFLPDTLEALLEMELLLGLNKNEGSYFLVYSFPGYDLG

SEQ ID NO: 108 - protein, 116 aa, Gallus gallus
VLEGEGVVMPPQSVFRFAFVPVVDGDFVVDSPDVALWGDYGVKGGEGGHGVEGGDGGGAKMPPRPHRDETSPDAYGAKMPPRPHRAGGEVEVLLGAVRVEGSYFLVYGVPGFGKDN

SEQ ID NO: 109 - protein, 63 aa, Haematobia irritans; identical to SEQ ID NO: 20

SEQ ID NO: 110 - protein, 63 aa, Helicoverpa armigera; identical to SEQ ID NO: 22

SEQ ID NO: 111 - protein, 63 aa, Helicoverpa assulta; identical to SEQ ID NO: 22

SEQ ID NO: 112 - protein, 64 aa, Homo sapiens; identical to SEQ ID NO: 39

SEQ ID NO: 113 - protein, 63 aa, Leptinotarsa decemlineata; identical to SEQ ID NO: 23

SEQ ID NO: 114 - protein, 62 aa, Loligo opalescens
FPDHEWWVIQGISQFPFVLVVDGTFLVEAPEIALEVPILVGTNKNEGTYFLTYFRQDIFNLK

SEQ ID NO: 115 - protein, 63 aa, Lucilia cuprina; identical to SEQ ID NO: 24

SEQ ID NO: 116 - protein, 64 aa, Macaca mulatta; identical to SEQ ID NO: 40

SEQ ID NO: 117 - protein, 63 aa, Meloidogyne incognita
LRDSEWSPVMEFADFPWVPVIDGEFLVENIETSLKTQLLAGSNFDEAIYFIVYQLADVFPPAE

SEQ ID NO: 118 - protein, 63 aa, Meloidogyne incognita
IQRAGDAVSQSLSLPMDFAFVPIDEDTHFFRGNVFDKVSILVGTVRDEGTYWLPYCLQKNGFG

SEQ ID NO: 119 - protein, 63 aa, Meloidogyne javanica
LRDSEWSPVMEFADFPWVPVIDGEFLVENIETSLKTQLLAGSNFDEAIYFIVYQLSDVFPPAE

SEQ ID NO: 120 - protein, 64 aa, Mus musculus; identical to SEQ ID NO: 41

SEQ ID NO: 121 - protein, 63 aa, Musca domestica; identical to SEQ ID NO: 25

SEQ ID NO: 122 - protein, 64 aa, Myxine glutinosa
IVSKEGDVVIEPSIFRFPFVPVVDGHFIIDSPIVLLQTDLLLGVNRNEGSFFLIYGAPGFSKDH

SEQ ID NO: 123 - protein, 63 aa, Myzus persicae
ISKKQWNNYSGILGFPSAPTVDGVLLPEHPLDMLAIDILIGSNLNEGTYFLLYDFVDFFDRTS

SEQ ID NO: 124 - protein, 63 aa, Necator americanus
VLKEAAVVSYQIGLVLTFAFIPITSDKNFFQGNVFDRVSIVLGTVKDEATFFLPYYFGHNGFS

SEQ ID NO: 125 - protein, 63 aa, Nephotetix cincticeps; identical to SEQ ID NO: 27

SEQ ID NO: 126 - protein, 63 aa, Nilaparvata lugens; identical to SEQ ID NO: 28

SEQ ID NO: 127 - protein, 63 aa, Nippostrongylus brasiliensis
IQNISDTMYEHMRPMEWPFGPITGDVNFFQGRCTTKKVSAIFGTVKDEGTFWLPYYLYDSGFA

SEQ ID NO: 128 - protein, 64 aa, Nippostrongylus brasiliensis
IQSEGNSDAIYAEMLPMEWPFGPITYDDNYFKGEVRRKVSAIFGTVKDEGTFWLPYYLSESGFS

SEQ ID NO: 129 - protein, 64 aa, Nippostrongylus brasiliensis
IQSAGNSDAIYENMLPMEWPFGPITYDDNYFKGDVRRKVSAIFGTVKDEGTFWLPYYLSDSGFS

SEQ ID NO: 130 - protein, 64 aa, Oryctolagus cuniculus; identical to SEQ ID NO: 42

SEQ ID NO: 131 - protein, 64 aa, Oryzias latipes
ITAKQYDIITEPPLLNFPFGPTVDGVFLTAEVEKLLTKELMIGLNKDEGTCFLVYGSPGFSITG

SEQ ID NO: 132 - protein, 63 aa, Plutella xylostella; identical to SEQ ID NO: 22

SEQ ID NO: 133 - protein, 64 aa, Rattus norvegicus; identical to SEQ ID NO: 43

SEQ ID NO: 134 - protein, 61 aa, Rhipicephalus appendiculatus
IVNNETNSGGVIDFPFVPVVDGVLLPDTPQTLMDISVMLGSNANEGSWFLQYFFGLPVTEE

SEQ ID NO: 135 - protein, 61 aa, Rhipicephalus sanguineus
IVNNETNSGGVVDFPFVPVVDGVFLRDTPQALMDISVMLGSNLNEGSWFLQYFFGLPATEE

SEQ ID NO: 136 - protein, 63 aa, Rhopalosiphum padi
ISKKQWNSYSGILGFPSAPTVDGIFLPEHPLDMLAIDILIGSNLNEGTYFLLYDFVDFFDRTS

SEQ ID NO: 137 - protein, 65 aa, Schistosoma bovis
ILDAHDTMYDPASYFSVPFPPVLDNNFFPYENSQSFRGALMFGINKNEGSYFLLYAFVSNSKWMK

SEQ ID NO: 138 - protein, 65 aa, Schistosoma haematobium; identical to SEQ ID NO: 137

SEQ ID NO: 139 - protein, 65 aa, Schistosoma mansoni; identical to SEQ ID NO: 137

SEQ ID NO: 140 - protein, 63 aa, Sitobion avenae
ISKKQWNSYSGILGFPSAPTVDGVLLPEHPLDMLAIDILIGSNLNEGTYFLLYDFVDFFDRTS

SEQ ID NO: 141 - protein, 53 aa, Tetranychus cinnabarinus
FNLIAEFYPVMGDEFLPSEPEKLIKSNILLGVVGNEGVGLFRYLRFDERFFNP

SEQ ID NO: 142 - protein, 61 aa, Tetranychus kanzawai
LVAQEEATTGVVEFAFIPIVDGSFLDEDPEVSLRTPILTGSNRDEGTYFLVYHSPHIFNLS

SEQ ID NO: 143 - protein, 61 aa, Tetranychus urticae
LVAQEETTTGVVEFAFIPIVDGSFLDEDPEVSLRTPILTGSNRDEGTYFLVYHSPHIFNLS

SEQ ID NO: 144 - protein, 64 aa, Tetraodon nigroviridis
LINQEWKVLPWPALFRFSFVPVVDGDVLPDSPQAMISTQLLLGFNQDEGSYFLLYGAPGFSKDN

SEQ ID NO: 145 - protein, 60 aa, Tetraodon nigroviridis
LAISQFAVLDSGGYPFFPVVDGVFLPDKPEVMIKKELLIGVNKDEGSYFMEYGLPGFDLG

SEQ ID NO: 146 - protein, 61 aa, Tetraodon nigroviridis
LAVAQFGVLSNLESYPFFPVVDGVFLPDTPKALLNKELLLGVNKNEASYFLVYAVPGYDLG

SEQ ID NO: 147 - protein, 64 aa, Tetraodon nigroviridis
LLKVQYGVLSNPSTSDIPFLPVVDGVFLPDEIDALISKEVLLGLNHDEGTYFLVYTVPGFDITS

SEQ ID NO: 148 - protein, 53 aa, Tetraodon nigroviridis
LEVFETWGPYIDGDLIKEQAVTAFQKGSVLLGTTSEEGVIFVYSVFNKPVSAV

SEQ ID NO: 149 - protein, 64 aa, Torpedo californica
LIDVEWNVLPFDSIFRFSFVPVIDGEFFPTSLESMLNTQILLGVNKDEGSFFLLYGAPGFSKDS

SEQ ID NO: 150 - protein, 64 aa, Torpedo marmorata; identical to SEQ ID NO: 149

SEQ ID NO: 151 - protein, 63 aa, Trialeurodes vaporariorum; identical to SEQ ID NO: 35

SEQ ID NO: 152 - protein, 64 aa, Xenopus tropicalis
LIDHEFSVLPAPSVFRFAFVPVPDADFFPEPPEILMNCPLIMGVNQNEGSYFLLYGAPGFSKNN

(19) United States
(12) Patent Application Publication
(10) Pub. No.: US 2010/0168114 A1
(43) Pub. Date: Jul. 1, 2010

(54) INVERTEBRATE ACETYLCHOLINESTERASE INHIBITORS

(76) Inventor: Yuan-Ping Pang, Rochester, MN (US)

Correspondence Address: FISH & RICHARDSON P.C., PO BOX 1022, MINNEAPOLIS, MN, 55440-1022, US

(21) Appl. No.: 12/641,900

(22) Filed: Dec. 18, 2009

Related U.S. Application Data
(63) Continuation-in-part of application No. PCT/US2008/067236, filed on Jun. 17, 2008.
Application No. PCT/US2008/067236 is a continuation-in-part of application No. 11/764,580, filed on Jun. 18, 2007, now abandoned.

(51) Int. Cl. A01N 43/707; C07D 311/06; A01N 43/16; C07D 405/02; A01N 43/54; C07D 215/00; A01N 43/42; C07D 239/70; A01N 43/90; C07D 473/00; C07D 253/06; A01N 43/60; A01P 7/04; A01P 15/00; C12Q 1/46; C07C 211/62; C07D 213/72; G06F 19/00

(52) U.S. Cl. 514/242; 549/289; 514/456; 544/333; 514/256; 546/156; 514/312; 544/282; 514/259.41; 544/276; 514/252.16; 544/182; 544/379; 514/254.1; 544/371; 514/254.06; 435/20; 564/291; 546/304; 702/19

(57) ABSTRACT

Methods for determining invertebrate- and insect-specific, such as mosquito-specific, residues of acetylcholinesterases are provided herein. The methods can be used to design pesticides and insecticides that are specific for the invertebrate or insect (e.g., mosquito) enzymes, resulting in reduced toxicity concerns for mammals. Compositions for inhibiting invertebrate and insect (e.g., mosquito) acetylcholinesterases and methods for preparing the same are also provided.

### CROSS-REFERENCE TO RELATED APPLICATION

This application is a continuation-in-part application and claims priority to PCT Application Ser. No. PCT/US2008/067236, filed Jun. 17, 2008, and U.S. application Ser. No. 11/764,580, filed Jun. 18, 2007, incorporated by reference in its entirety herein.

### TECHNICAL FIELD

This invention relates to invertebrate acetylcholinesterase inhibitors, including insect, such as mosquito, acetylcholinesterase inhibitors, and methods for identifying the same, and more particularly to the use of a refined 3D model of the Anopheles gambiae acetylcholinesterase enzyme in the design and selection of invertebrate- and insect-specific acetylcholinesterase inhibitors.

### BACKGROUND

Acetylcholinesterase (AChE), a serine hydrolase vital for regulating the neurotransmitter acetylcholine in mammals and in insects, has long been used as a target for pesticides. The enzyme has a deep and narrow active site, the bottom and opening regions of which are known as catalytic and peripheral sites, respectively. Current anticholinesterase pesticides for controlling pests, including the African malaria-carrying mosquito (Anopheles gambiae), were developed during the World War II era. These pesticides are toxic to mammals because they target a catalytic serine residue of acetylcholinesterases (AChEs) that is present in both insects and in mammals, and thus the use of these pesticides has been severely limited. Although it has long been assumed that humans are not harmed by low applications of the anticholinesterase inhibitors, as pests are more sensitive to the chemicals than humans, a recent report by the U.S. Environmental Protection Agency's Office of Inspector General indicates that some anticholinesterase inhibitors can enter the brain of fetuses and young children and may destroy cells in the developing nervous system. The use of anticholinesterase-targeted pesticides has also been limited by resistance problems of mosquitoes possessing AChE mutants, such as the G119S mutant that is insusceptible to current pesticides.

### SUMMARY

Recent outbreaks of locally acquired mosquito-transmitted malaria in the United States demonstrate the continued risk for reintroduction of the disease. The present disclosure is directed to, among other things, materials and methods for controlling, e.g., selectively killing, insect populations, e.g., to control insect-borne diseases and to limit or stop crop damage mediated by insects. The present disclosure identifies conserved AChE target sites that are present in invertebrate, including insect such as mosquito, AChEs. Such regions can be used as better target sites for the design of new pesticides that would be devoid of the mammalian toxicity and resistance problems of current pesticides. As disclosed herein, a sequence analysis of AChEs from 73 species that are currently publicly available and a 3D model of AgAChE generated by homology modeling and refinement with multiple molecular dynamics simulations revealed two conserved residues (C286 and R339) present at the opening of the active site of AgAChE but absent at those of mammalian AChEs. While a three-dimensional (3D) model of African malaria-carrying mosquito (Anopheles gambiae) AChE (AgAChE) has been reported [6], no conserved and mosquito-specific region of AgAChE has previously been reported. Comparative sequence and structural analysis of the Anopheles gambiae AChE (AgAChE) 3D model reported here shows that a cysteine corresponding to the C286 of A. gambiae is present at the opening of the active site of AChEs in 17 invertebrate species, and that an arginine corresponding to A. gambiae's R339 is present at the opening of the active site of AChEs in 4 insect species. Both residues are not present in the active site of AChEs of human, monkey, dog, cat, cattle, rabbit, rat, and mouse. The 17 invertebrates having the cysteine include house mosquito, Japanese encephalitis mosquito, African malaria mosquito, German cockroach, Florida lancelet, rice leaf beetle, African bollworm, beet armyworm, codling moth, diamondback moth, domestic silkworm, honey bee, oat or wheat aphid, the greenbug, melon or cotton aphid, green peach aphid, and English grain aphid. The 4 insect species having the arginine are house mosquito, Japanese encephalitis mosquito, African malaria mosquito, and German cockroach. These 4 insect species have both the cysteine and arginine that correspond to the R339 and C286 of A. gambiae. The discovery of the two invertebrate-specific residues enables the design of effective and safer pesticides that target one or more of the invertebrate (insect, e.g., mosquito) AChE-specific residues, rather than the serine residue present in both insect and mammalian AChE enzymes, thus potentially offering an effective control of invertebrate-borne (e.g., insect-borne) diseases (e.g., mosquito-borne diseases such as malaria, encephalitis, and West Nile virus). In addition, methods of designing and synthesizing compositions for use in eliminating or controlling insect populations that contribute to significant crop damage, e.g., wheat, soybean, grain, sorghum, barley, oat, peach, or melon damage caused by insects such as aphids, are also described.

Accordingly, in one embodiment, a computer-assisted method of generating a test inhibitor of the acetylcholinesterase site activity of an invertebrate acetylcholinesterase (AChE) polypeptide is provided. The method uses a programmed computer comprising a processor and an input device and includes:

(a) inputting on the input device data comprising a structure of an acetylcholinesterase active site of an invertebrate AChE polypeptide;
(b) docking into the active site a test inhibitor molecule using the processor; and
(c) determining, based on the docking, whether the test inhibitor molecule would be capable of interacting with a residue of the invertebrate AChE polypeptide corresponding to Arg339 of AgAChE.

The acetylcholinesterase active site can have acetylcholine bound. The method can further include derivatizing the test inhibitor molecule so that the test inhibitor molecule is capable of interacting with a residue of the invertebrate AChE polypeptide corresponding to Cys286 of AgAChE. The method can also further include derivatizing the test inhibitor molecule so that the test inhibitor molecule is capable of interacting with a residue of the invertebrate AChE polypeptide corresponding to Trp84 of AgAChE.

The inhibitory activity of the test inhibitor can be evaluated on an invertebrate or mammalian AChE polypeptide in vitro. In some embodiments, the invertebrate AChE polypeptide can be selected from Anopheles gambiae AChE, Culex pipiens AChE, or Culex tritaeniorhynchus AChE.

In another embodiment, a method of generating a compound that inhibits the acetylcholinesterase site activity of the Anopheles gambiae AChE polypeptide is provided which includes:

(a) providing a three-dimensional structure of the Anopheles gambiae AChE polypeptide acetylcholinesterase active site; and

(b) designing, based on the three-dimensional structure, a test compound capable of interacting with Arg339.

In some embodiments, the test compound is further capable of interacting with Cys286. In some embodiments, the test compound is further capable of interacting with W84. In yet other embodiments, the test compound is further capable of interacting with both Cys286 and W84.

Compositions of matter are also provided herein. A composition of matter can include a compound according to any of Formula I, II, III, IV, V, VI, VII, or VIII described herein, or an acceptable salt or derivative thereof. Insecticidal and pesticidal compositions comprising a compound according to any of Formula I-VIII and a carrier are also provided.

Compounds for use in the compositions and methods provided herein, or acceptable salts or derivatives thereof, can be according to Formula I:

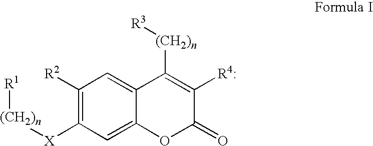

wherein n=0 to 3;
wherein R¹ is selected from:

a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

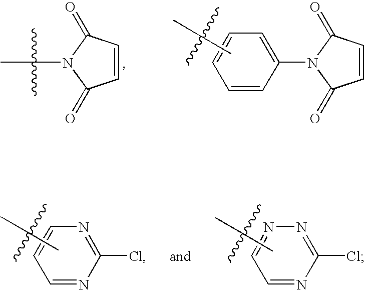

and X₅=alkyl(C₁-C₅) or —(O)₂alkyl(C₁-C₅); and

b) H and one of the moieties A, B, C, D, and E:

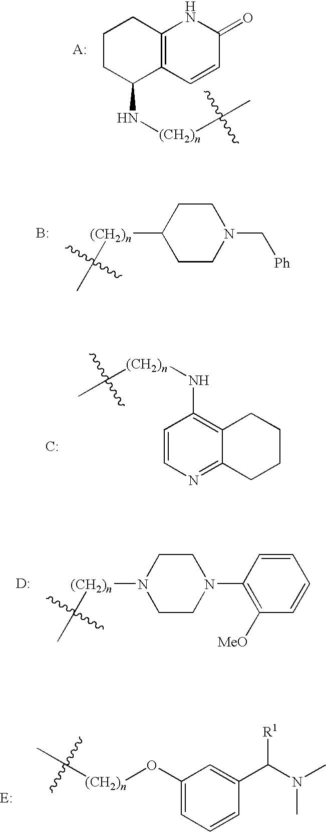

wherein E's R¹═H or Me; and n for A-E ranges from 0 to 5;

wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10, X₄ is as above, and X₅=alkyl(C₁-C₅) or —(O)₂alkyl(C₁-C₅);
wherein R³ is selected from H, CH₃, and

a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅, where n=1 to 10, X⁴ is as above, and X₅=alkyl(C₁-C₅) or —(O)₂alkyl(C₁-C₅); and

b) one of the moieties A, B, C, D, and E:

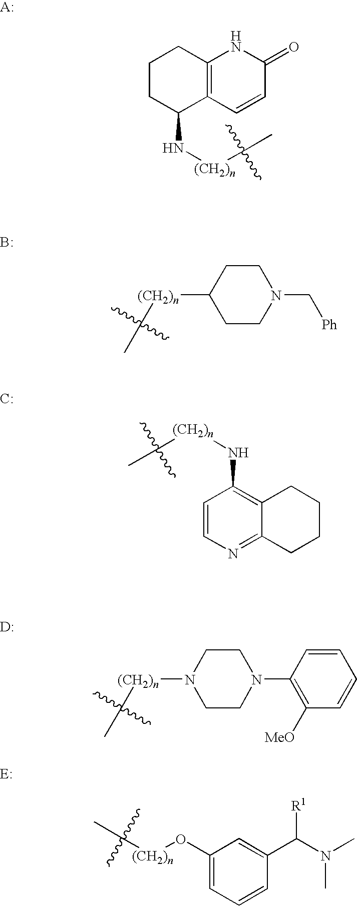

wherein E's R¹═H or Me; and n for A-E ranges from 0 to 5;

wherein R⁴ is selected from:

H and one of the moieties A, B, C, D, and E:

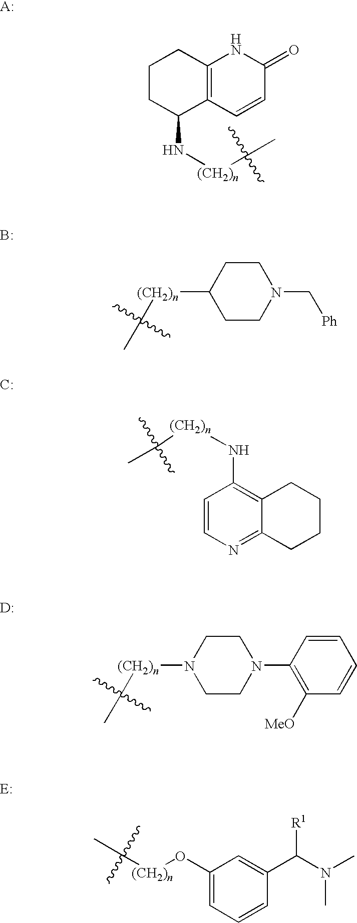

wherein E's R¹═H or Me; and n for A-E ranges from 0 to 5;

wherein X═O or NR⁵, wherein R⁵ is selected from H or C₁-C₅ alkyl.

In some embodiments, a compound according to Formula I is a compound according to Formula IA:

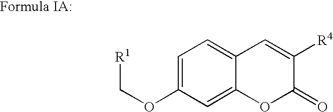

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH2)ₙS—SX₅ and H, where n=1 to 10; X₄ is selected from:

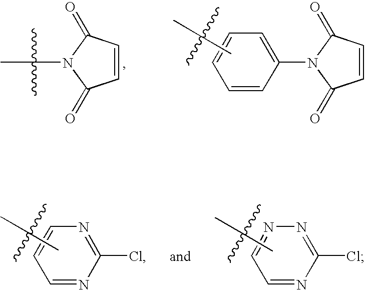

and X₅=alkyl(C₁-C₅) or —(O)₂alkyl(C₁-C₅); and

wherein R⁴ is selected from: H and one of the moieties A, B, C, D, and E:

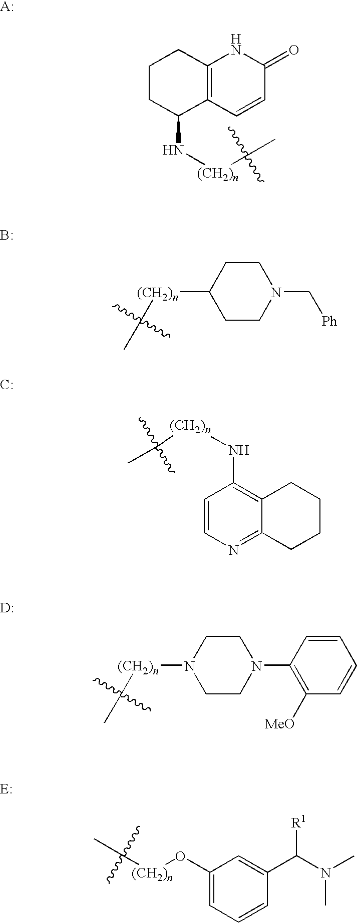

wherein E's R¹═H or Me; and n for A-E ranges from 0 to 5.

In some embodiments, a compound according to Formula I is according to Formula IB:

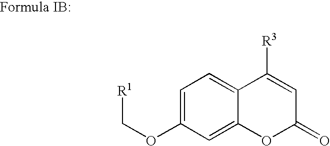

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

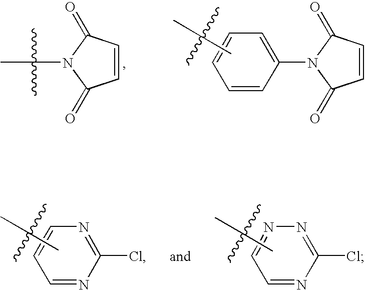

and X₅=alkyl(C₁-C₅) or —(O)₂alkyl(C₁-C₅); and

wherein R³ is selected from H and one of the moieties A, B, C, D, and E:

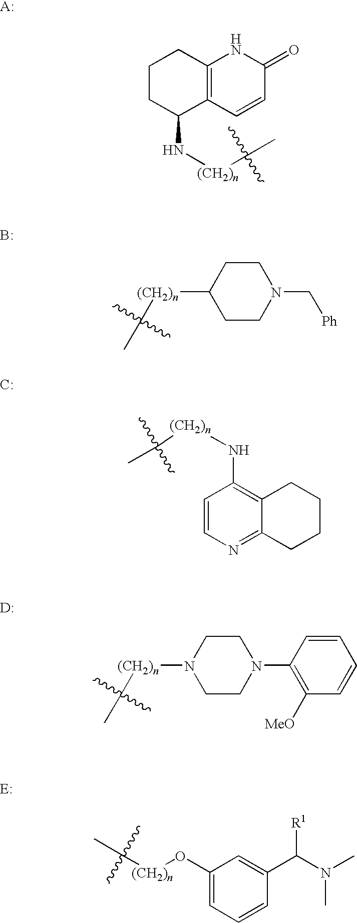

wherein E's R¹═H or Me; and n for A-E ranges from 0 to 5.

In some embodiments, a compound according to Formula I is according to Formula IC:

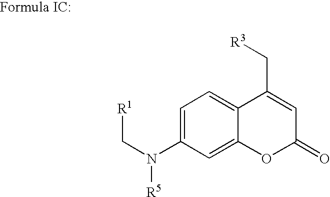

wherein R¹ is selected from H and one of the moieties A, B, C, D, and E:

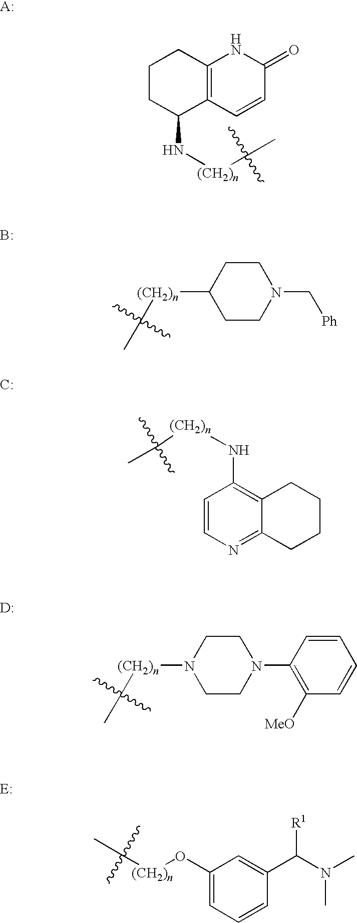

wherein E's R¹═H or Me; and n for A-E ranges from 0 to 5;

wherein R³ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

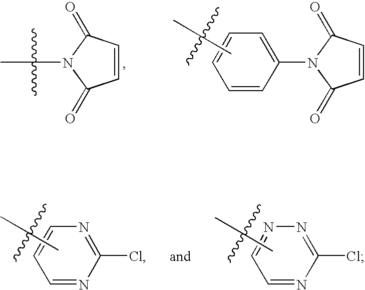

and X₅=alkyl(C₁-C₅) or —(O)₂alkyl(C₁-C₅); and

R⁵ is as above in Formula I.

In some embodiments, a compound according to Formula I is according to Formula ID:

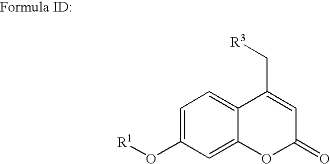

wherein R¹ is selected from selected from H and one of the moieties A, B, C, D, and E:

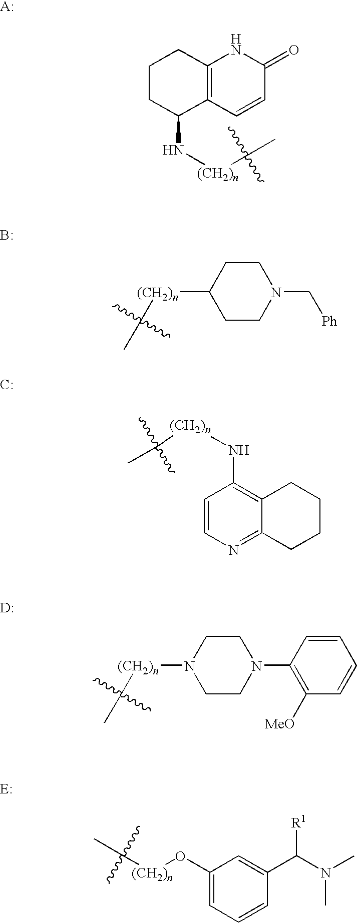

wherein E's R¹═H or Me; and n for A-E ranges from 0 to 5;

wherein R³ selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

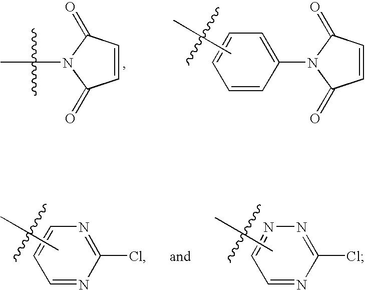

and X₅=alkyl(C₁-C₅) or —(O)₂alkyl(C₁-C₅).

In some embodiments, a compound according to Formula I is according to Formula IE:

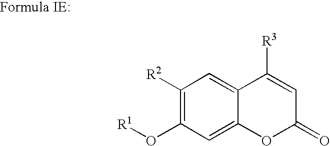

wherein R³ is H or CH₃;
wherein R¹ is selected from H and one of the moieties A, B, C, D, and E:

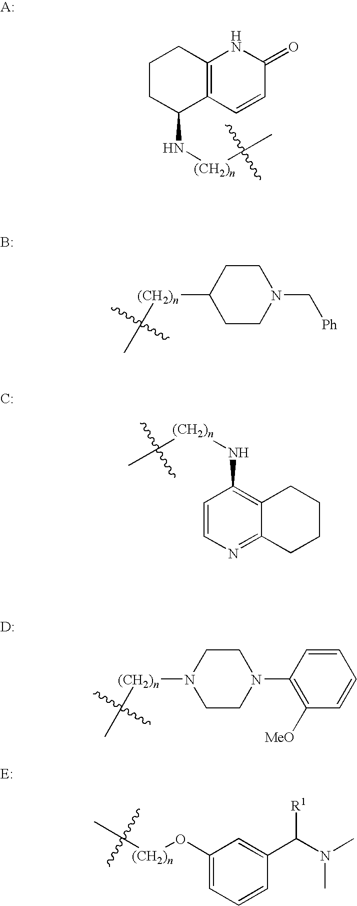

wherein E's R¹═H or Me; and n for A-E ranges from 0 to 5; and

wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

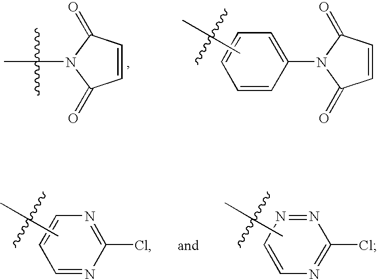

and X₅=alkyl(C₁-C₅) or —(O)₂alkyl(C₁-C₅).
In some embodiments, a compound for use in the present methods is according to Formula II:

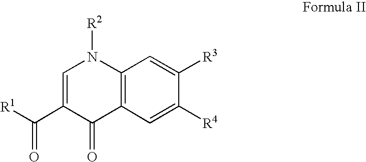

wherein R¹ is H, OMe, OEt, NH₂, alkyl(C₁-C₅), or is selected from: A, B, C, D, and E:

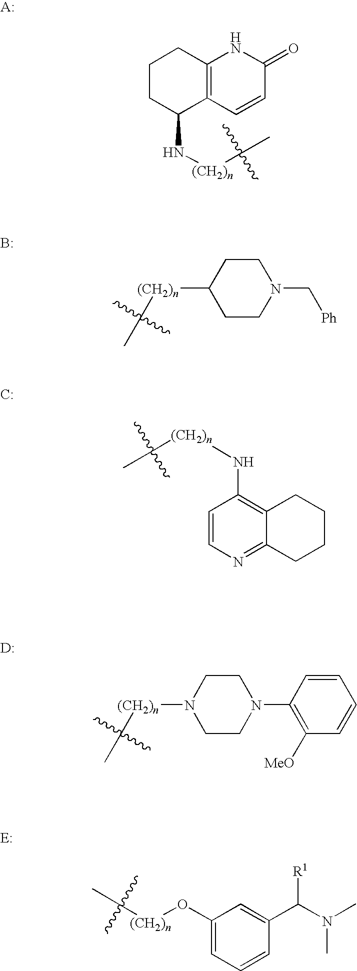

wherein E's R¹═H or Me; and n for A-E ranges from 0 to 5;
wherein R² and R³ are independently selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

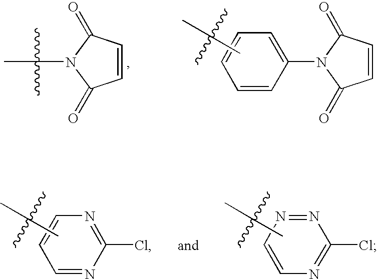

and X₅=alkyl(C₁-C₅) or —(O)₂alkyl(C₁-C₅);

wherein R⁴ is selected from: H and one of the moieties A, B, C, D, and E:

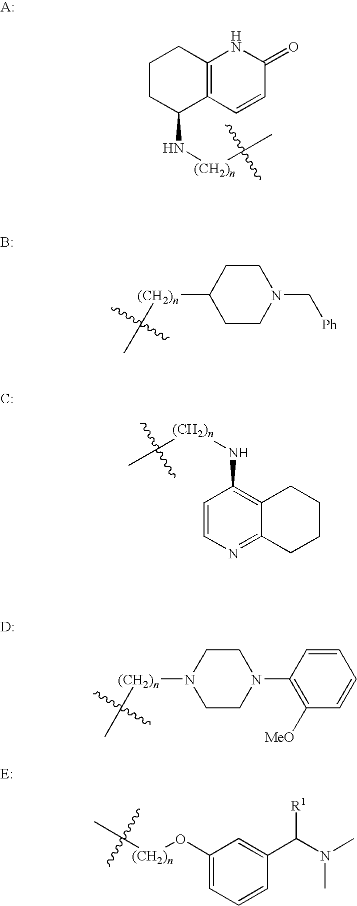

wherein E's R¹═H or Me; and n for A-E ranges from 0 to 5.

In some embodiments, a compound according to Formula II is according to Formula IIA or Formula IIB, below:

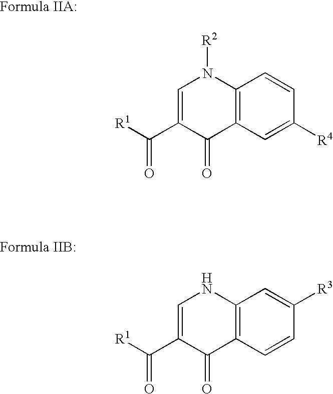

In some embodiments, a compound for use in the methods described herein is according to Formula IIIA or Formula IIIB:

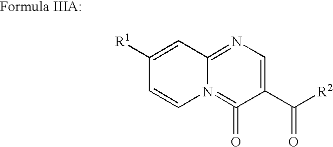

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

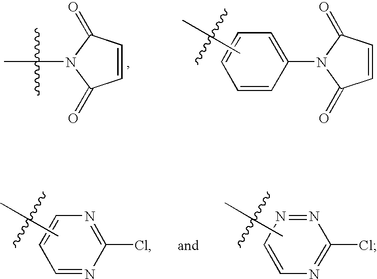

and X₅=alkyl(C₁-C₅) or —(O)₂alkyl(C₁-C₅); and

wherein R² is selected from: H and one of the moieties A, B, C, D, and E:

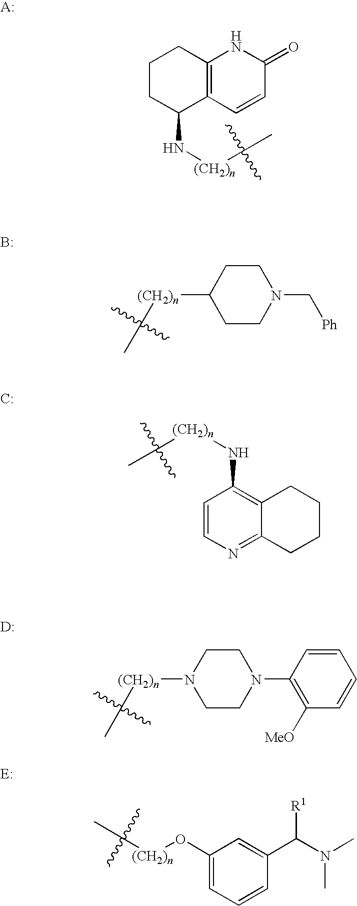

wherein E's R¹═H or Me; and n for A-E ranges from 0 to 5.

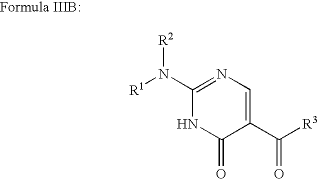

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

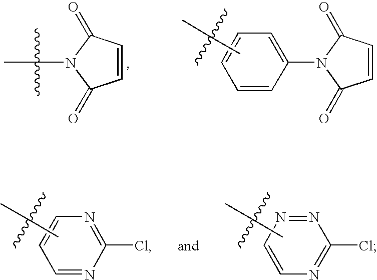

and X₅=alkyl(C₁-C₅) or —(O)₂alkyl(C₁-C₅); and

R² is selected from H and alkyl(C₁-C₅); and
R³ is selected from: H and one of the moieties A, B, C, D, and E:

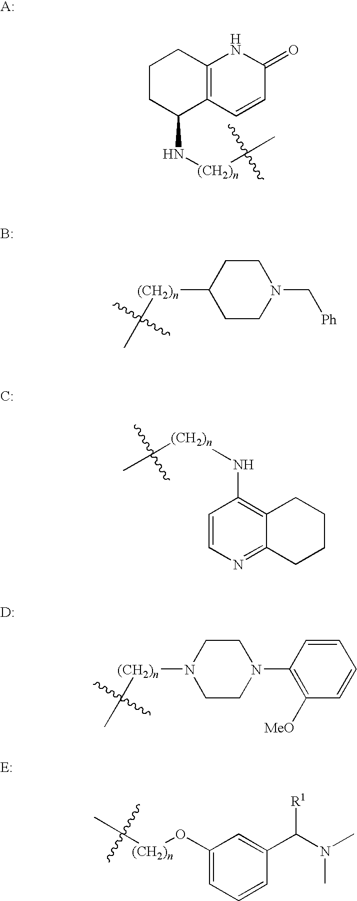

wherein E's R¹═H or Me; and n for A-E ranges from 0 to 5.

In some embodiments, a compound for use in the invention is according to Formula IV:

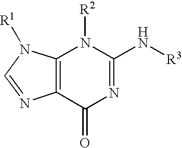

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)—X₄, —(CH₂)ₙSH, —(CH₂)ₙS—SX₅ and H, where n=1 to 10; X₄ is selected from:

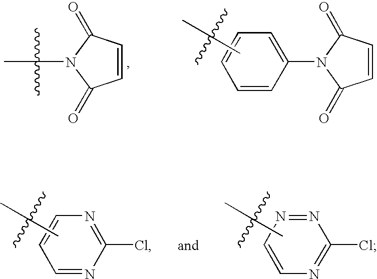

and X₅=alkyl(C₁-C₅) or —(O)₂alkyl(C₁-C₅); and

R² is selected from H and alkyl(C₁-C₅); and
R³ is selected from: H and one of the moieties A, B, C, D, and E:

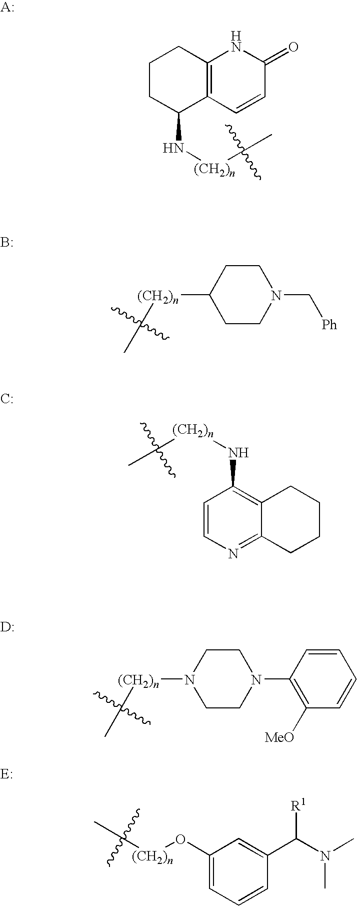

wherein E's R¹═H or Me; and n for A-E ranges from 0 to 5.

In some embodiments, a compound for use in the methods described herein is according to Formula V:

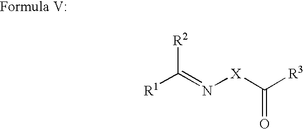

wherein X is O or NH;

wherein R¹ and R² are independently selected from H and substituted or unsubstituted, saturated or unsaturated, cyclic or linear alkyl or heteroalkyl moieties and substituted or unsubstituted aryl or heteroaryl moieties, or together R¹ and R² form a saturated or unsaturated cyclic alkyl or heteroalkyl moiety, or an aryl or heteroaryl moiety, any of which may be substituted or unsubstituted (e.g., with a fused aryl ring (which may be substituted with R⁴ and/or R⁵ moieties), or with alkyl moieties (which may be substituted with R⁴ and R⁵ moieties));

wherein R³ is selected from H, a substituted or unsubstituted alkyl, aryl, or amine moiety (e.g., substituted with R⁶ and/or R⁷ or is R⁸) or is selected from one of the moieties A, B, C, D, and E:

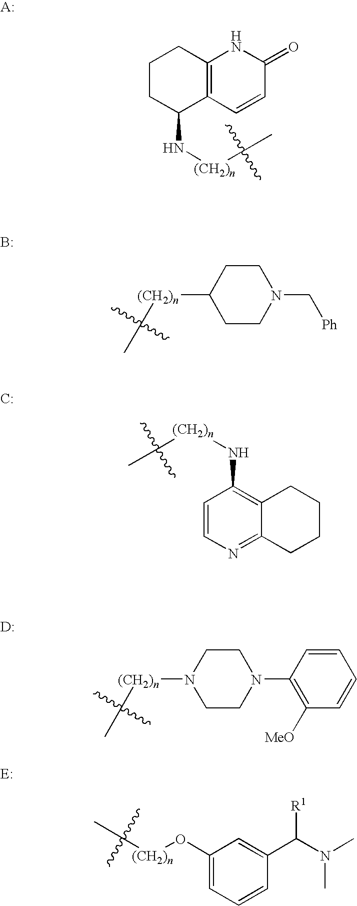

wherein E's R¹═H or Me; and n for A-E ranges from 0 to 5;

wherein R⁴ is selected from H and one of the moieties A, B, C, D, and E set forth above for R³;
wherein R⁵ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

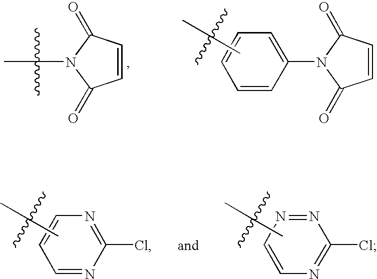

and X₅=alkyl(C₁-C₅) or —(O)₂alkyl(C₁-C₅);

wherein R⁶ and R⁷ are independently selected from H or alkyl(C₁-C₅); and
wherein R⁸ is selected from CH₃ and CH₂CH₃.

In some embodiments, a compound according to Formula V can be according to one of Formulae VA-VF, wherein Y can be O, S, or CH₂.

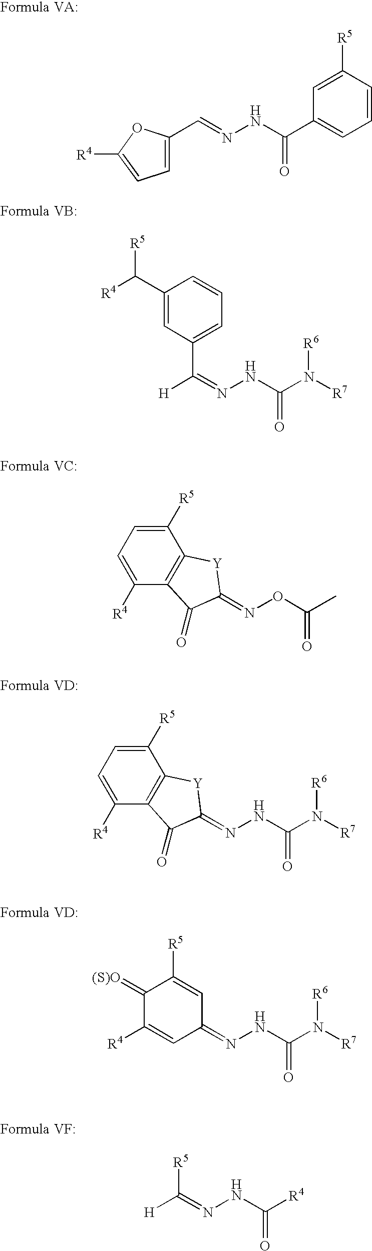

In some embodiments a compound for use in the present methods can be according to Formula VI:

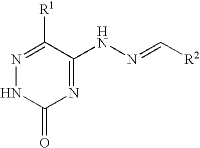

wherein R² is selected from:

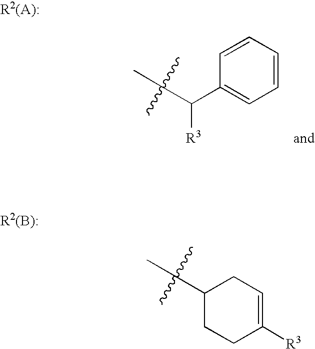

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

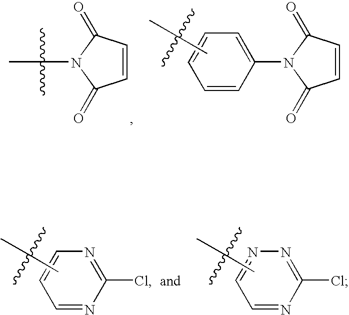

and X₅=alkyl(C₁-C₅) or —(O)₂alkyl(C₁-C₅); and

wherein R³ is selected from H and is selected from one of the moieties A, B, C, D, and E:

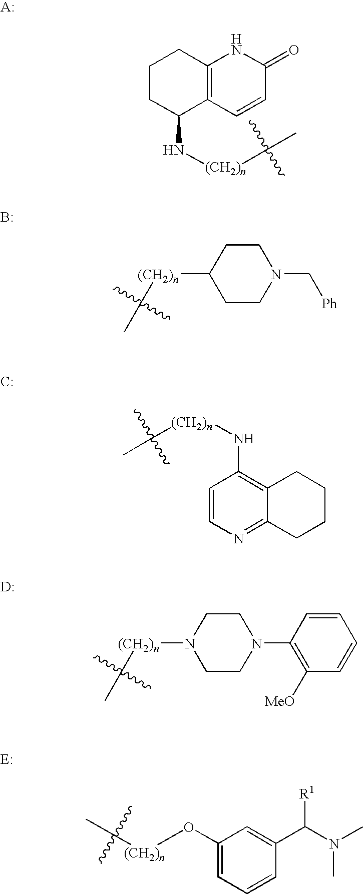

wherein E's R¹═H or Me; and n for A-E ranges from 0 to 5.

In some embodiments, a compound according to Formula VI is according to Formula VIA or VIB:

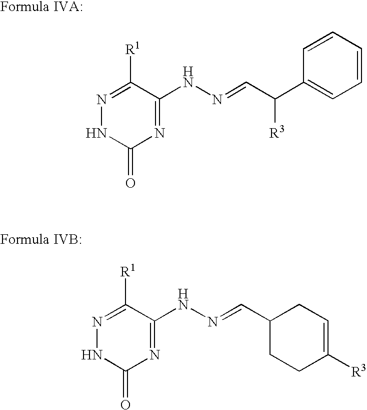

In some embodiments, a compound for use in the methods is according to Formula VII:

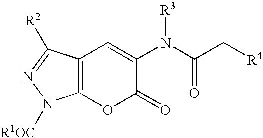

wherein R¹ and R³ are independently selected from H or alkyl(C₁-C₅);
wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

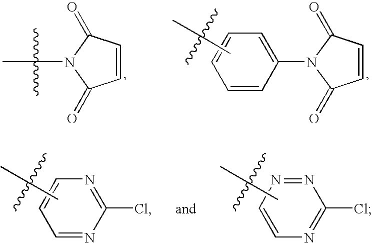

and X₅=alkyl(C₁-C₅) or —(O)₂alkyl(C₁-C₅); and

wherein R⁴ is selected from H and from one of the moieties A, B, C, D, and E:

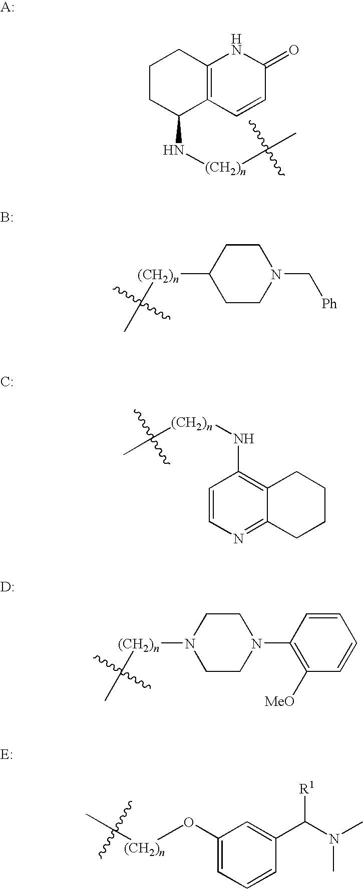

wherein E's R¹═H or Me; and n for A-E ranges from 0 to 5.

In some embodiments, a compound for use in the methods is according to Formula VIII:

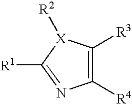

wherein R¹ is selected from H, halogen, and one of the moieties A, B, C, D, and E:

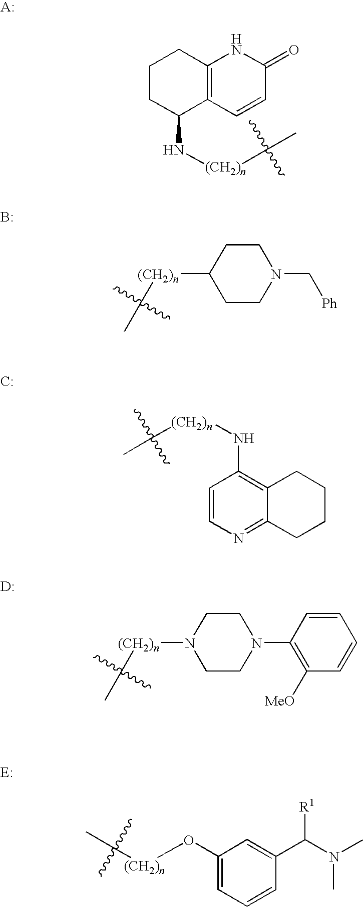

wherein E's R¹═H or Me; and n for A-E ranges from 0 to 5;

R² is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

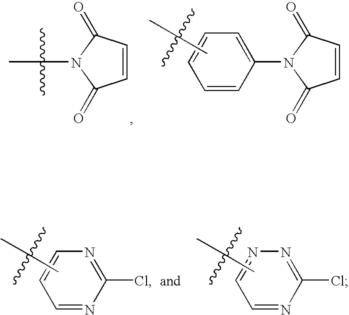

and X₅=alkyl(C₁-C₅) or —(O)₂alkyl(C₁-C₅);

wherein R³, R⁴ together form a saturated or unsaturated cyclic alkyl or cyclic heteroalkyl moiety, which may be substituted (e.g., with one or more R², R⁵, or R⁶ groups), or together form an aryl or heteroaryl moiety which may be substituted (e.g., with one or more R², R⁵, or R⁶ groups)
wherein R⁵ is selected from H or alkyl(C₁-C₅);
wherein R⁶ is selected from H and one of the moieties A, B, C, D, and E:

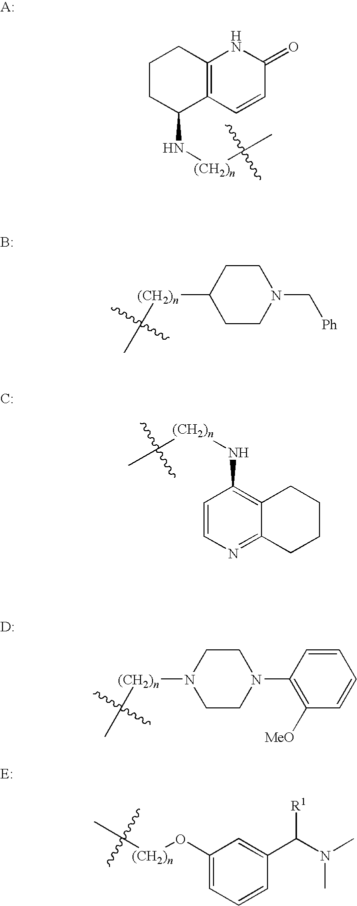

wherein E's R¹═H or Me; and n for A-E ranges from 0 to 5; and

wherein X is selected from O, S, and NH.

In some embodiments, a compound according to Formula VIII is selected from Formula VIIIA or Formula VIIIB.

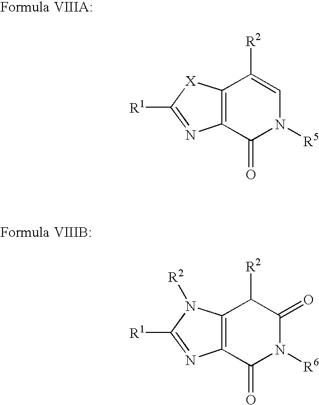

In any of the above Formulae I-VIII, X₆, if present, can be selected from:

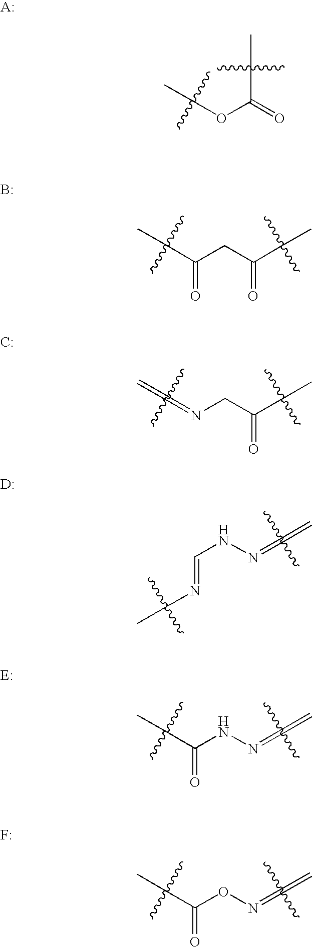

In some embodiments, a compound for use in the methods is according to Formula IX:

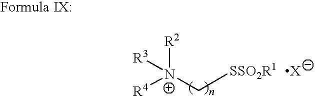

wherein R¹, R², R³ and R⁴ are independently selected from is selected from H or alkyl(C₁-C₅);

X is selected from halo and pseudohalo; and
n is an integer from 5 to 25.

In some embodiments, a compound according to Formula XI is selected from Formula IXA

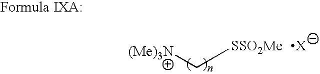

wherein X is selected from halo and pseudohalo; and

n is an integer from 5 to 25.

In some embodiments, a compound according to Formula IX is selected from Formula IXB:

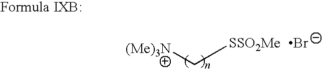

wherein n is an integer from 5 to 25.

In some embodiments, R¹, R², R³, and R⁴ are methyl. In some embodiments, X is a halo. In some embodiments, X is Br. In some embodiments, n is an integer from 7 to 20. In some embodiments, n is an integer from 17-20.

In some embodiments, a compound for use in the methods is according to Formula X:

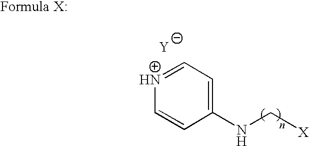

wherein X is selected from halo and pseudohalo;

Y is an salt; and
n is an integer from 5 to 15.

In some embodiments, a compound according to Formula X is selected from Formula XA

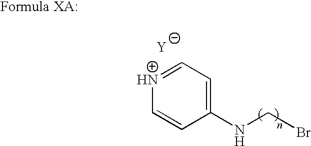

wherein Y is an salt; and

n is an integer from 5 to 15.

In some embodiments, X is a halo. In some embodiments, X is Br. In some embodiments, Y is trifluoroacetate. In some embodiments, n is an integer from 7 to 13. In some embodiments, n is 11.

In another aspect, the disclosure provides a method for killing pests comprising: providing a pesticidal composition as described herein; and applying the pesticidal composition to an area infested with pests, such that the pests can ingest or be contacted with the pesticidal composition. The pests can be selected from mosquitoes, cockroaches, lancelets, rice leaf beetles, African bollworms, beet armyworms, codling moths, diamondback moths, domestic silkworms, honey bees, oat or wheat aphids, greenbugs, melon or cotton aphids, green peach aphids, and English grain aphids.

In another embodiment, the disclosure provides a method for controlling the growth or spread of a pest population, comprising treating or contacting plants, propagation stocks, seeds, grains, foodstuffs, soils, water, industrial materials, or combinations thereof with an effective amount of a pesticidal composition described herein. Treating can include applying the composition in a manner selected from the group consisting of watering, spraying, atomizing, scattering, spreading, dry dressing, wet dressing, liquid dressing, slurry treatment of seeds, incrustation, and combinations thereof.

In another aspect, a method of controlling the mosquito-borne spread of malaria, West Nile Virus, or encephalitis is provided which includes applying an insecticidal composition described herein to an area infested with mosquitoes, such that the mosquitoes can ingest or be contacted with the insecticidal composition.

In yet another aspect, a method of controlling crop, seed, bean, foodstuff, grain, or fruit damage mediated by pests is provided and can include treating or contacting the crop, seed, bean, foodstuff, grain or fruit with a pesticidal composition described herein.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention will be apparent from the following description, from the drawings and from the claims.

### DESCRIPTION OF DRAWINGS

FIG. 1 is the SwissModel-generated multiple sequence alignments of Anopheles gambiae with mouse and electric eel acetylcholinesterases. GenBank ID of the A. gambiae acetylcholinesterases sequence: BN000066 (SEQ ID NO: 1); Protein Data Bank IDs of mouse acetylcholinesterase structures: 1J07 (SEQ ID NO: 2) and 1N5R (SEQ ID NO: 2); Protein Data Bank ID of the electric eel acetylcholinesterase structure: 1C2O (SEQ ID NO: 4). The A. gambiae-specific residues (C286 and R339) are bolded.

FIG. 2 sets forth the amber atom types and charges of acetylcholine used in the homology modeling.

FIG. 3 shows multiple sequence alignments of acetylcholinesterases of insects and mammals. The alignments were generated by CLUSTAL W (Version 1.83). C286 and R339 of Anopheles gambiae acetylcholinesterase (AChE) and the corresponding C or R residues in other species are bolded (SEQ ID NOS 5-43 are disclosed respectively in order of appearance).

FIG. 4 Multiple sequence alignments of acetylcholinesterases of the 73 species which are publicly available. The alignments were generated by CLUSTAL W (Version 1.83). C286 and R339 of Anopheles gambiae acetylcholinesterase and the corresponding C and R residues in other species are bolded (SEQ ID NOS 44-152 are disclosed respectively in order of appearance).

FIGS. 5-12 illustrate synthetic mechanisms for preparing the compositions described herein.

FIG. 13 is a block diagram of a computing system that can be used in connection with the data models and computer-implemented methods described in this document.

### DETAILED DESCRIPTION

### Definitions

As used herein, derivatives of a compound include salts, esters, enol ethers, enol esters, acetals, ketals, orthoesters, hemiacetals, hemiketals, acids, bases, solvates, or hydrates thereof. Such derivatives may be readily prepared by those of skill in this art using known methods for such derivatization. The compounds produced may be employed as insecticides and may be without substantial toxic effects to animals or humans.

Salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-l′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Solvates and hydrates are complexes of a compound with one or more solvent or water molecules, or 1 to about 100, or 1 to about 10, or one to about 2, 3 or 4, solvent or water molecules.

As used herein, “alkyl,” “alkenyl” and “alkynyl” refer to carbon chains that may be straight or branched. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propenyl).

As used herein, “cycloalkyl” refers to a saturated mono- or multi-cyclic ring system, in certain embodiments of 3 to 10 carbon atoms, in other embodiments of 3 to 6 carbon atoms. The ring systems of the cycloalkyl groups may be composed of one ring or two or more rings which may be joined together in a fused, bridged or spiro-connected fashion. Examples include, but are not limited to, cyclopropyl, cyclobutyl, cyclopentyl, and cyclohexyl.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members, where one or more, in one embodiment 1 to 4, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen.

As used herein, “carboxy” refers to a divalent radical, —C(O)O—.

As used herein, “aminocarbonyl” refers to —C(O)NH₂.

As used herein, “aminoalkyl” refers to —RNH₂, in which R is alkyl.

As used herein, “alkoxy” and “alkylthio” refer to RO— and RS—, in which R is alkyl.

As used herein, “aryloxy” and “arylthio” refer to RO— and RS—, in which R is aryl.

As used herein, “amido” refers to the divalent group —C(O)NH.

As used herein, “hydrazide” refers to the divalent group —C(O)NHNH—. Where the number of any given substituent is not specified (e.g., haloalkyl), there may be one or more substituents present. For example, “haloalkyl” may include one or more of the same or different halogens.

As used herein, the abbreviations for any protective groups or other compounds are, unless indicated otherwise, in accord with their common usage, recognized abbreviations, or the IUPAC-IUB Commission on Biochemical Nomenclature (see, (1972) Biochem. 11:942-944).

### A. Methods of Designing Inhibitors to the Ache Active Site

Provided herein are methods, including computer-based methods, for designing compounds that bind to and/or inhibit the catalytic site, peripheral site, or active site of an AChE polypeptide, particularly an invertebrate or insect AChE such as AgAChE or greenbug AChE. In some embodiments, the AChE polypeptide is a mosquito AChE, such as the house mosquito, Japanese encephalitis mosquito, or African malaria-carrying mosquito (Anopheles gambiae), including insecticide-resistant forms of the same. As used herein, the term “active site of an AChE polypeptide” includes residues that comprise the catalytic acylation site (A-site) at the base of the catalytic gorge of the polypeptide as well as residues that are understood by those having ordinary skill in the art to comprise the peripheral site (P-site) located at the entrance of the gorge; see Pang et al., Journal of Biol. Chem. 271(39):23646-23649.

The inventors have determined a refined 3D homology model of AgAChE using multiple molecular dynamics simulations. By comparing the refined 3D homology model to 3D AChE structures from other species (e.g., human AChE), and by performing multi-species AChE sequence alignments, the inventors have determined that the residues corresponding to Ag's R339 and C286 represent invertebrate—(and in some cases insect-)—specific amino acids that are located at the peripheral site of AChE and that may be involved in stabilizing active site residues. These residues are not present in the mammalian species that were compared. Thus, given the homology model described herein as well as the identification of conserved R and C residues corresponding to R339 and C286 of A. gambiae as useful invertebrate- or insect-specific residues to target, one having ordinary skill in the art would know how to use standard molecular modeling or other techniques to identify peptides, peptidomimetics, and small-molecules that would bind to or interact with one or more of the particular invertebrate's AChE's R or C residues that correspond to AgAChE's R339 and C286. In addition, one having ordinary skill in the art would be able to combine targeting such C and/or R residues with the targeting of other amino acids (such as a Trp corresponding to A. gambiae's Trp84) that are known to be within the AgAChE active site. Thus, for those insect species that have an R corresponding to Ag's R339 and/or a C corresponding to Ag's C286 as determined, e.g., by using the ClustalW alignment program, one having ordinary skill in the art would be able, given the disclosure herein, to design inhibitors of that insect's AChE that would interact with one or the other, or both, of the R or the C moiety. In some cases, a designed inhibitor would be designed to interact with both the corresponding R and C moiety. In some cases, a designed inhibitor would be designed to interact with the R moiety corresponding to Ag 339, with the C moiety corresponding to Ag C286, and with the W (tryptophan) moiety corresponding to Ag W84. In some cases, the invertebrate or insect activity would be inhibited selectively, e.g., mammalian activity would not be inhibited, with the use of one of the designed inhibitors.

In another embodiment, the inventors have determined a refined 3D homology model of greenbug AChE using multiple molecular dynamics simulations. By comparing the refined 3D homology model to 3D AChE structures from other species (e.g., human

AChE), and by performing multi-species AChE sequence alignments, the inventors have determined that the residues corresponding to greenbug's C289 represent invertebrate-(and in some cases insect-)-specific amino acids that are located at the peripheral site of AChE and that may be involved in stabilizing active site residues. This residue is not present in the mammalian species that were compared. Thus, given the homology model described herein as well as the identification of conserved C residue corresponding to

C289 of greenbug as a useful invertebrate- or insect-specific residue to target, one having ordinary skill in the art would know how to use standard molecular modeling or other techniques to identify peptides, peptidomimetics, and small-molecules that would bind to or interact with one or more of the particular invertebrate's AChE's C residue that corresponds to greenbug AChE's C289. In addition, one having ordinary skill in the art would be able to combine targeting the C residues with the targeting of other amino acids (such as a Trp corresponding to greenbug's Trp87) that are known to be within the greenbug AChE active site. Thus, for those insect species that a C corresponding to greenbug's C289 as determined, e.g., by using the ClustalW alignment program, one having ordinary skill in the art would be able, given the disclosure herein, to design inhibitors of that insect's AChE that would interact with the C moiety. In some cases, a designed inhibitor would be designed to interact with the C moiety corresponding to greenbug's C289 and with the W (tryptophan) moiety corresponding to greenbug's W87. In some cases, the invertebrate or insect activity would be inhibited selectively, e.g., mammalian activity would not be inhibited, with the use of one of the designed inhibitors.

By “molecular modeling” is meant quantitative and/or qualitative analysis of the structure and function of physical interactions based on three-dimensional structural information and interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods. See the Examples below.

Methods of designing compounds that bind specifically (e.g., with high affinity) to one or more of the residues described previously typically are also computer-based, and involve the use of a computer having a program capable of generating an atomic model. Computer programs that use X-ray crystallography data or molecular model coordinate data, such as the data that are available from the PDB, are particularly useful for designing such compounds. Programs such as RasMol, for example, can be used to generate a three dimensional model. Computer programs such as INSIGHT (Accelrys, Burlington, Mass.), Auto-Dock (Accelrys), and Discovery Studio 1.5 (Accelrys) allow for further manipulation and the ability to introduce new structures.

Compounds can be designed using, for example, computer hardware or software, or a combination of both. However, designing is preferably implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object oriented programming language to communicate with a computer system. However, the programs can be implemented in assembly or machine language, if desired. In any case, the language can be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method of the invention can also be implemented by means of a computer-readable storage medium, configured with a computer program, where the storage medium so configured causes a computer to operate in a specific and predefined manner to perform the functions described herein.

For example, a method of designing a test compound (e.g., a test inhibitor of an AChE) can involve:

(a) inputting into an input device, e.g., through a keyboard, a diskette, or a tape, data (e.g. atomic coordinates) that define the three-dimensional (3-D) structure of a first molecule or complex (e.g., an AChE polypeptide, a fragment of an AChE polypeptide, a collection of residues of an AChE polypeptide (e.g., residues making up the active site; the catalytic site, and/or the peripheral site), any of which could include a bound acetylcholine); and

(b) determining, using a processor, the 3-D structure (e.g., an atomic model) of the site on the first molecule or complex that is involved in binding to a test compound.

The method can include designing a test compound based on the determined site on the first molecule or complex that is involved in binding the test compound.

In some embodiments, a computer-assisted method of generating a test inhibitor of the acetylcholinesterase site activity of an invertebrate acetylcholinesterase (AChE) polypeptide is provided. The method uses a programmed computer comprising a processor and an input device, and can include:

(a) receiving data (e.g. atomic coordinates) comprising a docking box surrounded by one or more one residues of the active site of Botulinum as defined by the 3BOO crystal structure at a computing device;

(b) docking into the docking box a test inhibitor molecule using the processor; and

(c) determining in the computing device, based on the docking, whether the test inhibitor residue of the AChE polypeptide corresponding to Arg339 (R339) of AgAChE or Cys289 (C289) of greenbug AChE.

In some embodiments, the AChE is an insect AChE. In other embodiments, the AChE is a mosquito AChE, e.g., house mosquito, Japanese encephalitis mosquito, and African malaria mosquito. In some embodiments, the AChE is an invertebrate AChE selected from German cockroach, Florida lancelet, rice leaf beetle, African bollworm, beet armyworm, codling moth, diamondback moth, domestic silkworm, honey bee, oat or wheat aphid, the greenbug, melon or cotton aphid, green peach aphid, and English grain aphid.

By “capable of interacting” it is meant capable of forming a one or more hydrogen bonds, ionic bonds, covalent bonds, pi-pi interactions, cation-pi interactions, sulfur-aromatic interactions, or VdW interactions. In some embodiments, the test inhibitor molecule can interact with the residue corresponding to R339 of AgAChE with a minimum interaction energy of −5 to about −50 kcal/mol, e.g., −20 to −40 kcal/mol. In some cases, the residue corresponding to R339 is an arginine. In some embodiments, the test inhibitor would be capable of forming a hydrogen bond with the residue corresponding to R339.

In some embodiments, the test inhibitor is also capable of interacting with a residue of the AChE polypeptide corresponding to C286 of AgAChE. In other embodiments, the test inhibitor capable of interacting with the residue corresponding to R339 is modified and/or derivatized to be capable of interacting with a residue of the AChE polypeptide corresponding to C286 of AgAChE. In some embodiments, the residue of the AChE polypeptide corresponding to C286 is a cysteine. In some embodiments, the test inhibitor would be capable of forming a covalent bond with the residue corresponding to C286. In some embodiments, a test inhibitor that is capable of forming a covalent bond with the residue corresponding to C286 is an irreversible inhibitor.

In yet other embodiments, the test inhibitor is also capable of interacting with a residue of the AChE polypeptide corresponding to W84 of AgAChE. In other embodiments, the test inhibitor capable of interacting with the residue corresponding to R339 and/or C286 is modified and/or derivatived to be capable of interacting with a residue corresponding to W84 of AgAChE. In some embodiments, the test inhibitor is capable of interacting with residues of the AChE polypeptide corresponding to R339, C286, and W84 of AgAChE.

In some embodiments, the test inhibitor molecule can interact with the residue corresponding to C289 of greenbug AChE with a minimum interaction energy of −5 to about −50 kcal/mol, e.g., −20 to −40 kcal/mol. In some cases, the residue corresponding to C289 is a cystine. In some embodiments, the test inhibitor would be capable of forming a hydrogen bond with the residue corresponding to C289.

In some embodiments, the active site of an AChE has acetylcholine bound.

In any of the methods, the test inhibitor can be synthesized and/or derivatized so that the test inhibitor molecule is capable of interacting with a residue corresponding to

Cys286 and/or W84 of AgAChE. In any of the methods, the test inhibitor can be synthesized and/or derivatized so that the test inhibitor molecule is capable of interacting with a residue corresponding to Cys289 of greenbug AChE.

The inhibitory activity of the test inhibitor on an invertebrate or mammalian AChE polypeptide in vitro can be evaluated. The inhibitory activity of the test inhibitor on the growth of a eukaryotic (e.g., mammalian) cell can also be evaluated.

A method of generating a compound that inhibits the acetylcholinesterase site activity of the Anopheles gambiae AChE polypeptide is also provided. The method includes:

(a) providing a three-dimensional structure of the Anopheles gambiae AChE polypeptide acetylcholinesterase active site; and

(b) designing, based on the three-dimensional structure, a test compound capable of interacting with Arg339.

In some embodiments, the test compound is further capable of interacting with Cys286. In some embodiments, the test compound is derivatized to be capable of interacting with Cys286. In some embodiments, the test compound is derivatized to be capable of forming a covalent bond with Cys286. The test compound can also be capable of interacting with W84, or be derivatized to be capable of interacting with W84.

In another embodiments, a method of generating a compound that inhibits the acetylcholinesterase site activity of the greenbug AChE polypeptide is provided. The method includes:

(a) providing a three-dimensional structure of the greenbug AChE polypeptide acetylcholinesterase active site; and

(b) designing, based on the three-dimensional structure, a test compound capable of interacting with Cys289.

In some embodiments, the test compound is further capable of interacting with W87. In some embodiments, the test compound is derivatized to be capable of interacting with W87.

From the information obtained using these methods, one skilled in the art will be able to design and make inhibitory compounds (e.g., peptides, non-peptide small molecules, peptidomimetics, and aptamers (e.g., nucleic acid aptamers)) with the appropriate 3-D structure, e.g., at certain residues and that interact in certain manners (e.g., hydrogen-bonding, ion bonding, covalent bonding, pi-pi interactions, sulfur-aromatic interactions, steric interactions, and/or van der Waals interactions). For example, one of skill in the art could design inhibitory compounds that could interact with one or more of the residues corresponding to R339, C286, or Trp84 of AgAChE or C289 or Trp87 of greenbug AChE. It should be noted that although the original AChE polypeptide 3-D structure may be taken from one species (e.g., AgAChE or greenbug AChE), one of skill in art could, by standard methods, e.g., homology alignments (i.e., ClustalW (1.83)) or molecular modeling, establish the corresponding residues of interest in other species.

Moreover, if computer-usable 3-D data (e.g., x-ray crystallographic data) for a candidate compound are available, one or more of the following computer-based steps can be performed in conjunction with computer-based steps described above:

(c) inputting into an input device, e.g., through a keyboard, a diskette, or a tape, data (e.g. atomic coordinates) that define the three-dimensional (3-D) structure of a candidate compound;

(d) determining, using a processor, the 3-D structure (e.g., an atomic model) of the candidate compound;

(e) determining, using the processor, whether the candidate compound binds to or interacts with the residues of interest in the first molecule or complex; and

(f) identifying the candidate compound as a compound that inhibits the site.

The method can involve an additional step of outputting to an output device a model of the 3-D structure of the compound. In addition, the 3-D data of candidate compounds can be compared to a computer database of, for example, 3-D structures stored in a data storage system.

Candidate compounds identified as described above can then be tested in standard cellular or cell-free enzymatic or enzymatic inhibition assays familiar to those skilled in the art. Inhibitory activity can be compared with inhibition to one or more mammalian (e.g., human, cat, dog, mouse, rat, monkey, horse, cow) AChEs.

The 3-D structure of molecules can be determined from data obtained by a variety of methodologies. These methodologies include: (a) x-ray crystallography; (b) nuclear magnetic resonance (NMR) spectroscopy; (c) molecular modeling methods, e.g., homology modeling techniques, threading algorithms, and in particular the refined homology modeling methods described below in the Examples.

Any available method can be used to construct a 3-D model of an AChE region of interest, such as the active site, peripheral site, or catalytic site, from the x-ray crystallographic, molecular modeling, and/or NMR data using a computer as described above. Such a model can be constructed from analytical data points inputted into the computer by an input device and by means of a processor using known software packages, e.g., CATALYST (Accelrys), INSIGHT (Accelrys) and CeriusII, HKL, MOSFILM, XDS, CCP4, SHARP, PHASES, HEAVY, XPLOR, TNT, NMRCOMPASS, NMRPIPE, DIANA, NMRDRAW, FELIX, VNMR, MADIGRAS, QUANTA, BUSTER, SOLVE, O, FRODO, or CHAIN. The model constructed from these data can be visualized via an output device of a computer, using available systems, e.g., Silicon Graphics, Evans and Sutherland, SUN, Hewlett Packard, Apple Macintosh, DEC, IBM, or Compaq.

Once the 3-D structure of a compound that binds to or interacts with one or more residues of an AChE that correspond to R339, C286, or Trp84 of AgAChE has been established using any of the above methods, a compound that has substantially the same 3-D structure (or contains a domain that has substantially the same structure) as the identified compound can be made. In this context, “has substantially the same 3-D structure” means that the compound possesses a hydrogen bonding and hydrophobic character that is similar to the identified compound. In some cases, a compound having substantially the same 3-D structure as the identified compound can include a heterocyclic ring system and regions displaying hydrophobic character in close proximity to a hydrogen bonding region, although the hydrophobic regions can contain some hydrogen bonding character. Compounds of this class would include, without limitation, substituents able to impart steric bulk in a region of space that would otherwise encapsulate the manganese and carbonate-coordinated phosphate backbone characteristic of an identified compound.

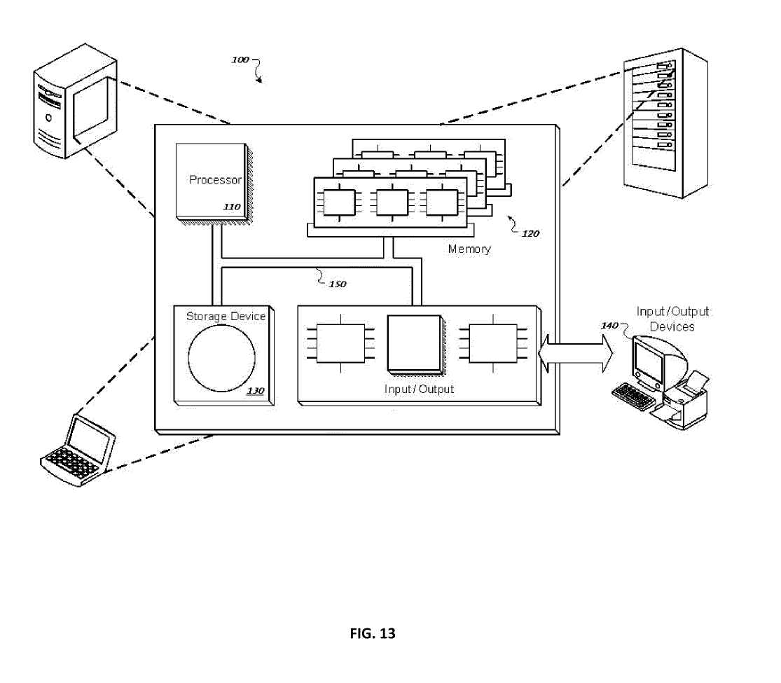

FIG. 13 is a schematic diagram of a computer system 100. The system 100 can be used for the operations described in association with any of the computer-implement methods described previously, according to one embodiment. The system 100 is intended to include various forms of digital computers, such as laptops, desktops, workstations, personal digital assistants, servers, blade servers, mainframes, and other appropriate computers. The system 100 can also include mobile devices, such as personal digital assistants, cellular telephones, smartphones, and other similar computing devices.

Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The system 100 includes a processor 110, a memory 120, a storage device 130, and an input/output device 140. Each of the components 110, 120, 130, and 140 are interconnected using a system bus 150. The processor 110 is capable of processing instructions for execution within the system 100. The processor may be designed using any of a number of architectures. For example, the processor 110 may be a CISC (Complex Instruction Set Computers) processor, a RISC (Reduced Instruction Set Computer) processor, or a MISC (Minimal Instruction Set Computer) processor.

In one embodiment, the processor 110 is a single-threaded processor. In another embodiment, the processor 110 is a multi-threaded processor. The processor 110 is capable of processing instructions stored in the memory 120 or on the storage device 130 to display graphical information for a user interface on the input/output device 140. The memory 120 stores information within the system 100. In one embodiment, the memory 120 is a computer-readable medium. In one embodiment, the memory 120 is a volatile memory unit. In another embodiment, the memory 120 is a non-volatile memory unit.

The storage device 130 is capable of providing mass storage for the system 100. In one embodiment, the storage device 130 is a computer-readable medium. In various different embodiments, the storage device 130 may be a floppy disk device, a hard disk device, an optical disk device, or a tape device.

The input/output device 140 provides input/output operations for the system 100. In one embodiment, the input/output device 140 includes a keyboard and/or pointing device. In another embodiment, the input/output device 140 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described embodiments by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

With the above described 3-D structural data in hand and knowing the chemical structure (e.g., amino acid sequence in the case of a protein) of the region of interest, those of skill in the art would know how to make compounds with the above-described properties. Moreover, one having ordinary skill in the art would know how to derivatize such compounds. Such methods include chemical synthetic methods and, in the case of proteins, recombinant methods.

While not essential, computer-based methods can be used to design the compounds of the invention. Appropriate computer programs include: InsightII (Accelrys), CATALYST (Accelrys), LUDI (Accelrys., San Diego, Calif.), Aladdin (Daylight Chemical Information Systems, Irvine, Calif.); and LEGEND [Nishibata et al. (1985) J. Med. Chem. 36(20):2921-2928], as well as the methods described in the Examples below and the references cited therein.

Compounds of the invention can be modified by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptide compounds can be covalently or noncovalently coupled to pharmaceutically acceptable “carrier” proteins prior to administration.

Also of interest are peptidomimetic compounds. Peptidomimetic compounds are synthetic compounds having a three-dimensional conformation (i.e., a “peptide motif”) that is substantially the same as the three-dimensional conformation of a selected peptide. Peptidomimetic compounds can have additional characteristics that enhance their in vivo utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

Small-molecule compounds that can bind to and/or interact with a residue corresponding to R339 of AgAChE, and/or the residue corresponding to C286 of AgAChE, and/or the residue corresponding to W84 of AgAChE, including in particular those that are trifunctional, e.g., are designed to bind to the residues corresponding to R339, C286, and W84 of AgAChE, are of particular interest. In addition, small-molecule compounds that can bind to and/or interact with a residue corresponding to C289 of greenbug, and/or the residue corresponding to W87 of greenbug AChE, including in particular those that are difunctional, e.g., are designed to bind to the residues corresponding to C289 and W87 of greenbug AChE, are of interest. Additional information on particular classes of small molecules is provided below, as well as synthetic methodologies for preparation of such molecules.

### B. Compounds and Compositions

The compounds provided herein may specifically inhibit invertebrate (e.g., insect) acetylcholinesterase activity as compared to mammalian acetylcholinesterase activity, and thus may be useful as safe, non-toxic pesticides and insecticides. For example, the compounds provided herein may inhibit the AChE of the house mosquito, Japanese encaphilitis mosquito, and/or African malaria mosquito, and thus may be useful to specifically target populations of mosquitoes, e.g., to prevent the spread of malaria or other mosquito-borne diseases such as malaria, West Nile virus, encephalitis, etc. In other cases, the compounds may inhibit the AChE of other invertebrate pest species, including cockroaches, beetles, bollworms, moths, aphids, and greenbugs, and thus can be used as pesticides that are useful in controlling pest populations e.g., on farms and in food storage and transportation facilities.

Use of any of the compounds provided herein, or their acceptable salts or derivatives, as pesticides or insecticides, e.g., to kill insect populations, to prevent or minimize crop damage, or to prevent or minimize the spread of insect-borne diseases, is also contemplated.

Compounds for use in the compositions and methods provided herein, or acceptable salts or derivatives thereof, can be according to Formula I:

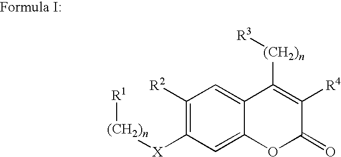

wherein n=0 to 3;
wherein R¹ is selected from:

a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

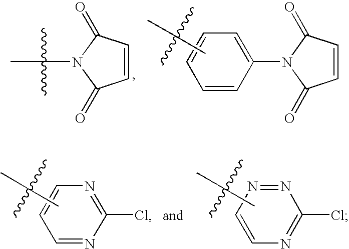

and X₅=alkyl(C₁-C₅) or —(O)₂alkyl(C₁-C₅); and

b) H and one of the moieties A, B, C, D, and E:

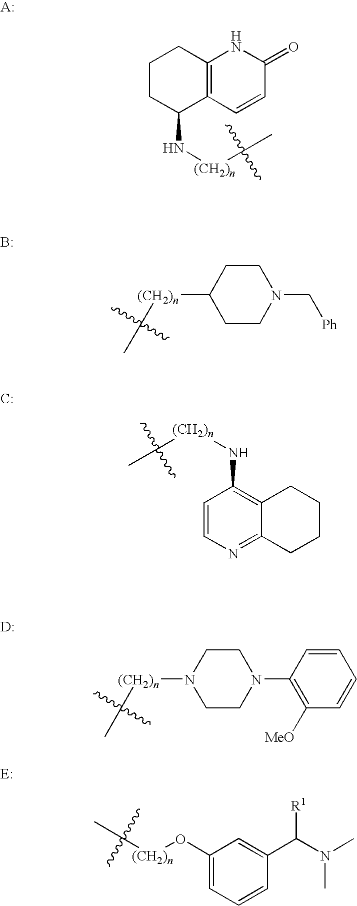

wherein E's R¹═H or Me; and n for A-E ranges from 0 to 5;

wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10, X₄ is as above, and X₅=alkyl(C₁-C₅) or —(O)₂alkyl(C₁-C₅);
wherein R³ is selected from H, CH₃, and

a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅, where n=1 to 10, X⁴ is as above, and X₅=alkyl(C₁-C₅) or —(O)₂alkyl(C₁-C₅); and

b) one of the moieties A, B, C, D, and E:

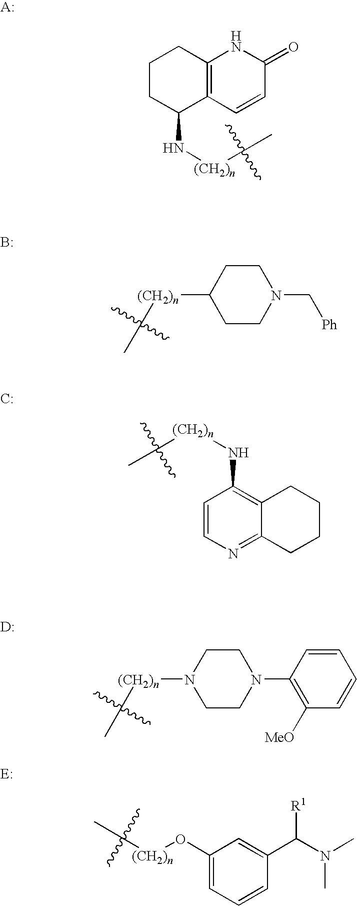

wherein E's R¹═H or Me; and n for A-E ranges from 0 to 5;

wherein R⁴ is selected from:

H and one of the moieties A, B, C, D, and E:

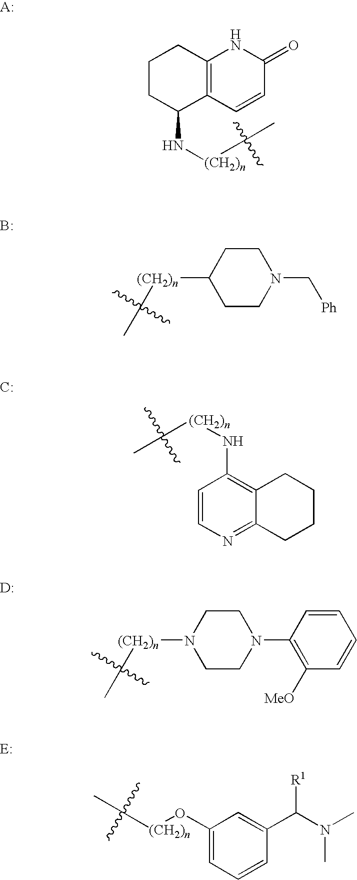

wherein E's R¹═H or Me; and n for A-E ranges from 0 to 5;

wherein X═O or NR⁵, wherein R⁵ is selected from H or C₁-C₅ alkyl.

In some embodiments, a compound according to Formula I is a compound according to Formula IA:

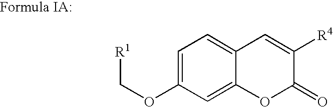

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CHASH, —(CH₂)ₙS—SX₅ and H, where n=1 to 10; X₄ is selected from:

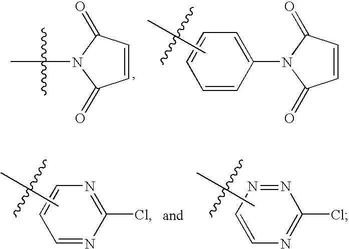

and X₅=alkyl(C₁-C₅) or —(O)₂alkyl(C₁-C₅); and

wherein R⁴ is selected from: H and one of the moieties A, B, C, D, and E:

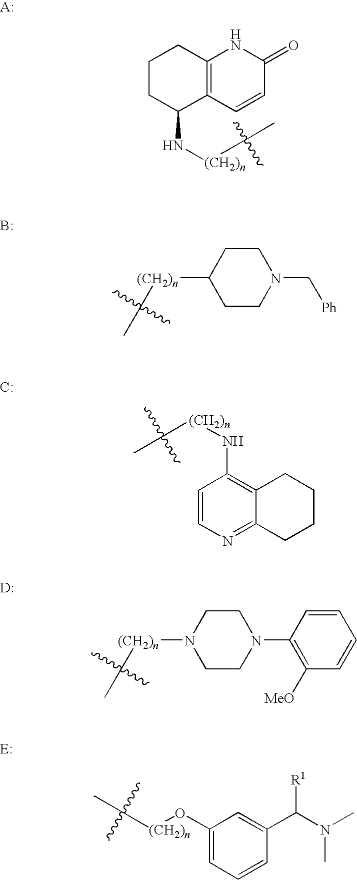

wherein E's R¹═H or Me; and n for A-E ranges from 0 to 5.

In some embodiments, a compound according to Formula I is according to Formula IB:

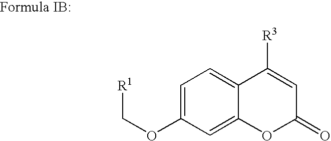

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

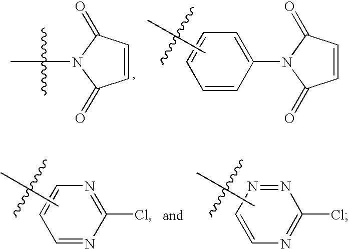

and X₅=alkyl(C₁-C₅) or —(O)₂alkyl(C₁-C₅); and

wherein R³ is selected from H and one of the moieties A, B, C, D, and E:

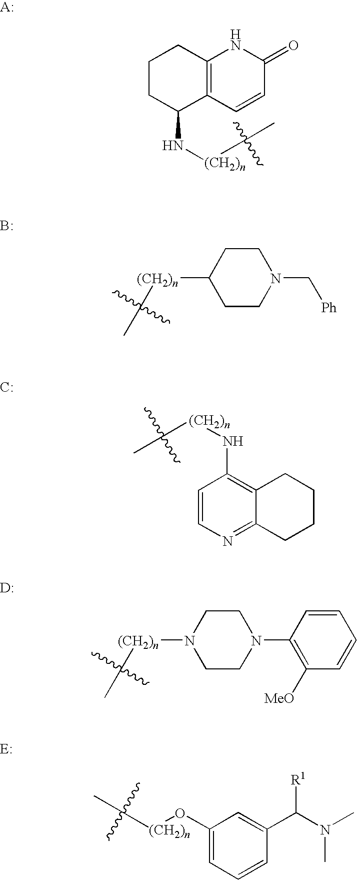

wherein E's R¹═H or Me; and n for A-E ranges from 0 to 5.

In some embodiments, a compound according to Formula I is according to Formula IC:

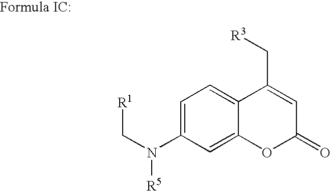

wherein R¹ is selected from H and one of the moieties A, B, C, D, and E:

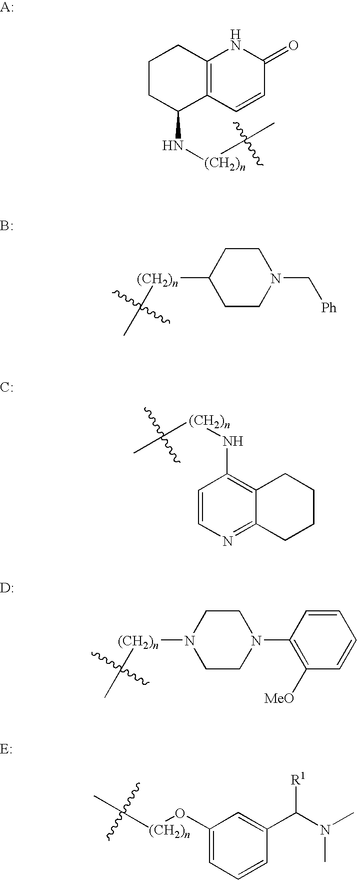

wherein E's R¹═H or Me; and n for A-E ranges from 0 to 5;

wherein R³ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

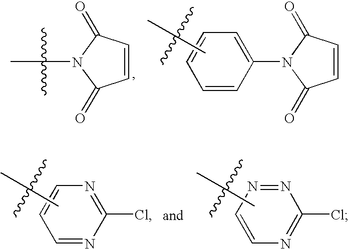

and X₅=alkyl(C₁-C₅) or —(O)₂alkyl(C₁-C₅); and

R⁵ is as above in Formula I.

In some embodiments, a compound according to Formula I is according to Formula ID:

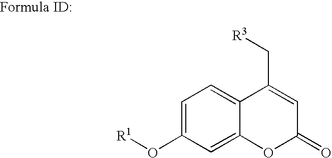

wherein R¹ is selected from selected from H and one of the moieties A, B, C, D, and E:

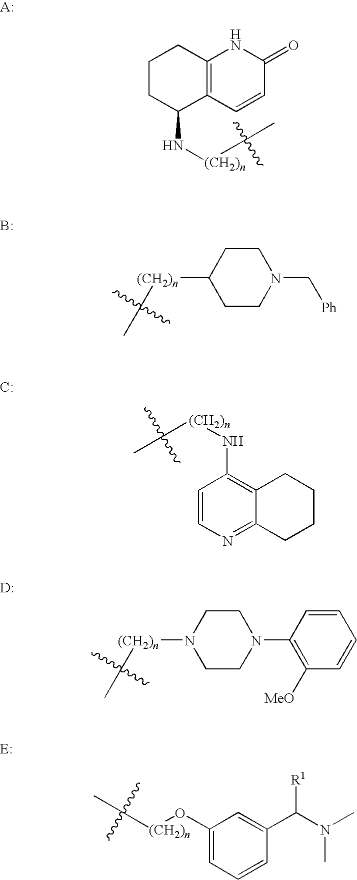

wherein E's R¹═H or Me; and n for A-E ranges from 0 to 5;

wherein R³ selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

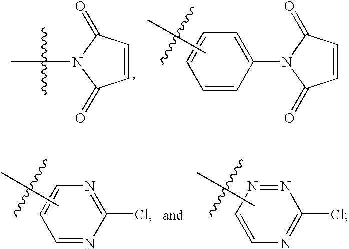

and X₅=alkyl(C₁-C₅) or —(O)₂alkyl(C₁-C₅).

In some embodiments, a compound according to Formula I is according to Formula IE:

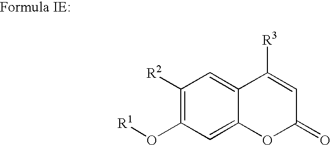

wherein R³ is H or CH₃;
wherein R¹ is selected from H and one of the moieties A, B, C, D, and E:

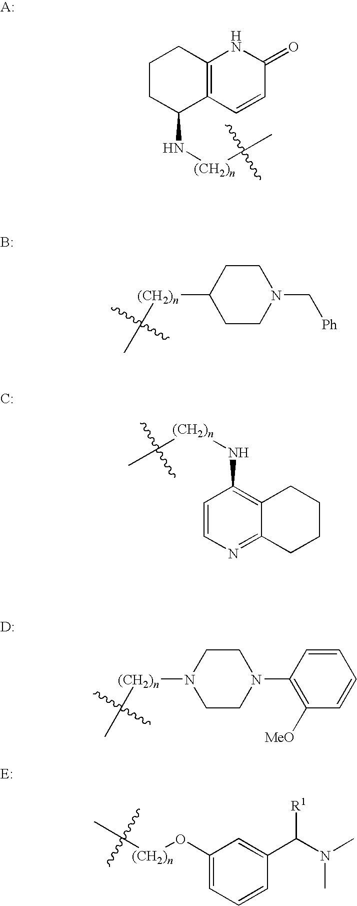

wherein E's R¹═H or Me; and n for A-E ranges from 0 to 5; and

wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

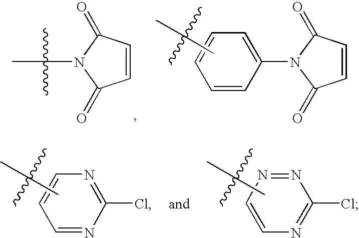

and X₅=alkyl(C₁-C₅) or —(O)₂alkyl(C₁-C₅).
In some embodiments, a compound for use in the present methods is according to Formula II:

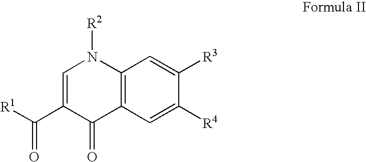

wherein R¹ is H, OMe, OEt, NH₂, alkyl(C₁-C₅), or is selected from:

### A, B, C, D, and E:

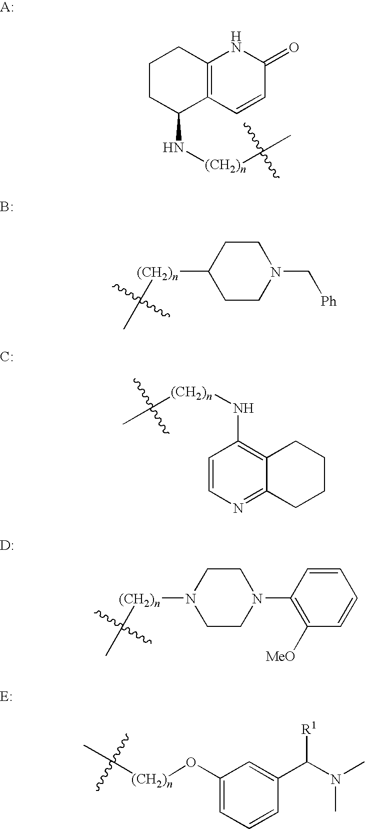

wherein E's R¹═H or Me; and n for A-E ranges from 0 to 5;
wherein R² and R³ are independently selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

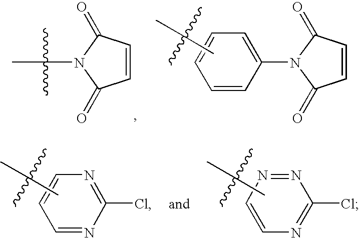

and X₅=alkyl(C₁-C₅) or —(O)₂alkyl(C₁-C₅);

wherein R⁴ is selected from: H and one of the moieties A, B, C, D, and E:

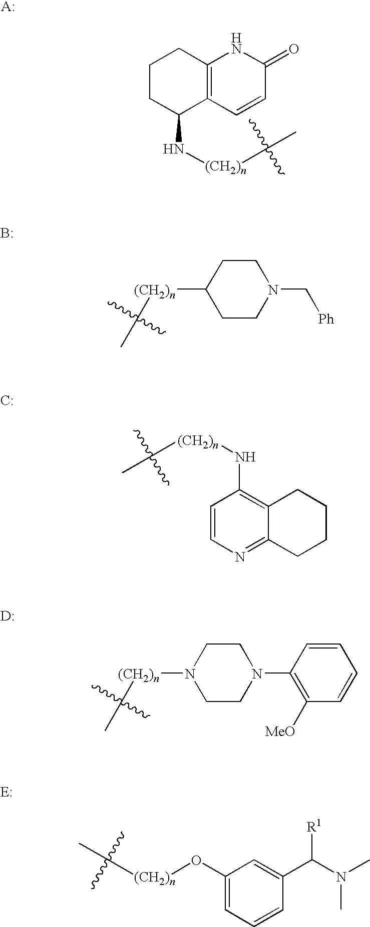

wherein E's R¹═H or Me; and n for A-E ranges from 0 to 5.

In some embodiments, a compound according to Formula II is according to Formula IIA or Formula IIB, below:

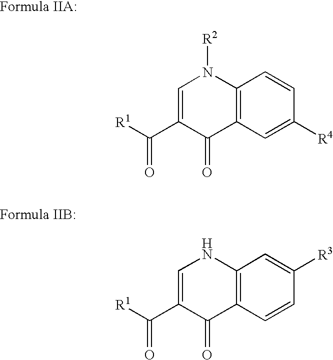

In some embodiments, a compound for use in the methods described herein is according to Formula IIIA or Formula IIIB:

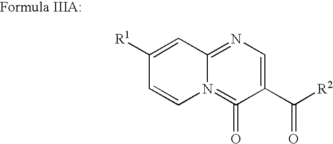

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

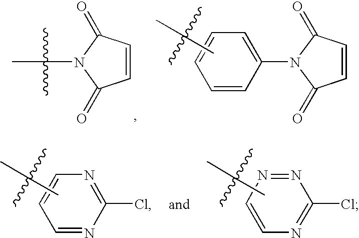

and X₅=alkyl(C₁-C₅) or —(O)₂alkyl(C₁-C₅); and

wherein R² is selected from: H and one of the moieties A, B, C, D, and E:

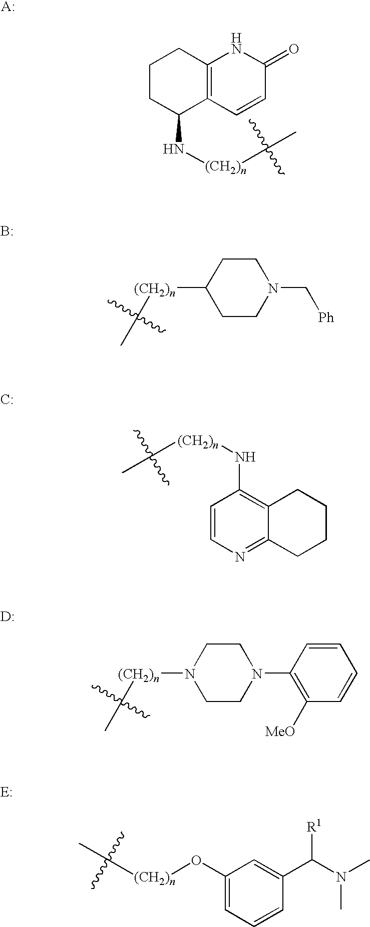

wherein E's R¹═H or Me; and n for A-E ranges from 0 to 5.

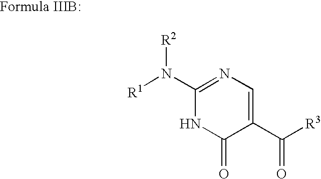

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

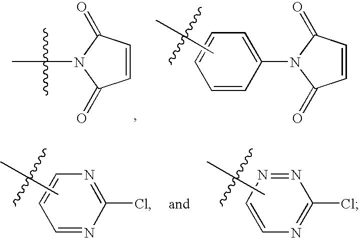

and X₅=alkyl(C₁-C₅) or —(O)₂alkyl(C₁-C₅); and

R² is selected from H and alkyl(C₁-C₅); and
R³ is selected from: H and one of the moieties A, B, C, D, and E:

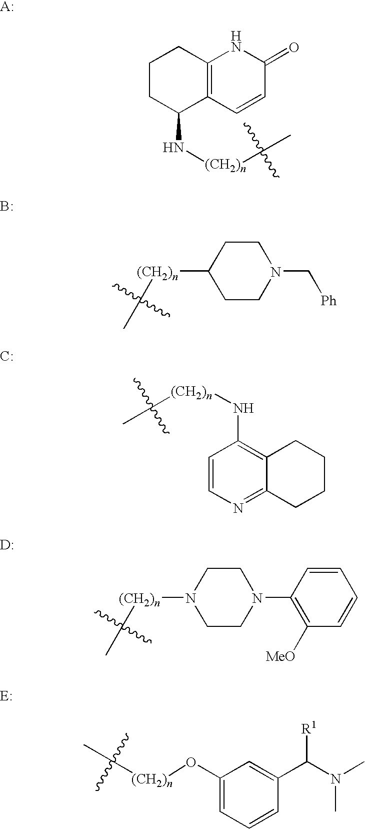

wherein E's R¹═H or Me; and n for A-E ranges from 0 to 5.

In some embodiments, a compound for use in the invention is according to Formula IV:

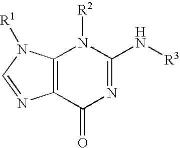

wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

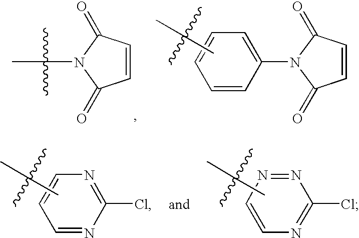

and X₅=alkyl(C₁-C₅) or —(O)₂alkyl(C₁-C₅); and

R² is selected from H and alkyl(C₁-C₅); and
R³ is selected from: H and one of the moieties A, B, C, D, and E:

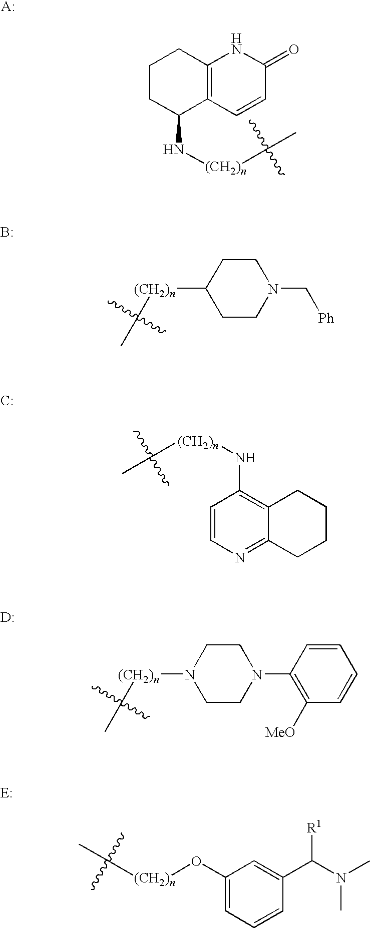

wherein E's R¹═H or Me; and n for A-E ranges from 0 to 5.

In some embodiments, a compound for use in the methods described herein is according to Formula V:

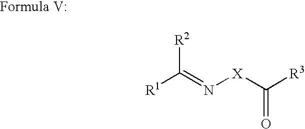

wherein X is O or NH;

wherein R¹ and R² are independently selected from H and substituted or unsubstituted, saturated or unsaturated, cyclic or linear alkyl or heteroalkyl moieties and substituted or unsubstituted aryl or heteroaryl moieties, or together R¹ and R² form a saturated or unsaturated cyclic alkyl or heteroalkyl moiety, or an aryl or heteroaryl moiety, any of which may be substituted or unsubstituted (e.g., with a fused aryl ring (which may be substituted with R⁴ and/or R⁵ moieties), or with alkyl moieties (which may be substituted with R⁴ and R⁵ moieties));

wherein R³ is selected from H, a substituted or unsubstituted alkyl, aryl, or amine moiety (e.g., substituted with R⁶ and/or R⁷ or is R⁸) or is selected from one of the moieties A, B, C, D, and E:

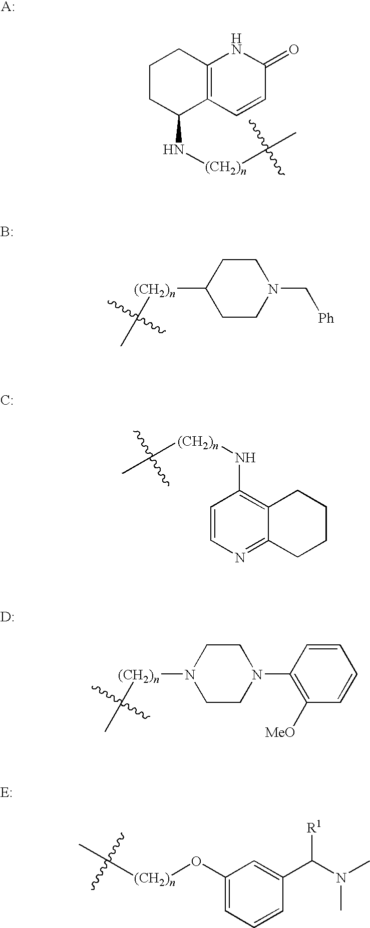

wherein E's R¹═H or Me; and n for A-E ranges from 0 to 5;

wherein R⁴ is selected from H and one of the moieties A, B, C, D, and E set forth above for R³;
wherein R⁵ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

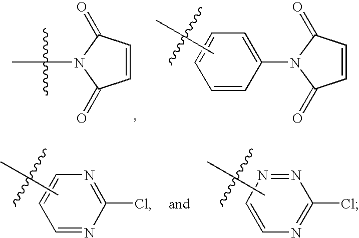

and X₅=alkyl(C₁-C₅) or —(O)₂alkyl(C₁-C₅);

wherein R⁶ and R⁷ are independently selected from H or alkyl(C₁-C₅); and
wherein R⁸ is selected from CH₃ and CH₂CH₃.

In some embodiments, a compound according to Formula V can be according to one of Formulae VA-VF, wherein Y can be O, S, or CH₂.

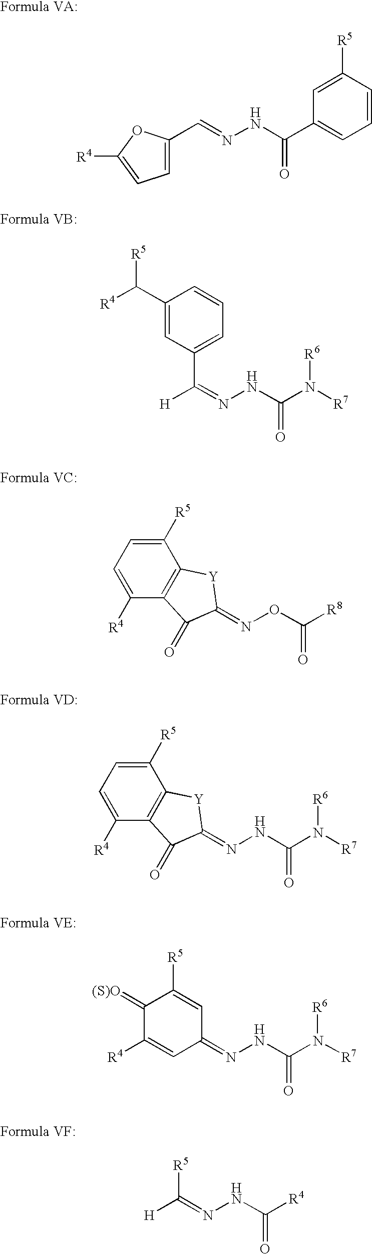

In some embodiments a compound for use in the present methods can be according to Formula VI:

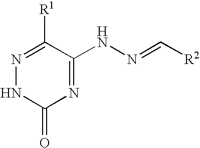

wherein R² is selected from:

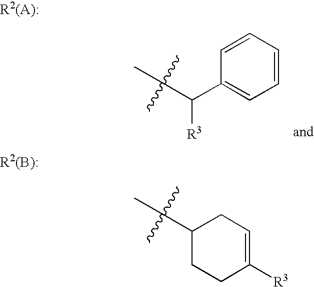

wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

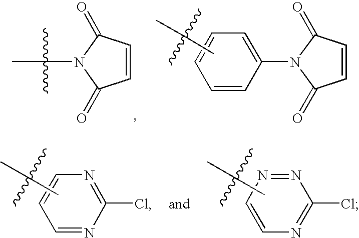

and X₅=alkyl(C₁-C₅) or —(O)₂alkyl(C₁-C₅); and

wherein R³ is selected from H and is selected from one of the moieties A, B, C, D, and E:

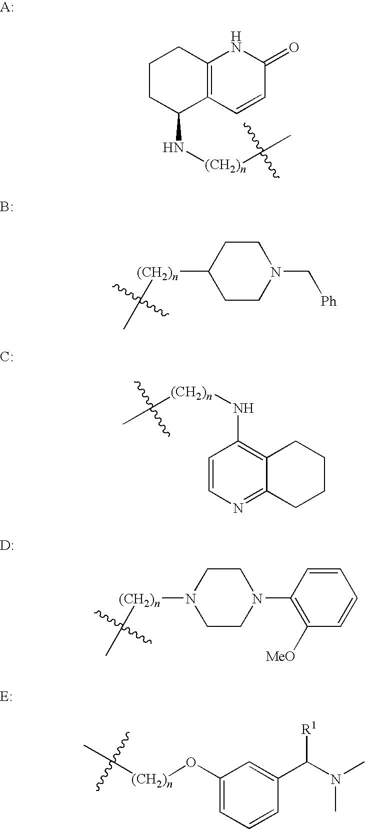

wherein E's R¹═H or Me; and n for A-E ranges from 0 to 5.

In some embodiments, a compound according to Formula VI is according to Formula VIA or VIB:

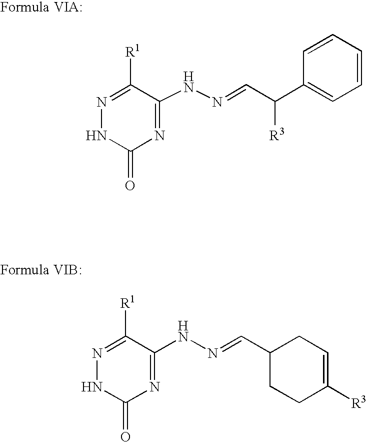

In some embodiments, a compound for use in the methods is according to Formula VII:

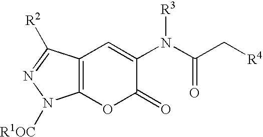

wherein R¹ and R³ are independently selected from H or alkyl(C₁-C₅); wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

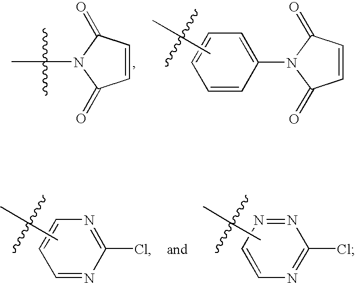

and X₅=alkyl(C₁-C₅) or —(O)₂alkyl(C₁-C₅); and

wherein R⁴ is selected from H and from one of the moieties A, B, C, D, and E:

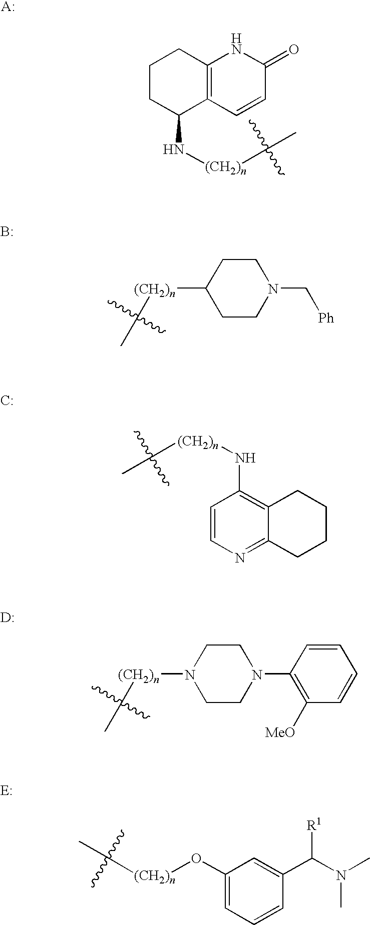

wherein E's R¹═H or Me; and n for A-E ranges from 0 to 5.

In some embodiments, a compound for use in the methods is according to Formula VIII:

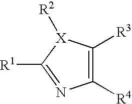

wherein R¹ is selected from H, halogen, and one of the moieties A, B, C, D, and E:

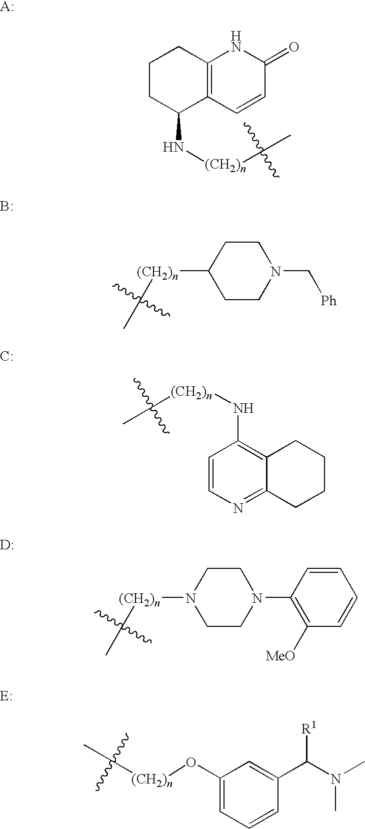

wherein E's R¹═H or Me; and n for A-E ranges from 0 to 5;

R² is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from:

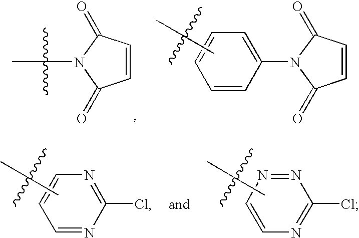

and X₅=alkyl(C₁-C₅) or —(O)₂alkyl(C₁-C₅);
wherein R³, R⁴ together form a saturated or unsaturated cyclic alkyl or cyclic heteroalkyl moiety, which may be substituted (e.g., with one or more R², R⁵, or R⁶ groups), or together form an aryl or heteroaryl moiety which may be substituted (e.g., with one or more R², R⁵, or R⁶ groups)
wherein R⁵ is selected from H or alkyl(C₁-C₅);
wherein R⁶ is selected from H and one of the moieties A, B, C, D, and E:

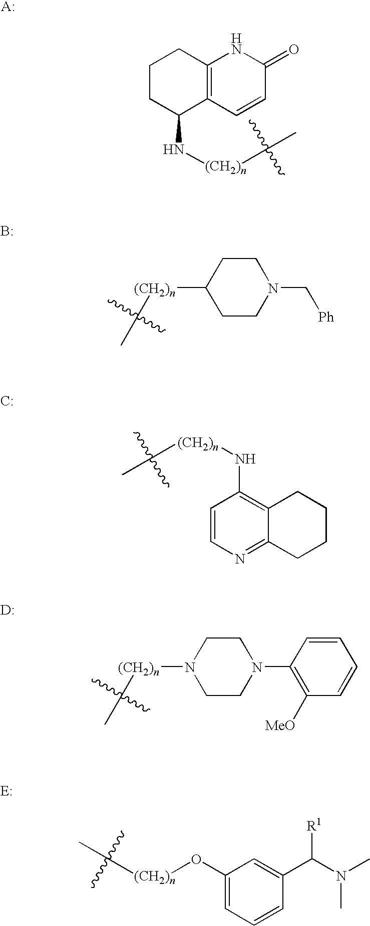

wherein E's R¹═H or Me; and n for A-E ranges from 0 to 5; and

wherein X is selected from O, S, and NH.

In some embodiments, a compound according to Formula VIII is selected from Formula VIIIA or Formula VIIIB.

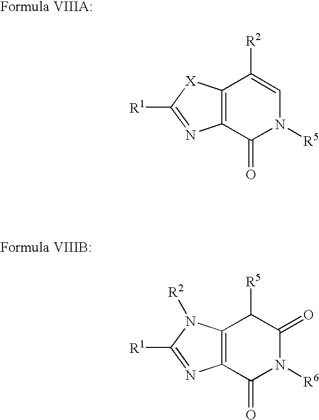

In any of the above Formulae I-VIII, X₆, if present, can be selected from:

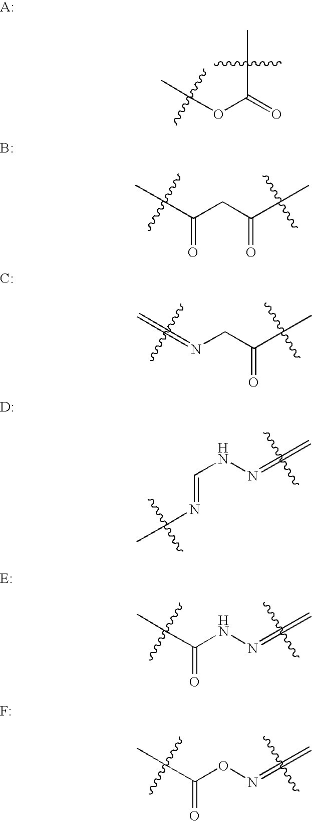

In some embodiments, a compound for use in the methods is according to Formula IX:

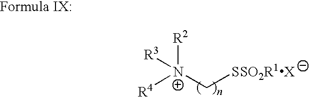

wherein R¹, R², R³ and R⁴ are independently selected from is selected from H or alkyl(C₁-C₅);

X is selected from halo and pseudohalo; and
n is an integer from 5 to 25.

In some embodiments, a compound according to Formula XI is selected from Formula IXA

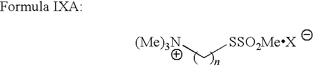

wherein X is selected from halo and pseudohalo; and

n is an integer from 5 to 25.
In some embodiments, a compound according to Formula IX is selected from Formula IXB:

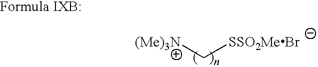

wherein n is an integer from 5 to 25.

In some embodiments, R¹, R², R³, and R⁴ are methyl. In some embodiments, X is a halo. In some embodiments, X is Br. In some embodiments, n is an integer from 7 to 20. In some embodiments, n is an integer from 17-20.

In some embodiments, a compound for use in the methods is according to Formula X:

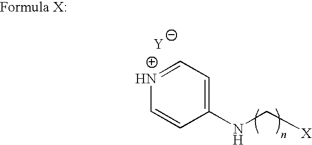

wherein X is selected from halo and pseudohalo;

Y is an salt; and
n is an integer from 5 to 15.

In some embodiments, a compound according to Formula X is selected from Formula XA

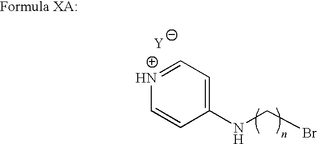

wherein Y is an salt; and

n is an integer from 5 to 15.

In some embodiments, X is a halo. In some embodiments, X is Br. In some embodiments, Y is trifluoroacetate. In some embodiments, n is an integer from 7 to 13. In some embodiments, n is 11.

### C. Preparation of the Compounds

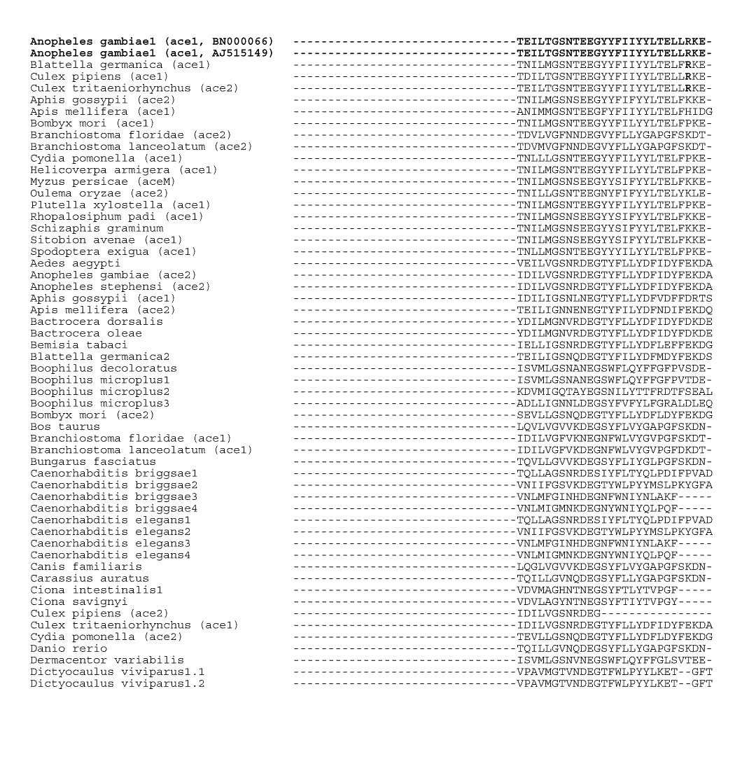

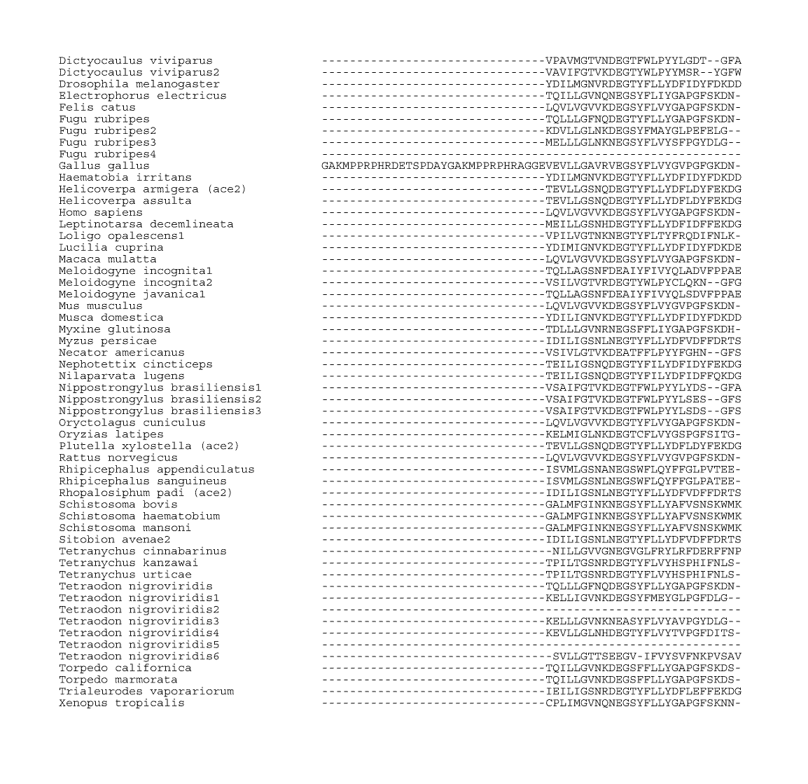

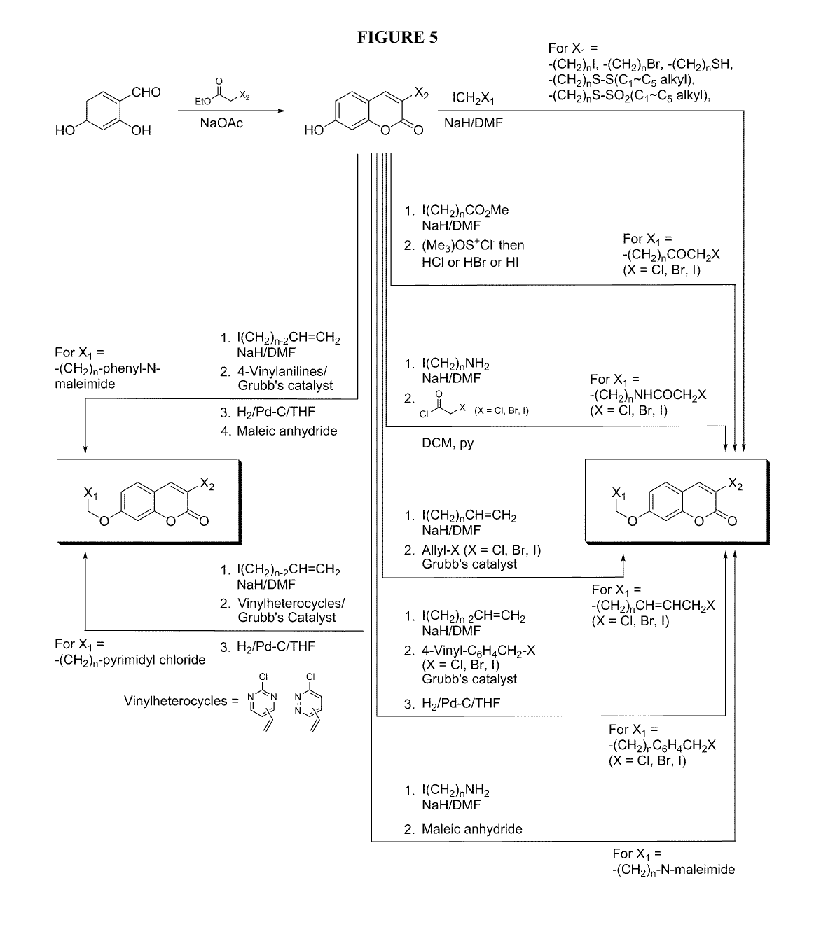

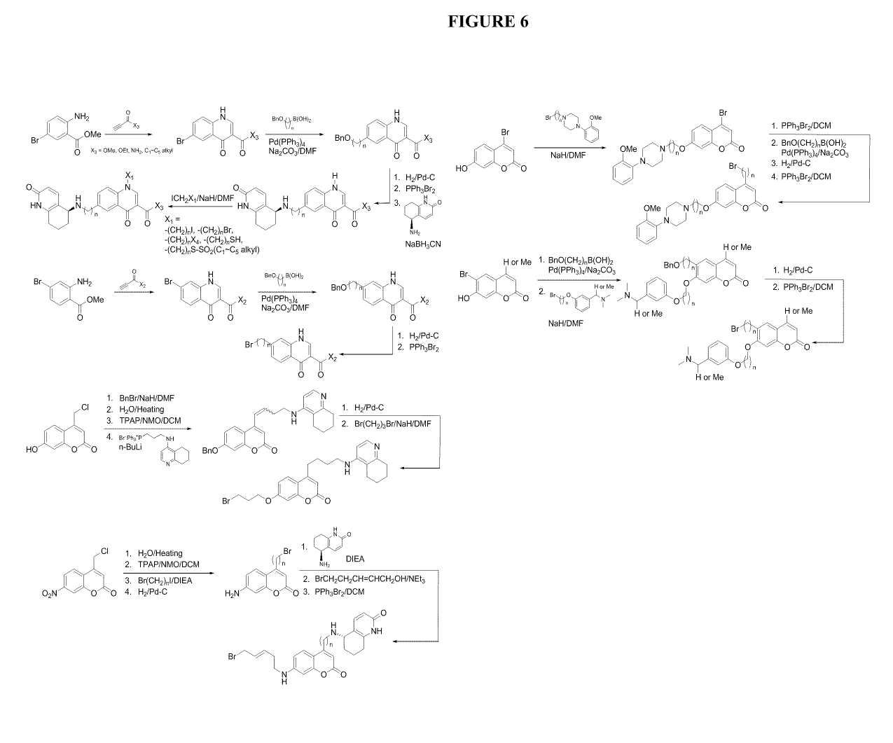

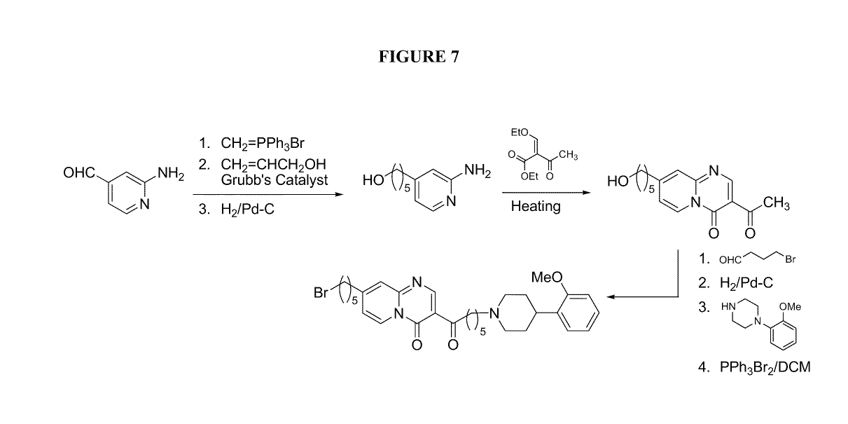

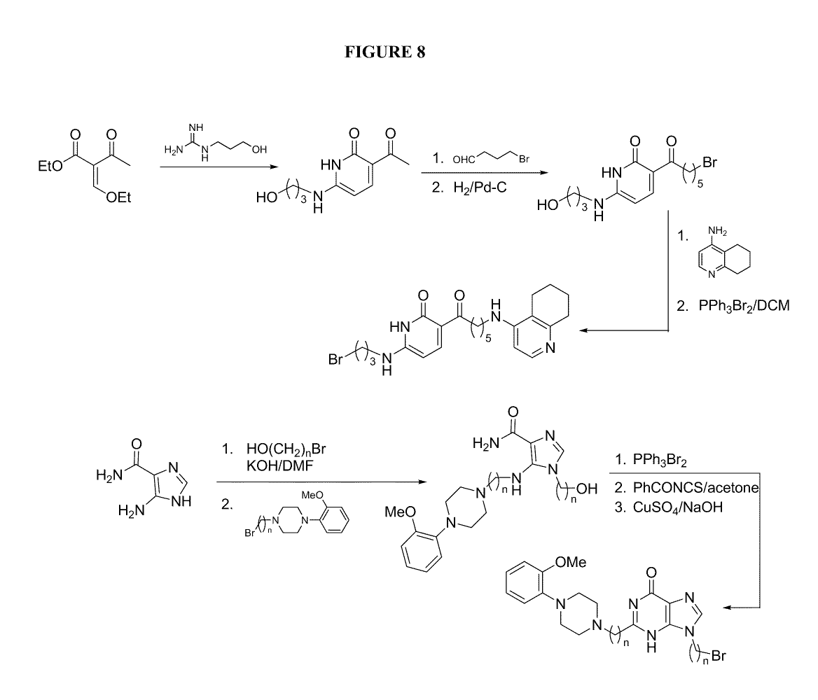

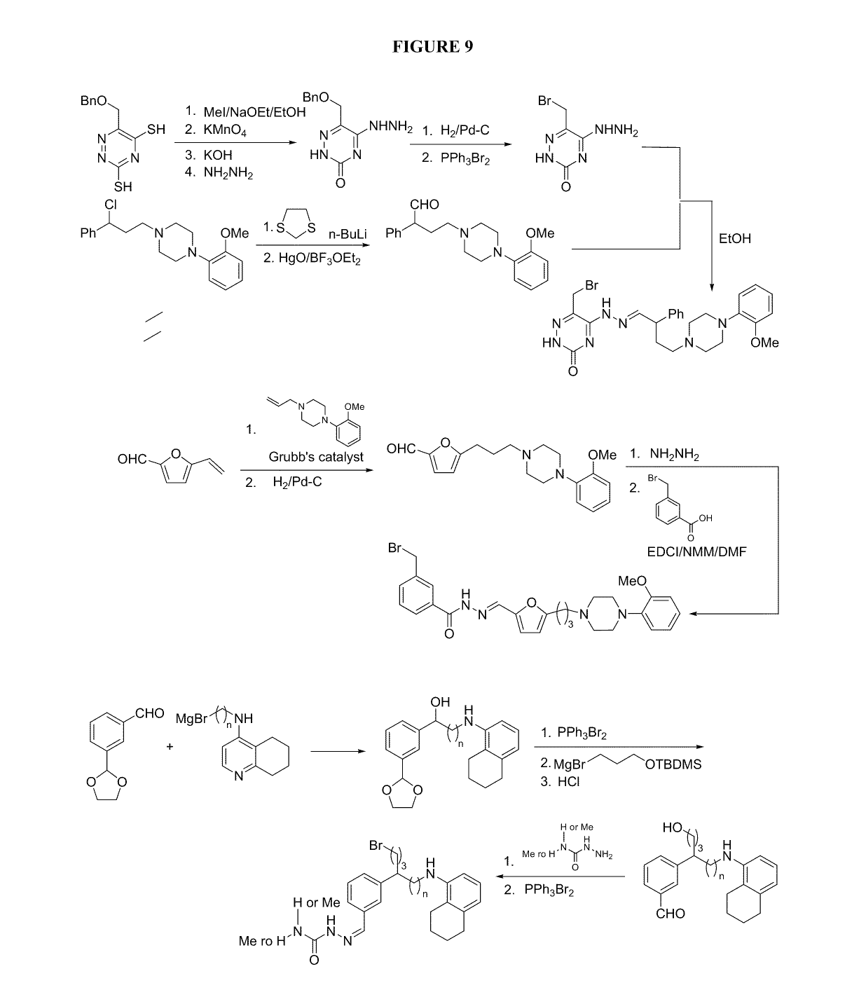

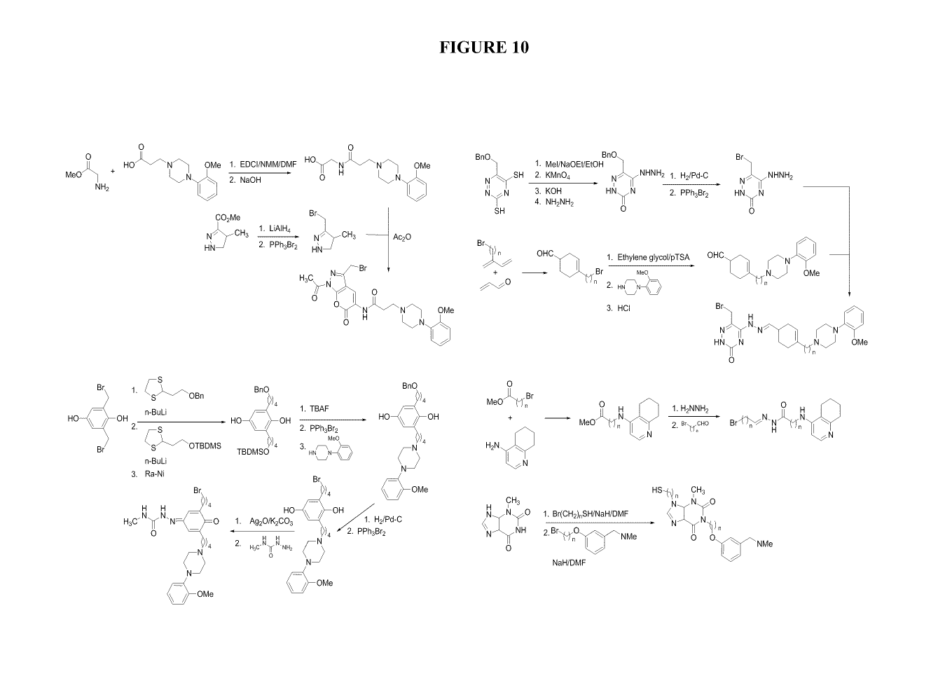

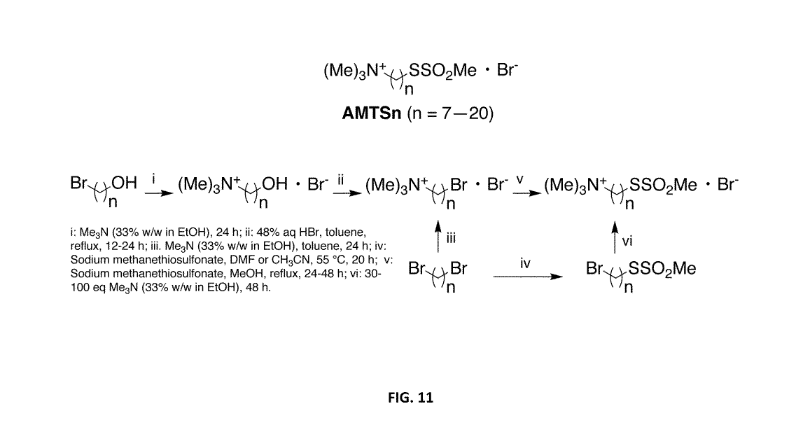

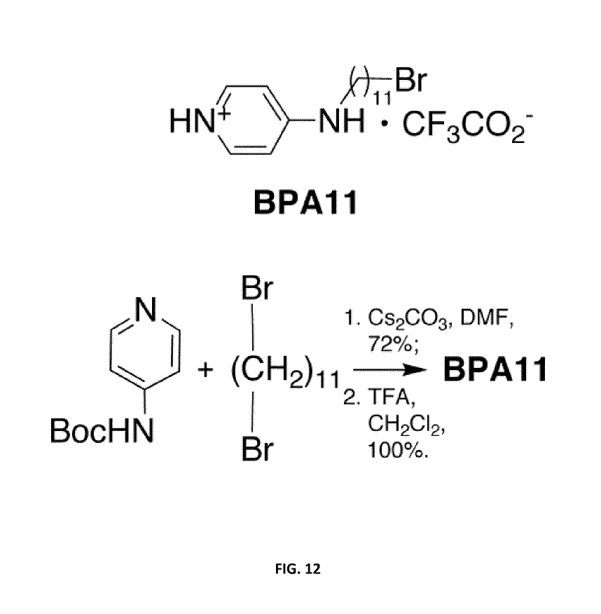

The compounds for use in the compositions and methods provided herein may be prepared by methods well known to those of skill in the art or by the methods shown herein (e.g., see FIGS. 5-12). One of skill in the art would be able to prepare all of the compounds for use herein by routine modification of these methods using the appropriate starting materials.

### D. Evaluation of the Activity of the Compounds

The activity of the compounds provided herein as inhibitors of AChE may be measured by a variety of methods for measuring AChE known to those having ordinary skill in the art; see, e.g., Pang et al., J. Biol. Chem. 271(30):23646-23649 (1996); Pang et al., PLoS ONE 4(2): e4349 (2009); Pang et al., PLoS ONE 4(8): e6851 (2009). The inhibitory activity can be evaluated in a number of species, e.g., a number of insect and mammalian species, to determine compounds that would be safe and non-toxic to mammalian species.

### E. Pesticidal and Insecticidal Compositions and Methods of Use Thereof

A pesticidal (e.g., insecticidal) composition provided herein contains one or more of the compounds provided herein and can be used to kill, or to prevent or inhibit the increase or spread of, select pest, e.g., insect, populations. A composition can optionally include an agriculturally acceptable insecticidal carrier. A composition may also contain other additive such as surfactants, emulsifiers, defoamers, buffers, thickeners, dyes, extenders, emetic agent(s) and the like. A pesticidal or insecticidal composition can be in any form, e.g., in the form of an aqueous, oil-based, or liquid spray composition, in the form of gel pellets or granules, in the form of a powder, in the form of a solid sheet, and the like.

A carrier is any material with which the active ingredient is formulated to facilitate application to the locus to be treated, which may be, without limitation, a plant, seed, grain, food, water source, or soil, or to facilitate storage, transport or handling. A carrier may be a solid or a liquid, including material which is normally a gas but which has been compressed to form a liquid or a combination thereof. Carriers include any such carriers known to those skilled in the art to be suitable for the particular mode of administration. These carriers are selected by those skilled in the art with the view to enhance handling, application to the infected sites, persistence, and storage.

An agriculturally acceptable carrier may be solid, liquid or both. Solid carries are selected from, typically, mineral earth such as silicas, silica gels, silicates, talc, kaolin, montmorillonite, attapulgite, pumice, sepiolite, bentonite, limestone, lime, chalk, bole, loes, clay, dolomite, diatomaceous earth, calcite, calcium sulfate, magnesium sulfate, magnesium sulfate, magnesium oxide, sand, ground plastics, fertilizers such as ammonium sulfate, ammonium phosphate, ammonium nitrate, ureas, and crushed products of vegetable origin such as cereal meal, tree bark meal, wood meal and nutshell meal, cellulose powders, or other solid carriers. In some embodiments, a carrier is selected from alginates, clays, activated carbons, and powdery carriers (e.g., talcum, zinc and titanium dioxide, calcined magnesia or an anhydrous metal salt).

Pesticidal or insecticidal compositions are often formulated and transported in a concentrated form which is subsequently diluted by the user before application. The presence of small amounts of a surfactant facilitates this process of dilution. Thus, a composition according to the present invention comprises, if desired, at least one surfactant. For example, the composition may contain one or more carriers and at least one surfactant.

Surfactant(s) can be non-ionic, cationic and/or anionic in nature and surfactant mixtures which have good emulsifying, dispersing and wetting properties, depending on the nature of the active ingredient to be formulated. Suitable anionic surfactants can be both water-soluble soaps and water-soluble synthetic surface-active compounds. Soaps which may be included as a surfactant can be the alkali metal, alkaline earth metal or substituted or unsubstituted ammonium salts of higher fatty acids, for example the sodium or potassium salt of oleic or stearic acid, or of natural fatty acid mixtures.

The pesticidal or insecticidal composition may be dispersed in a solid or liquid diluent for application to the pest or insect, its food supply, breeding ground or habitat as a dilute spray or as a solid dust or dust concentrate. An pesticidal or insecticidal composition is generally in a ready to use form which may be diluted at the place of application for suitable concentration of the active ingredients.

Also provided herein are methods for using the insecticidal or pesticidal compositions. For example, provided herein is a method for killing pests that includes providing a pesticidal composition described herein; and applying the pesticidal composition to an area infested with pests, such that the pests can ingest or be contacted with the pesticidal composition. The pests can be selected from mosquitoes, cockroaches, lancelets, rice leaf beetles, African bollworms, beet armyworms, codling moths, diamondback moths, domestic silkworms, honey bees, oat or wheat aphids, greenbugs, melon or cotton aphids, green peach aphids, and English grain aphids.

A method for controlling the growth or spread of a pest population is also provided, which includes treating or contacting plants, propagation stocks, seeds, grains, foodstuffs, soils, water, industrial materials, or combinations thereof with an effective amount of a pesticidal composition described herein.

Treating can include applying the composition in a manner selected from the group consisting of watering, spraying, atomizing, scattering, spreading, dry dressing, wet dressing, liquid dressing, slurry treatment of seeds, incrustation, and combinations thereof.

The insecticidal compositions can also be used to reduce or eliminate mosquito populations, e.g., mosquito populations that carry diseases. Thus, the present disclosure also provides a method of controlling the mosquito-borne spread of malaria, West Nile Virus, or encephalitis comprising applying an insecticidal composition described herein to an area infested with mosquitoes, such that the mosquitoes can ingest or be contacted with the insecticidal composition.

Finally, the pesticidal compositions can be used to control damage to crops, plants, fruits, etc. that is mediated by pests. Accordingly, a method of controlling crop, seed, bean, foodstuff, grain, or fruit damage mediated by pests can include treating or contacting the crop, seed, bean, foodstuff, grain or fruit with a pesticidal composition described herein.

### EXAMPLES

### Example 1

### Homology model of AgAChE

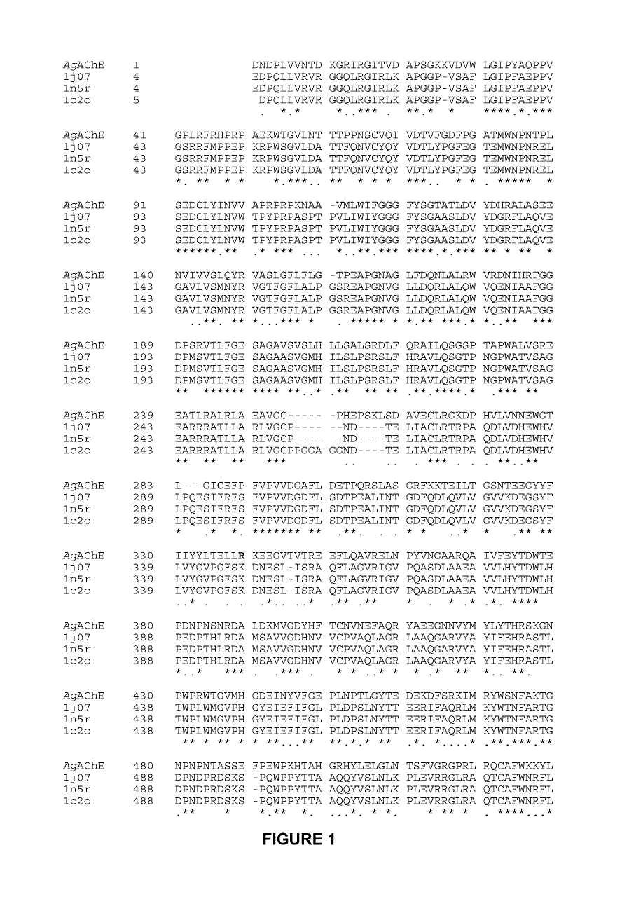

To search for a conserved and insect-specific, e.g., mosquito-specific, region of AgAChE, a 3D model of a substrate-bound AgAChE that is susceptible to current pesticides was determined. The protein sequence of this AChE was obtained from GenBank (accession number: BN000066). A homology model of AgAChE was first generated by the SWISS-MODEL program (available at swissmodel.expasy.org//SWISS-MODEL.html [7]} according to multiple sequence alignments using X-ray structures of two mouse and one electric eel AChEs as templates (FIG. 1). The Protein Data Bank (PDB) IDs of the mouse AChEs are 1J07 and 1N5R [8]; the PDB ID of the electric eel AChE is 1C2O [9]. These crystal structures were automatically identified by the SWISS-MODEL program and have the highest sequence identity (46%) to AgAChE.

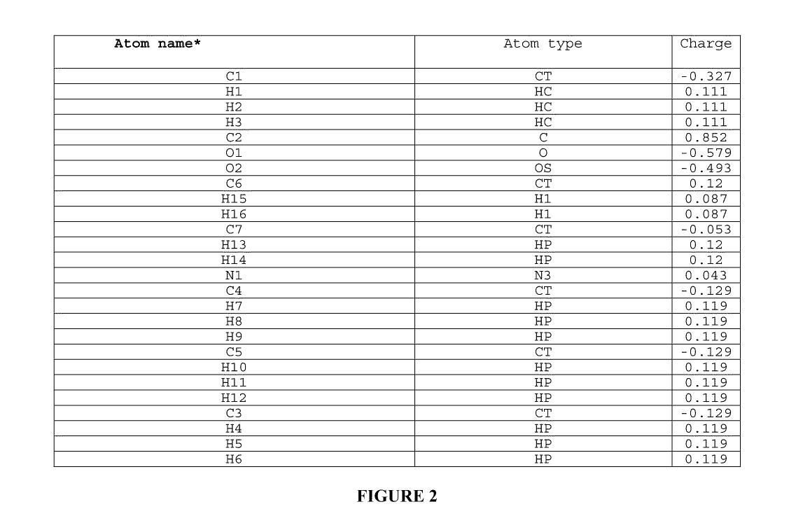

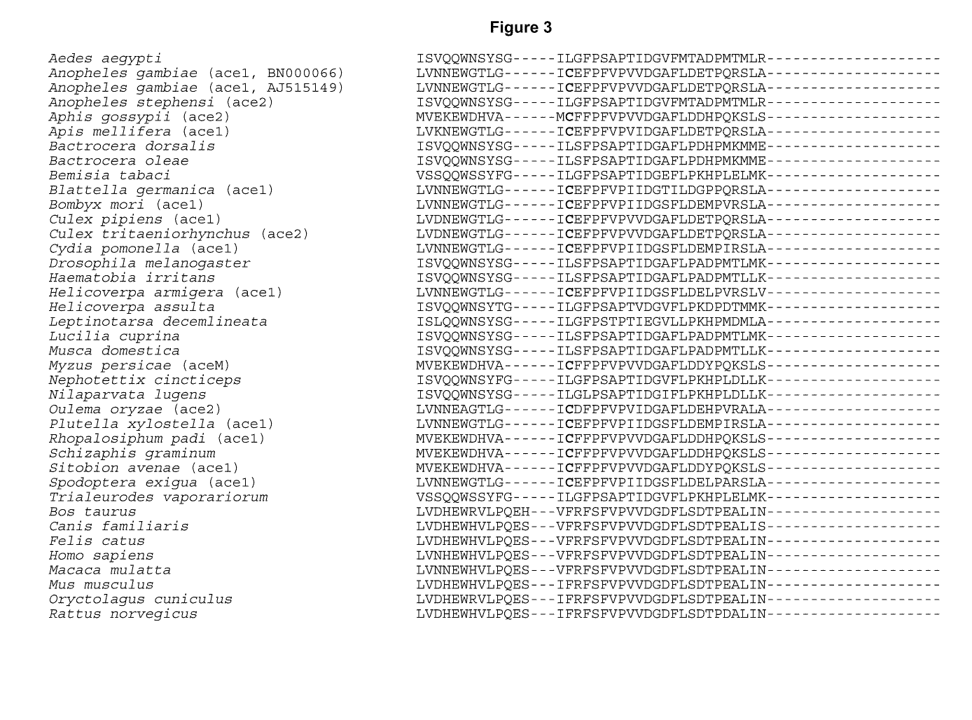

The homology model of the apo AgAChE was automatically generated by the SWISS-MODEL program. No manual adjustments were made to improve the multiple sequence alignment shown in FIG. 1. The substrate-bound AgAChE model was then built by manually docking acetylcholine into the active site of the homology model, guided by the substrate-bound Torpedo AChE (PDB ID: 2ACE [1]). The fully extended conformation of acetylcholine was used in the manual docking. The atomic charges of acetylcholine were obtained according to the RESP procedure [24] with an ab initio calculation at the HF/6-31G* level using the Gaussian98 program [25], and such charges are provided in FIG. 2.

There were four regions of insertion and four regions of deletion in the AgAChE sequence aligned with those of the crystal structures (FIG. 1). In some regions of insertion and deletion, a proline residue, known as a helix breaker, is changed to other residues in AgAChE. However, such changes do not affect the secondary structure of AgAChE, because these regions do not adopt the helical conformation in the template structures. The substrate-bound AgAChE model was then built by manually docking acetylcholine into the active site of the homology model. The docking was guided by the substrate-bound Torpedo AChE (PDB ID: 2ACE [1]).

The resulting AgAChE complex model has nearly the same backbone conformation as those of the mouse and electric eel AChE structures except for residues 280-288 (loop 2) of AgAChE, although many side-chain conformations of AgAChE are different from the corresponding ones in the mouse and electric eel enzymes. Compared to the corresponding region in the mouse and electric eel AChEs, loop 2 of AgAChE is much shorter because it contains a region of deletion (FIG. 1). Therefore, as part of the peripheral site, loop 2 of AgAChE required extensive refinement. Additionally, at the opening of the active site of the unrefined AgAChE complex model, the thiol group of C286 at loop 2 pointed away from W280 and Y333, suggesting that C286 does not interact with W280 and Y333; furthermore, the guanidino group of R339 was not accessible to solvent as it was immediately surrounded by F75, F78, Y332, and W431.

### Example 2

### Refined model of AgAChE

The homology complex model was then refined by multiple molecular dynamics simulations (MMDSs). The stochastic sampling of protein conformations achieved by MMDSs is more efficient than the sampling by a single long molecular dynamics simulation [10-15], and it is effective in refining loop conformations [15]. MMDS refinement has been previously validated through the successful identification of small-molecule inhibitors of an MMDS-refined 3D model of a protease [15,16]. The MMDS refinement method has also proven successful in refining a homology model, provided by the Protein Structure Prediction Centre (TMR01, available at http://predictioncenter.org/caspR/), to a refined model that was nearly identical to the corresponding crystal structure (Protein Data Bank ID: 1XE1). Relative to the 1XE1 crystal structure, the alpha carbon root mean square deviation of the refined model was 1.7 Å, whereas the alpha carbon root mean square deviation of the homology model was 4.6 Å. The delta alpha carbon root mean square deviation for the MMDS-refined model is −2.9 Å. This result confirmed the effectiveness of MMDSs in loop refinement, and thus MMDSs were used to refine AgAChE, especially its loop 2 region. In refining the homology model of the AgAChE complex, 100 different molecular dynamics simulations (2.0 ns for each simulation with a 1.0-fs time step and with a different seed for starting velocity) were performed according to a published protocol [15]. An average of 50,000 trajectories of the complex obtained at 1.0-ps intervals during the last 500 ps of the 100 simulations was used as a refined 3D model of AgAChE. The refined model was deposited to PDB on Sep. 10, 2005 (PDB ID: 2AZG) and released at PDB on Sep. 19, 2006.

All MMDSs were performed according to a published protocol [15] using the SANDER module of the AMBER 8.0 program [26] with the Cornell et al. force field (parm96.dat) [27]. The topology and coordinate files used in the MMDSs were generated by the PREP, LINK, EDIT, and PARM modules of the AMBER 5.0 program [26]. All simulations used (1) a dielectric constant of 1.0; (2) the Berendsen coupling algorithm [28]; (3) a periodic boundary condition at a constant temperature of 300 K and a constant pressure of 1 atm with isotropic molecule-based scaling; (4) the Particle Mesh Ewald method to calculate long-range electrostatic interactions [29]; (5) iwrap=1; (6) a time step of 1.0 fs; (7) the SHAKE-bond-length constraints applied to all the bonds involving the H atom; (8) default values of all other inputs of the SANDER module. The initial structure of the substrate-bound AgAChE used in the MMDSs had no structural water molecules, and was solvated with 16,184 TIP3P water molecules [30] (EDIT input: NCUBE=10, QH=0.4170, DISO=2.20, DISH=2.00, CUTX=8.0, CUTY=8.0, and CUTZ=8.0). The solvated AgAChE complex system had a total of 56,926 atoms; it was first energy-minimized for 200 steps to remove close van der Waals contacts in the system, slowly heated to 300 K (10 K/ps), and then equilibrated for 1.5 ns. The energy minimization used the default method of AMBER 5.0 (10 cycles of the steepest descent method followed by the conjugate gradient method). The CARNAL module was used for geometric analysis and for obtaining the time-average structure. All MMDSs were performed on 200 Apple G5 processors dedicated to the Computer-Aided Molecular Design Laboratory

Compared to the unrefined model and human AChE (hAChE), the refined model has different main-chain conformations in three adjacent loops of residues: 70-77 (loop 1), 280-288 (loop 2), and 333-349 (loop 3), that comprise most of the peripheral site of AChE. In contrast to the unrefined model, the refined model has the thiol group of C286 interacting with W280 and Y333 via sulfur-aromatic interaction [17] and the guanidino group of R339 partially accessible to solvent. The latter was caused by the side-chain conformational changes of F75 and Y332 and by the conformational change of loop 1.

### Example 3

### Identification of Invertebrate (Insect)-Specific Residues of AChE

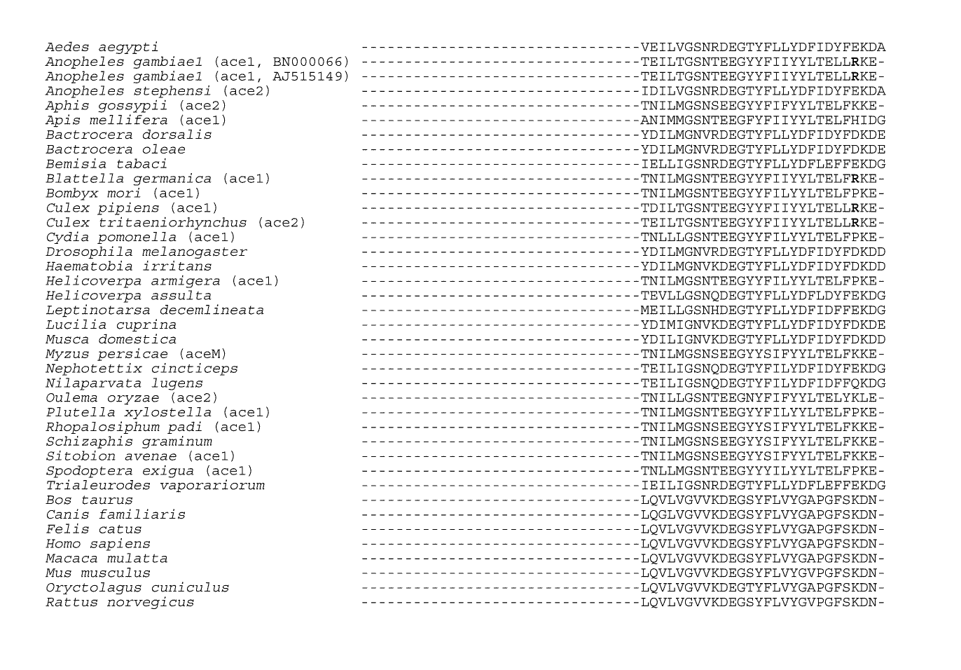

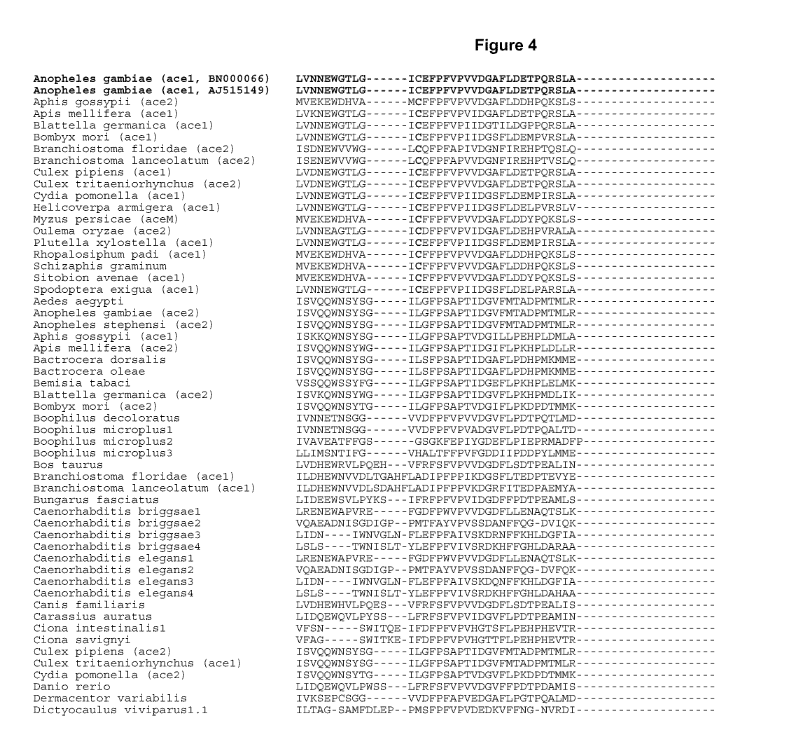

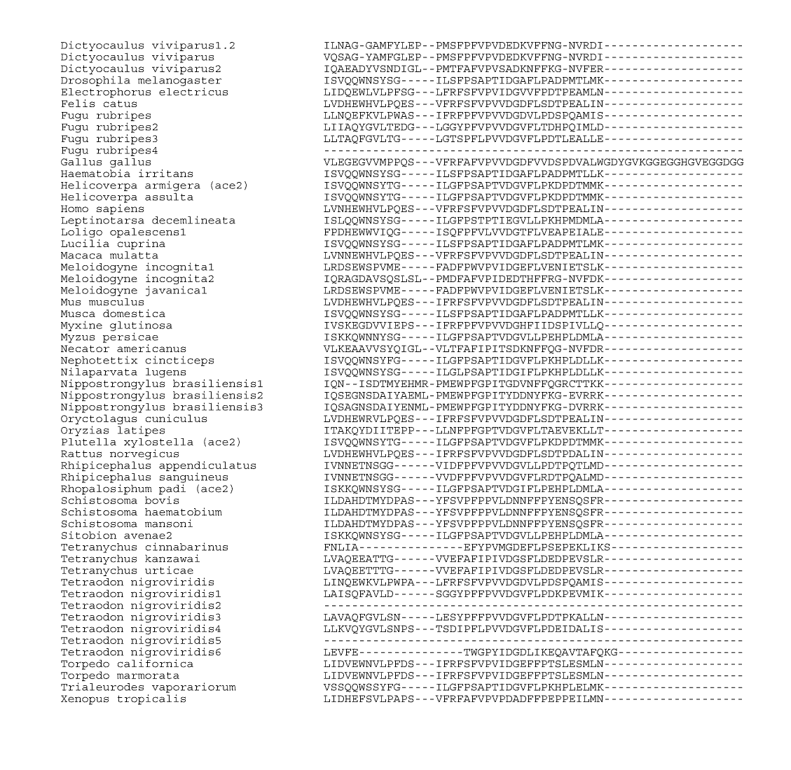

Located at the peripheral site of the refined AgAChE model, R339 has cation-pi interactions with F75, F78, Y332, and W431; this cationic residue stabilizes the aromatic residues that comprise part of the active site. The stabilizing role suggests that R339 is a conserved residue in mosquito AChEs. Interestingly, the residue corresponding to R339 of AgAChE is absent in human AChE (hAChE); instead the phenol group of Y77 of hAChE occupies the region that corresponds to the region occupied by the guanidinium group of the R339. As shown in FIG. 3, using the CLUSTALW program [18], a sequence analysis of AChEs from 73 species that are currently available shows that R339 of AgAChE is conserved in AChEs of only four insect species and absent in AChEs of all other species. Of the 73 species, 30 and 8 of them are insects and mammals, respectively. The four insect species that have the conserved Arg (R) are house mosquito (Culex pipiens), Japanese encephalitis-carrying mosquito (Culex tritaeniorhynchus), African malaria-carrying mosquito (Anopheles gambiae), including the one that is resistant to current pesticides (the G119S mutant, GenBank ID: AJ515149 [4]), and German cockroach (Blattella germanica).

Located on the opposite side of R339, C286 has favorable sulfur-aromatic interactions [17] with W280 and Y333, both located at the opening of the active site. In hAChE, the residue corresponding to C286 of AgAChE is F295 that is located in the middle of the active site. The change of C286 to F295 in loop 2 has a large displacement; the distance between two alpha carbon atoms of C286 and F295 in the overlay of the two structures is 4.8 Å. As shown in FIG. 3, a sequence analysis of AChEs from the 73 species shows that C286 is present in AChEs of 17 invertebrate species and absent in AChEs of all other species. The 17 invertebrates include house mosquito (Culex pipiens), Japanese encephalitis-carrying mosquito (Culex tritaeniorhynchus), African malaria-carrying mosquito (Anopheles gambiae) including the one that is resistant to current pesticides {GenBank ID: AJ515149 [4]}, German cockroach (Blattella germanica),

Florida lancelet (Branchiostoma floridae), rice leaf beetle (Oulema oryzae), African bollworm (Helicoverpa armigera), beet armyworm (Spodoptera exigua), codling moth (Cydia pomonella), diamondback moth (Plutella xylostella), domestic silkworm (Bombyx mori), honey bee (Apis mellifera), oat or wheat aphid (Rhopalosiphum padi), the greenbug (Schizaphis graminum), melon or cotton aphid (Aphis gossypii), green peach aphid (Myzus persicae), and English grain aphid (Sitobion avenae).

### Example 4

### Use of 3D Model to Design Inhibitors

It has been reported that a native or engineered cysteine residue near the active site of an enzyme can bind a small molecule that interacts, even loosely, at the active site, as long as the cysteine residue is able to react with an electrophilic group of the molecule [19]. It has also been reported that reactive chemicals which are covalently bonded to an engineered cysteine (H287C) at the peripheral site of mammalian AChEs are able to interfere with substrate binding and subsequently inhibit the enzymes [20, 21]. Furthermore, it has been reported that upon binding in proximity of a native cysteine residue at the active site of a cysteine protease, a chemically stable molecule is able to bond covalently to the cysteine residue [22]. Based on these reports and on the proximity of C286 to its active site revealed by the 3D model of AgAChE described herein, it is conceivable that a chemically stable molecule can react with C286 and irreversibly inhibit AgAChE upon binding to the active site.

Because of their species specificity demonstrated by the sequence analysis, C286 and R339 can be used as species markers for developing effective and safer pesticides that can covalently bond to C286 and noncovalently to R336 of AgAChE. The absence of a cysteine residue in the peripheral site of mammalian AChEs means that pesticides targeting C286 and R339 would have less toxicity to mammals than current pesticides targeting the catalytic serine residue present in both mammals and insects.

The aforementioned sequence analysis shows that both R339 and C286 are conserved in AChEs of African malaria-carrying mosquito (Anopheles gambiae), Japanese encephalitis-carrying mosquito (Culex tritaeniorhynchus), and house mosquito (Culex pipiens). The two residues are conserved also in the African malaria-carrying mosquito AChE mutant that is resistant to current pesticides [4]. The above-described structural analysis demonstrates that R339 interacts with F75, F78, Y332, and W431, and that C286 interacts with W280 and Y333. All of these aromatic residues contribute importantly to the aromaticity of the active site of AChE that is required to bind its cationic substrate; R339 and C296 play a role in stabilizing these aromatic residues and conceivably have low mutation rates. Therefore, pesticides targeting R339 and C296 of AgAChE and the other insect species might be devoid of the mammalian toxicity and the resistance problems of current pesticides.

Accordingly, virtual screening against the 3D model of AgAChE using a published protocol [16,23] was used to identify small molecules that have functional groups capable of interacting with R339, e.g., with an interaction energy in the range of from about −5 to about −60 kcal/mol, or in some cases from about −20 to −40 kcal/mol. These molecules were used to design molecules that were expected to interact simultaneously with C286 and R339, given that the average distance of the sulfur atom of C286 to the guanidine carbon atom of R339 is 13 Å, and also with W84 (Trp84) present in the active site. See Formulas I-VIII, above. Thus, because the guanidinium group of an arginine residue has multiple hydrogen bond donors and interacts favorably with aromatic groups, R339 can be used as an anchor to facilitate the reaction of an inhibitor with C286 of AgAChE. The unique presence of R339 and C286 in AgAChE and the corresponding R and C groups in certain of the other insect species permits the design of molecules capable of acting as suicide inhibitors that first interacts with R339, leaving an electrophile in the proximity of C286 that can react with C286; see also [22].

### Example 5

### Reaction Schemes

The compositions described above can be prepared using the synthetic schemes set forth in FIGS. 5-12.

### REFERENCES

1. Raves M L, Harel M, Pang Y-P, Silman I, Kozikowski A P, et al. (1997) Structure of acetylcholinesterase complexed with the nootropic alkaloid, (−)-huperzine A. Nature Structural Biology 4: 57-63.
2. Pang Y—P, Quiram P, Jelacic T, Hong F, Brimijoin S (1996) Highly potent, selective, and low cost bis-tetrahydroaminacrine inhibitors of acetylcholinesterase. Steps toward novel drugs for treating Alzheimer's disease. Journal of Biological Chemistry 271: 23646-23649.
3. Fialka J J (May 25, 2006) EPA scientists cite pressure in pesticide study. Wall Street Journal. Washington. pp. A4.
4. Weill M, Lutfalla G, Mogensen K, Chandre F, Berthomieu A, et al. (2003) Comparative genomics: Insecticide resistance in mosquito vectors. Nature 423: 136-137.
5. Filler S J, MacArthur J R, Parise M, Wirtz R, Eliades M J, et al. (2006) Locally acquired mosquito-transmitted malaria: A guide for investigations in the United States. MMWR Morb Mortal Wkly Rep 55: 1-9.
6. Weill M, Malcolm C, Chandre F, Mogensen K, Berthomieu A, et al. (2004) The unique mutation in ace-1 giving high insecticide resistance is easily detectable in mosquito vectors. Insect Molecular Biology 13: 1-7.
7. Schwede T, Kopp J, Guex N, Peitsch M C (2003) SWISS-MODEL: An automated protein homology-modeling server. Nucleic Acids Res 31: 3381-3385.
8. Bourne Y, Taylor P, Radic Z, Marchot P (2003) Structural insights into ligand interactions at the acetylcholinesterase peripheral anionic site. Embo J 22: 1-12.
9. Bourne Y, Grassi J, Bougis P E, Marchot P (1999) Conformational flexibility of the acetylcholinesterase tetramer suggested by x-ray crystallography. J Biol Chem 274: 30370-30376.
10. Caves L S D, Evanseck J D, Karplus M (1998) Locally accessible conformations of proteins—multiple molecular dynamics simulations of crambin. Protein Science 7: 649-666.
11. Smith L J, Daura X, van Gunsteren W F (2002) Assessing equilibration and convergence in biomolecular simulations. Proteins: Structure, Function & Genetics 48: 487-496.
12. Snow C D, Nguyen N, Pande V S, Gruebele M (2002) Absolute comparison of simulated and experimental protein-folding dynamics. Nature 420: 102-106.
13. Zagrovic B, Snow C D, Shirts M R, Pande V S (2002) Simulation of folding of a small alpha-helical protein in atomistic detail using worldwide-distributed computing. Journal of Molecular Biology 323: 927-937.
14. Oelschlaeger P, Schmid R D, Pleiss J (2003) Modeling domino effects in enzymes: Molecular basis of the substrate specificity of the bacterial metallo-beta-lactamases IMP-1 and IMP-6. Biochemistry 42: 8945-8956.
15. Pang Y-P (2004) Three-dimensional model of a substrate-bound SARS chymotrypsin-like cysteine proteinase predicted by multiple molecular dynamics simulations: catalytic efficiency regulated by substrate binding. Proteins 57: 747-757.
16. Dooley A J, Shindo N, Taggart B, Park J G, Pang Y-P (2006) From genome to drug lead: identification of a small-molecule inhibitor of the SARS virus. Bioorg Med Chem Lett 16: 830-833.
17. Zauhar R J, Colbert C L, Morgan R S, Welsh W J (2000) Evidence for a strong sulfur-aromatic interaction derived from crystallographic data. Biopolymers 53: 233-248.
18. Chema R, Sugawara H, Koike T, Lopez R, Gibson T J, et al. (2003) Multiple sequence alignment with the Clustal series of programs. Nucleic Acids Research 31: 3497-3500.
19. Erlanson D A, Braisted A C, Raphael D R, Randal M, Stroud R M, et al. (2000) Site-directed ligand discovery. Proceedings of the National Academy of Sciences of the United States of America 97: 9367-9372.
20. Boyd A E, Marnett A B, Wong L, Taylor P (2000) Probing the active center gorge of acetylcholinesterase by fluorophores linked to substituted cysteines. J Biol Chem 275: 22401-22408.
21. Johnson J L, Cusack B, Hughes T F, McCullough E H, Fauq A, et al. (2003) Inhibitors tethered near the acetylcholinesterase active site serve as molecular rulers of the peripheral and acylation sites. J Biol Chem 278: 38948-38955.
22. Pang Y-P, Xu K, Kollmeyer T M, Perola E, McGrath W J, et al. (2001) Discovery of a new inhibitor lead of adenovirus proteinase: steps toward selective, irreversible inhibitors of cysteine proteinases. FEBS Letters 502: 93-97.
23. Perola E, Xu K, Kollmeyer T M, Kaufmann S H, Prendergast F G, et al. (2000) Successful virtual screening of a chemical database for farnesyltransferase inhibitor leads. J Med Chem 43: 401-408.
24. Cieplak P, Cornell W D, Bayly C, Kollman P A (1995) Application of the multimolecule and multiconformational resp methodology to biopolymers: charge derivation for DNA, RNA, and proteins. Journal of Computational Chemistry 16: 1357-1377.
25. Frisch M J, Trucks G W, Schlegel H B, Gill P M W, Hohnson B G, et al. (1999) GAUSSIAN 98, Revision A.7. Gaussian, Inc Pittsburgh, Pa.
26. Pearlman D A, Case D A, Caldwell J W, Ross W S, Cheatham III T E, et al. (1995) AMBER, a package of computer programs for applying molecular mechanics, normal mode analysis, molecular dynamics and free energy calculations to simulate the structural and energetic properties of molecules. Comput Phys Commun 91: 1-41.
27. Cornell W D, Cieplak P, Bayly C, Gould I R, Merz Jr. K M, et al. (1995) A second generation force field for the simulation of proteins, nucleic acids, and organic molecules. Am Chem Soc 117: 5179-5197.
28. Berendsen H J C, Postma J P M, van Gunsteren W F, Di Nola A, Haak J R (1984) Molecular dynamics with coupling to an external bath. J Chem Phys 81: 3684-3690.
29. Darden T A, York D M, Pedersen L G (1993) Particle Mesh Ewald: An N log(N) method for Ewald sums in large systems. J Chem Phys 98: 10089-10092.
30. Jorgensen W L, Chandreskhar J, Madura J D, Impey R W, Klein M L (1982) Comparison of simple potential functions for simulating liquid water. J Chem Phys 79: 926-935.
31. Pang Y P, Singh S K, Gao Y, Lassiter T L, Mishra R K, Zhu K Y, and Brimijoin S (2009) Selective and Irreversible Inhibitors of Aphid Acetylcholinesterases: Steps Toward Human-Safe Insectivides. PLoS ONE 4(2): e4349.
32. Pang Y P, Ekstrom F, Polsinelli G, Gao Y, Rana S, Hua D H, Andersson B, Andersson P O, Peng L, Sing S K, Mishra R K, Zhu K Y, Fallon A M, Ragsdale D W, Brimijoin S (2009) Selective and Irreversible Inhibitors of Mosquito Acetylcholinesterases for Controlling Malaria and Other Mosquito-Borne Diseases. PLoS ONE 4(8): e6851.

A number of embodiments of the invention have been described. Nevertheless, it will be understood that various modifications may be made without departing from the spirit and scope of the invention. Accordingly, other embodiments are within the scope of the following claims.

What is claimed is:

1. A composition of matter comprising a compound according to Formula I or an acceptable salt or derivative thereof: 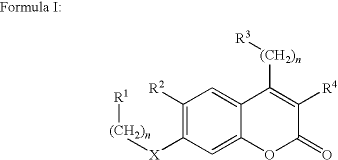
wherein n=0 to 3;
wherein R¹ is selected from:
a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 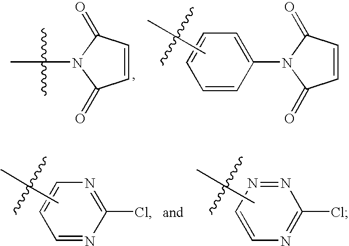
and X₅=alkyl(C₁-C₅) or —(O)₂alkyl(C₁-C₅); and
b) H and one of the moieties A, B, C, D, and E: 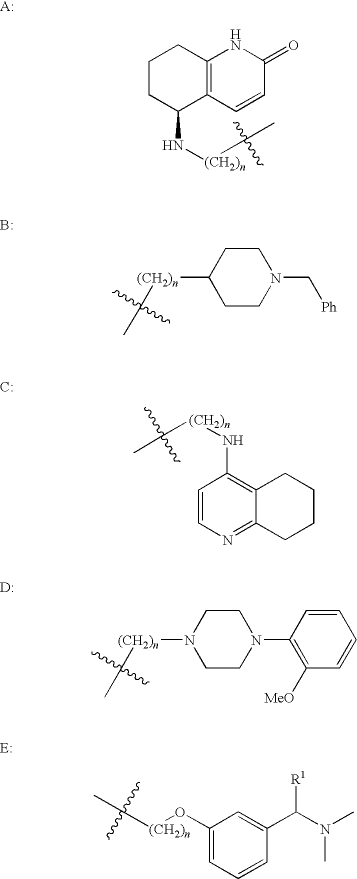
wherein E's R¹═H or Me; and n for A-E ranges from 0 to 5;
wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10, X₄ is as above, and X₅=alkyl(C₁-C₅) or —(O)₂alkyl(C₁-C₅);
wherein R³ is selected from H, CH₃, and
a) —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅, where n=1 to 10, X⁴ is as above, and X₅=alkyl(C₁-C₅) or —(O)₂alkyl(C₁-C₅); and
b) one of the moieties A, B, C, D, and E: 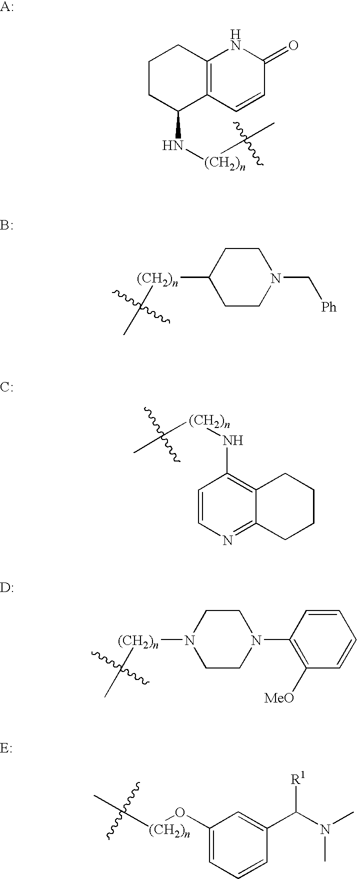
wherein E's R¹═H or Me; and n for A-E ranges from 0 to 5;
wherein R⁴ is selected from:
H and one of the moieties A, B, C, D, and E: 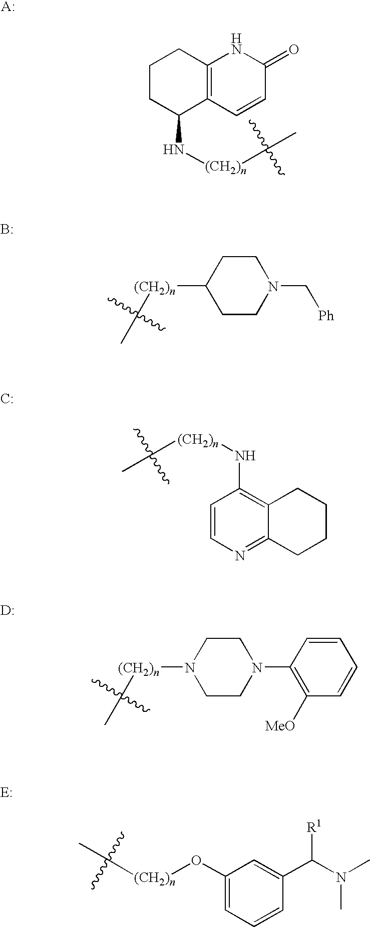
wherein E's R¹═H or Me; and n for A-E ranges from 0 to 5;
and
wherein X═O or NR⁵, wherein R⁵ is selected from H or C₁-C₅ alkyl.

2. A composition of matter comprising a compound according to Formula II or an acceptable salt or derivative thereof: 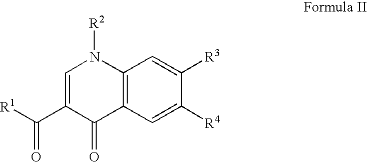
wherein R¹ is H, OMe, OEt, NH₂, alkyl(C₁-C₅), or is selected from:
A, B, C, D, and E: 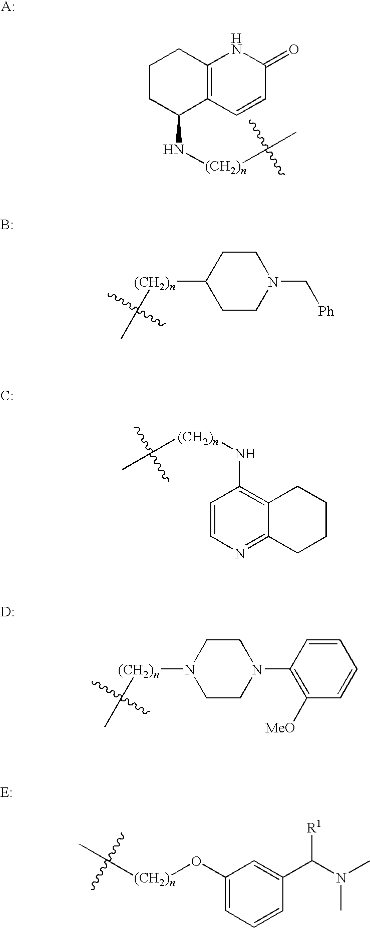
wherein E's R¹═H or Me; and n for A-E ranges from 0 to 5;
wherein R² and R³ are independently selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CHASH, —(CH₂)ₙS—SX₅ and H, where n=1 to 10; X₄ is selected from: 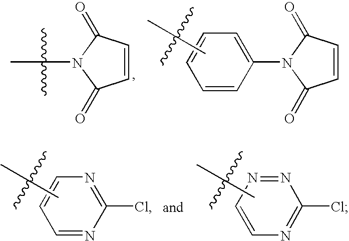
and X₅=alkyl(C₁-C₅) or —(O)₂alkyl(C₁-C₅);
wherein R⁴ is selected from: H and one of the moieties A, B, C, D, and E: 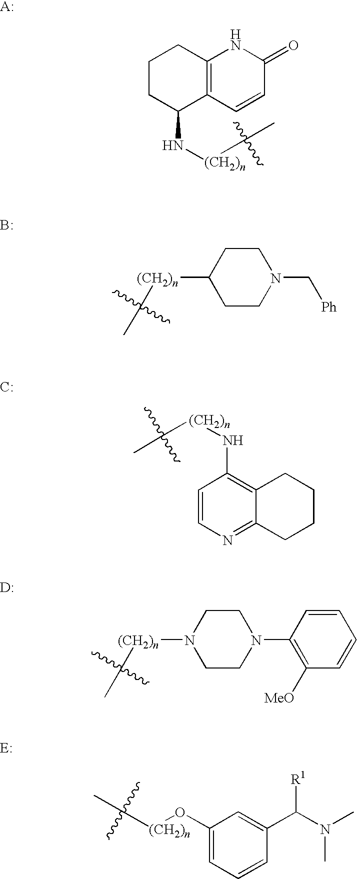
wherein E's R¹═H or Me; and n for A-E ranges from 0 to 5.

3. A composition of matter comprising a compound according to Formula IIIA or Formula IIIB or an acceptable salt or derivative thereof: 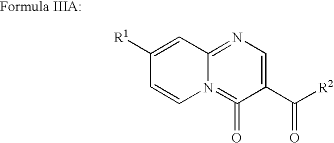
wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 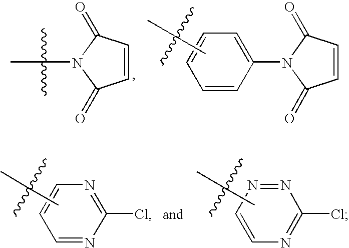
and X₅=alkyl(C₁-C₅) or —(O)₂alkyl(C₁-C₅); and
wherein R² is selected from: H and one of the moieties A, B, C, D, and E: 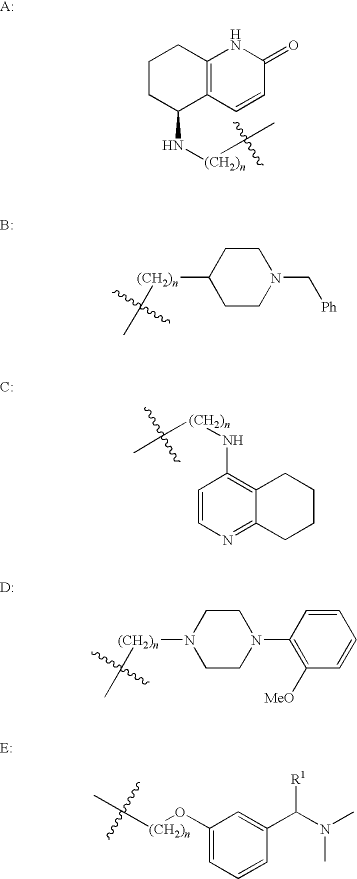
wherein E's R¹═H or Me; and n for A-E ranges from 0 to 5; or 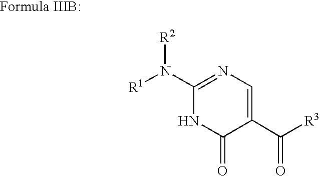
wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CHASH, —(CH₂)ₙS—SX₅ and H, where n=1 to 10; X₄ is selected from: 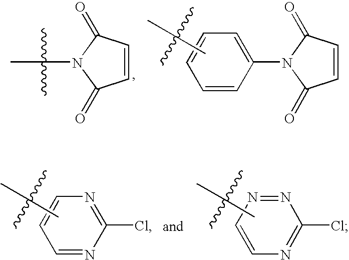
and X₅=alkyl(C₁-C₅) or —(O)₂alkyl(C₁-C₅); and
R² is selected from H and alkyl(C₁-C₅); and
R³ is selected from: H and one of the moieties A, B, C, D, and E: 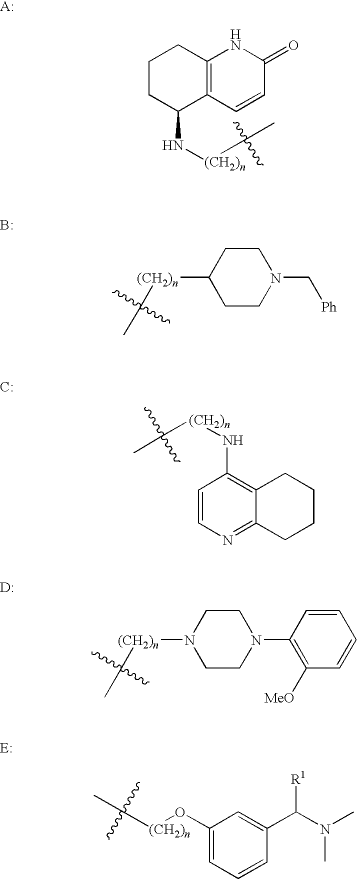
wherein E's R¹═H or Me; and n for A-E ranges from 0 to 5.

4. A composition of matter comprising a compound according to Formula IV or an acceptable salt or derivative thereof: 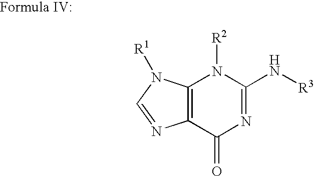
wherein R¹ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 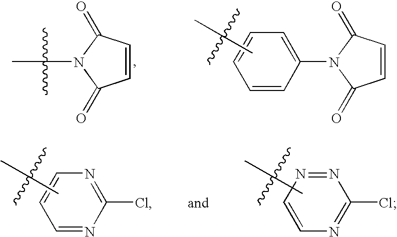
and X₅=alkyl(C₁-C₅) or —(O)₂alkyl(C₁-C₅); and
R² is selected from H and alkyl(C₁-C₅); and
R³ is selected from: H and one of the moieties A, B, C, D, and E: 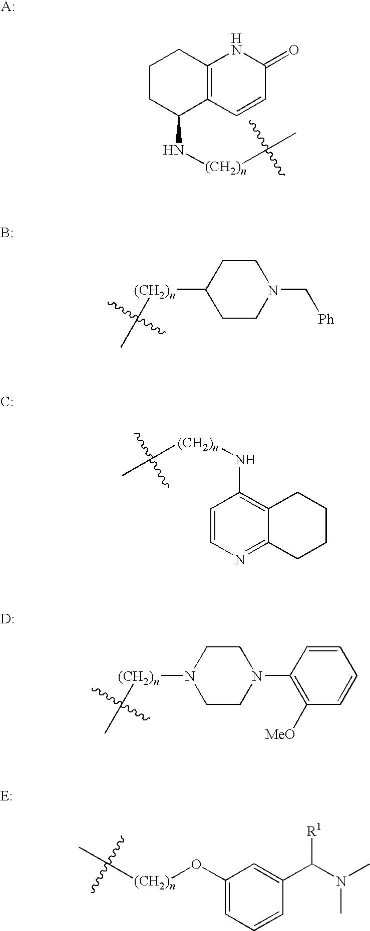
wherein E's R¹═H or Me; and n for A-E ranges from 0 to 5.

5. A composition of matter comprising a compound according to Formula V or an acceptable salt or derivative thereof: 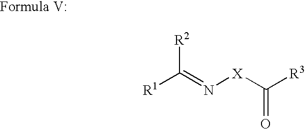
wherein X is O or NH;
wherein R¹ and R² are independently selected from H and substituted or unsubstituted, saturated or unsaturated, cyclic or linear alkyl or heteroalkyl moieties and substituted or unsubstituted aryl or heteroaryl moieties, or together R¹ and R² form a saturated or unsaturated cyclic alkyl or heteroalkyl moiety, or an aryl or heteroaryl moiety, any of which may be substituted or unsubstituted (e.g., with a fused aryl ring (which may be substituted with R⁴ and/or R⁵ moieties), or with alkyl moieties (which may be substituted with R⁴ and R⁵ moieties));
wherein R³ is selected from H, a substituted or unsubstituted alkyl, aryl, or amine moiety (e.g., substituted with R⁶ and/or R⁷ or is R⁸) or is selected from one of the moieties A, B, C, D, and E: 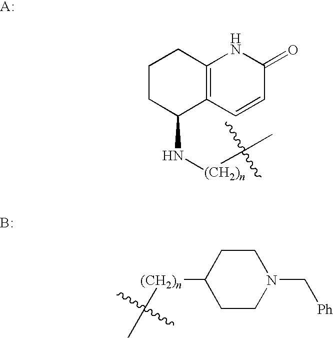 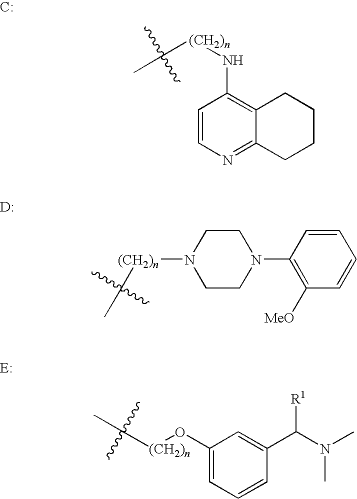
wherein E's R¹═H or Me; and n for A-E ranges from 0 to 5;
wherein R⁴ is selected from H and one of the moieties A, B, C, D, and E set forth above for R³;
wherein R⁵ is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 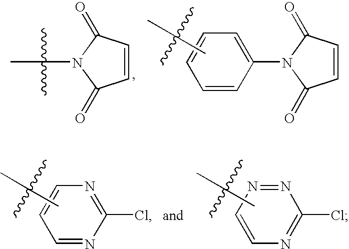
and X₅=alkyl(C₁-C₅) or —(O)₂alkyl(C₁-C₅);
wherein R⁶ and R⁷ are independently selected from H or alkyl(C₁-C₅); and
wherein R⁸ is selected from CH₃ and CH₂CH₃.

6. A composition of matter comprising a compound according to Formula VI or an acceptable salt or derivative thereof: 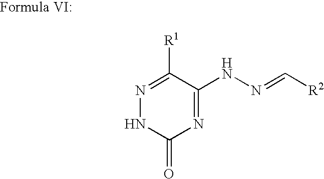
wherein R² is selected from: 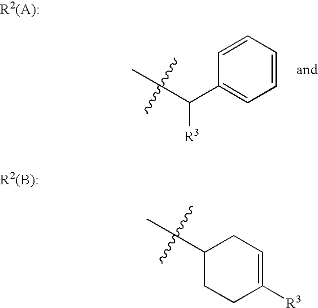
wherein R¹ is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂O, —(CH₂)ₙX₄, —(CHASH, —(CH₂)ₙS—SX₅ and H, where n=1 to 10; X₄ is selected from: 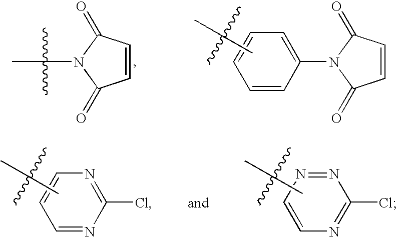
and X₅=alkyl(C₁-C₅) or —(O)₂alkyl(C₁-C₅); and
wherein R³ is selected from H and is selected from one of the moieties A, B, C, D, and E. 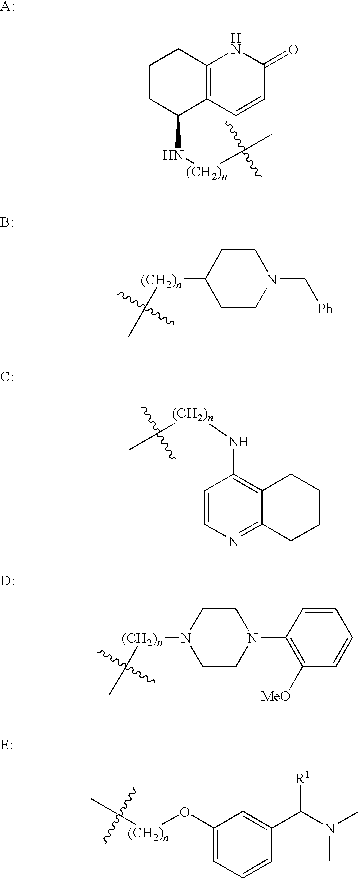
wherein E's R¹═H or Me; and n for A-E ranges from 0 to 5.

7. A composition of matter comprising a compound according to Formula VII or an acceptable salt or derivative thereof: 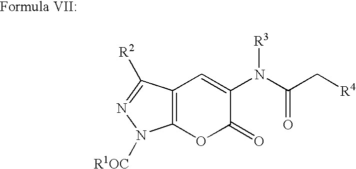
wherein R¹ and R³ are independently selected from H or alkyl(C₁-C₅);
wherein R² is selected from: —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 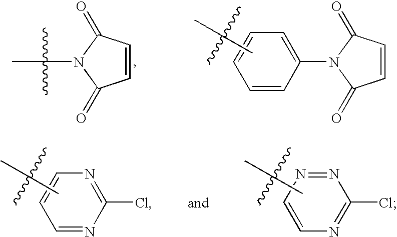
and X₅=alkyl(C₁-C₅) or —(O)₂alkyl(C₁-C₅); and
wherein R⁴ is selected from H and from one of the moieties A, B, C, D, and E: 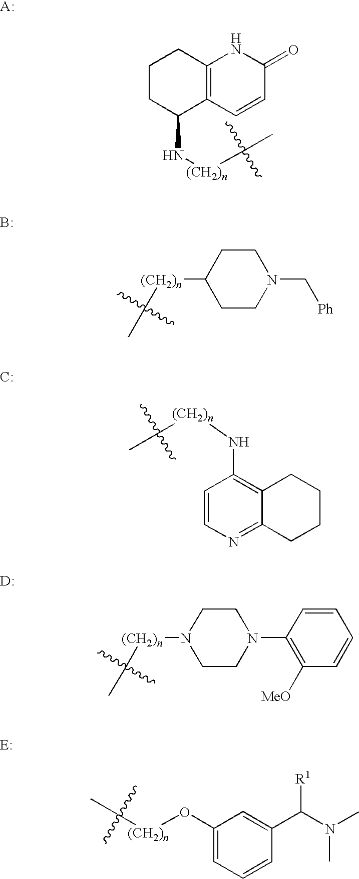
wherein E's R¹═H or Me; and n for A-E ranges from 0 to 5.

8. A composition of matter comprising a compound according to Formula VIII or an acceptable salt or derivative thereof: 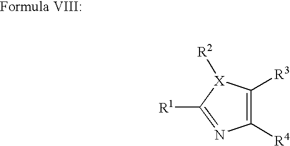
wherein R¹ is selected from H, halogen, and one of the moieties A, B, C, D, and E: 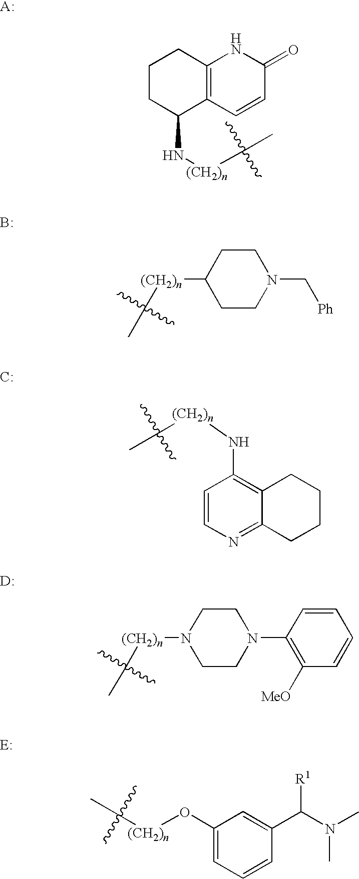
wherein E's R¹═H or Me; and n for A-E ranges from 0 to 5;
R² is selected from —(CH₂)ₙI, —(CH₂)ₙBr, —(CH₂)ₙX₄, —(CH₂)ₙSH, —(CH₂)ₙS—SX₅, (CH₂)ₙCOCH₂I, —(CH₂)ₙCOCH₂Br, —(CH₂)ₙCOCH₂Cl, —(CH₂)ₙNHCOCH₂I, —(CH₂)ₙNHCOCH₂Br, —(CH₂)ₙNHCOCH₂Cl, —(CH₂)ₙCH═CHCH₂I, —(CH₂)ₙCH═CHCH₂Br, —(CH₂)ₙCH═CHCH₂Cl, —(CH₂)ₙPhCH₂I, —(CH₂)ₙPhCH₂Br, —(CH₂)ₙPhCH₂Cl, —(CH₂)ₙX₄, —(CH₂)ₙSH, —(CH₂)ₙS—SX₅ and H, where n=1 to 10; X₄ is selected from: 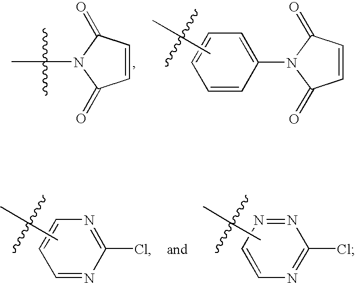
and X₅=alkyl(C₁-C₅) or —(O)₂alkyl(C₁-C₅);
wherein R³, R⁴ together form a saturated or unsaturated cyclic alkyl or cyclic heteroalkyl moiety, which may be substituted (e.g., with one or more R², R⁵, or R⁶ groups), or together form an aryl or heteroaryl moiety which may be substituted (e.g., with one or more R², R⁵, or R⁶ groups)
wherein R⁵ is selected from H or alkyl(C₁-C₅);
wherein R⁶ is selected from H and one of the moieties A, B, C, D, and E: 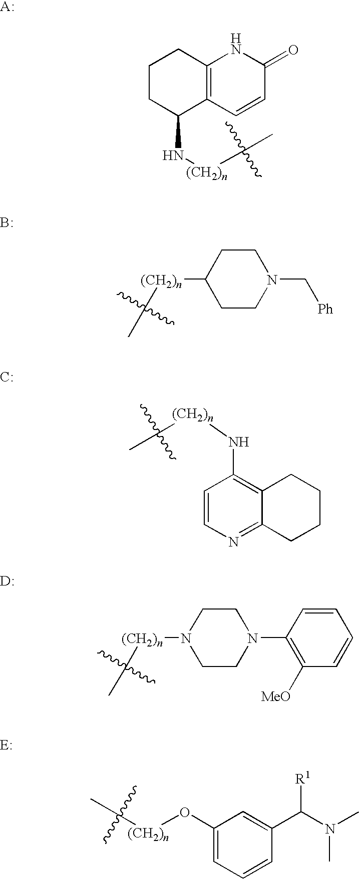
wherein E's R¹═H or Me; and n for A-E ranges from 0 to 5; and
wherein X is selected from O, S, and NH.

9. A computer-assisted method of generating a test inhibitor of the acetylcholinesterase site activity of an invertebrate acetylcholinesterase (AChE) polypeptide, the method using a programmed computer comprising a processor and an input device, the method comprising:
(a) inputting on the input device data comprising a structure of an acetylcholinesterase active site of an invertebrate AChE polypeptide;
(b) docking into the active site a test inhibitor molecule using the processor; and
(c) determining, based on the docking, whether the test inhibitor molecule would be capable of interacting with a residue of the invertebrate AChE polypeptide corresponding to Arg339 of AgAChE.

10. The method of claim 9, wherein the acetylcholinesterase active site has acetylcholine bound.

11. The method of claim 9, further comprising derivatizing the test inhibitor molecule so that the test inhibitor molecule is capable of interacting with a residue of the invertebrate AChE polypeptide corresponding to Cys286 of AgAChE.

12. The method of claim 9 or 11, further comprising derivatizing the test inhibitor molecule so that the test inhibitor molecule is capable of interacting with a residue of the invertebrate AChE polypeptide corresponding to Trp84 of AgAChE.

13. The method of claim 11, further comprising evaluating the inhibitory activity of the test inhibitor on an invertebrate or mammalian AChE polypeptide in vitro.

14. The method of claim 9, wherein said invertebrate AChE polypeptide is Anopheles gambiae AChE, Culex pipiens AChE, or Culex tritaeniorhynchus AChE.

15. The method of claim 13, further comprising evaluating the inhibitory activity of the test inhibitor on the growth of a eukaryotic cell.

16. The method of claim 15, wherein said eukaryotic cell is a mammalian cell.

17. A method of generating a compound that inhibits the acetylcholinesterase site activity of the Anopheles gambiae AChE polypeptide, the method comprising:
(a) providing a three-dimensional structure of the Anopheles gambiae AChE polypeptide acetylcholinesterase active site; and
(b) designing, based on the three-dimensional structure, a test compound capable of interacting with Arg339.

18. The method of claim 17, wherein said test compound is further capable of interacting with Cys286.

19. The method of claim 18, wherein said test compound is further capable of interacting with W84.

20. An insecticidal composition comprising a compound, or salt or derivative thereof, according to any one of claims 1-8, 29 or 30 and a carrier.

21. A pesticidal composition comprising a compound, or salt or derivative thereof, according to any one of claims 1-8, 29 or 30 and a carrier.

22. A method for killing pests comprising: providing a pesticidal composition according to claim 21; and applying the pesticidal composition to an area infested with pests, such that the pests can ingest or be contacted with the pesticidal composition.

23. The method according to claim 22, wherein the pests are selected from mosquitoes, cockroaches, lancelets, rice leaf beetles, African bollworms, beet armyworms, codling moths, diamondback moths, domestic silkworms, honey bees, oat or wheat aphids, greenbugs, melon or cotton aphids, green peach aphids, and English grain aphids.

24. A method to eliminate pests which comprises applying to a surface to be treated a pesticidal composition according to claim 21.

25. A method for controlling the growth or spread of a pest population, comprising treating or contacting plants, propagation stocks, seeds, grains, foodstuffs, soils, water, industrial materials, or combinations thereof with an effective amount of a pesticidal composition according to claim 21.

26. The method according to claim 25, wherein treating comprises applying the composition in a manner selected from the group consisting of watering, spraying, atomizing, scattering, spreading, dry dressing, wet dressing, liquid dressing, slurry treatment of seeds, incrustation, and combinations thereof.

27. A method of controlling the mosquito-borne spread of malaria, West Nile Virus, or encephalitis comprising applying an insecticidal composition according to claim 20 to an area infested with mosquitoes, such that the mosquitoes can ingest or be contacted with the insecticidal composition.

28. A method of controlling crop, seed, bean, foodstuff, grain, or fruit damage mediated by pests comprising treating or contacting the crop, seed, bean, foodstuff, grain or fruit with a pesticidal composition according to claim 21.

29. A composition of matter comprising a compound according to Formula IX or an acceptable salt or derivative thereof: 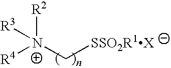
wherein R¹, R², R³ and R⁴ are independently selected from is selected from H or alkyl(C₁-C₅);
X is selected from halo and pseudohalo; and
n is an integer from 5 to 25.

30. A composition of matter comprising a compound according to Formula X or an acceptable salt or derivative thereof: 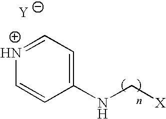
wherein X is selected from halo and pseudohalo;
Y is an salt; and
n is an integer from 5 to 15.

30. A computer-assisted method of generating a test inhibitor of the acethylcholinesterase site activity of an invertebrate acetylcholinesterase (AChE) polypeptide, the method comprising:
(a) receiving on a computing device data comprising a structure of an acetylcholinesterase active site of an invertebrate AChE polypeptide;
(b) docking into the docking box a test inhibitor molecule using the computing device; and
(c) determining, using the computing device, based on the docking, whether the test inhibitor molecule would be capable of interacting with a residue of the invertebrate AChE polypeptide corresponding to Arg339 of AgAChE.

31. A computer-assisted method of generating a test inhibitor of the acethylcholinesterase site activity of an invertebrate acetylcholinesterase (AChE) polypeptide, the method comprising:
(a) receiving on a computing device data comprising a structure of an acetylcholinesterase active site of an invertebrate AChE polypeptide;
(b) docking into the docking box a test inhibitor molecule using the computing device; and
(c) determining, using the computing device, based on the docking, whether the test inhibitor molecule would be capable of interacting with a residue of the invertebrate AChE polypeptide corresponding to Cys289 of greenbug AChE.

32. A computer-assisted method of generating a test inhibitor of the acetylcholinesterase site activity of an invertebrate acetylcholinesterase (AChE) polypeptide, the method using a programmed computer comprising a processor and an input device, the method comprising:
(a) inputting on the input device data comprising a structure of an acetylcholinesterase active site of an invertebrate AChE polypeptide;
(b) docking into the active site a test inhibitor molecule using the processor; and
(c) determining, based on the docking, whether the test inhibitor molecule would be capable of interacting with a residue of the invertebrate AChE polypeptide corresponding to Cys289 of greenbug AChE.

33. The method of claim 31, wherein the acetylcholinesterase active site has acetylcholine bound.

34. The method of claim 31, further comprising derivatizing the test inhibitor molecule so that the test inhibitor molecule is capable of interacting with a residue of the invertebrate AChE polypeptide corresponding to Trp87 of greenbug AChE.

35. The method of claim 34, further comprising evaluating the inhibitory activity of the test inhibitor on an invertebrate or mammalian AChE polypeptide in vitro.

36. The method of claim 31, wherein said invertebrate AChE polypeptide is Anopheles gambiae AChE, Culex pipiens AChE, or Culex tritaeniorhynchus AChE.

37. The method of claim 36, further comprising evaluating the inhibitory activity of the test inhibitor on the growth of a eukaryotic cell.

38. The method of claim 37, wherein said eukaryotic cell is a mammalian cell.

39. A method of generating a compound that inhibits the acetylcholinesterase site activity of the greenbug AChE polypeptide, the method comprising:
(a) providing a three-dimensional structure of the greenbug AChE polypeptide acetylcholinesterase active site; and
(b) designing, based on the three-dimensional structure, a test compound capable of interacting with Cys289.

40. The method of claim 39, wherein said test compound is further capable of interacting with Trp87.
